SEQUENCE LISTING (147 sequences)

SEQ ID NO: 1 - DNA, 1376 bp, Cellulomonas uda
  source 1..1376: mol_type = genomic DNA
atgtccgcccgcagaaccgcgtccgcgctgctgaccgccgcagcgttgtccgtcacaggcctcacggccgtcgcgagcaccgcgagcgcagccgacggctgccgcgtcgactacaccgtccagagccagtggaactccgggttccagggcgacgtcaagatcaccaacctggggtccccgctctcgtcgtggaccgtcgcgtggacgttcccgtcctcgggccagcgcgtctcgcagtcgtggaacgcgaacgtcacgacgagcggctcgcgcgtctccgcgtcgagcatcggctggaacggctcgctcggcacgggtgcgtcgacccagttcggcttcatcggctcgttcaccgactcgaacccggtgccgacgtcgttcacggtcaacggcgtgacgtgcacgggcaccgtcaccccgacgcagggccccacgcagggtccgacccagggccccacgcagggaccgacccagggccccacgcagggtccgacgcagggaccgacccagggccccacgcagaacccgcagccgtcgggtgacctgtacgtcgacaccgagaaccagtcgtacgccgcctggcaggctgcctccggcagcaccaaggacctcctggccaagatcgcgctgaccccccagtcggtgtggatcggcaactggaacagcccgtcggtgtcgcagcagcagatgcgtgacgccacgagccgcgcctcggccgccggcaagacggtgtcggtcgtcatctacgcgatcccgggccgcgactgcggctcgtactcgggcggcggcgtcgccgagagcgagtacgcgcagtggatcgacacggtcgcgagcggcatcgtcggcaagccgatcgtgatcctcgagccagacgcgctcgcccagctcggtgactgcggcggtcagggtgaccgcgtcggcttcctgcggtacgcggcgaagtcgctcacgcaggccggcggccgcgtctacatcgacgcgggccactccggctggctgtcggtggacaccgccgtgaaccgtctgaaccagatcggcttccagtacgcggtgggcttcgcgctcaacacgtcgaactaccagacgacgtccgcgtcgcagcagtacggcgagcagatctcgcagcgcctcggcggcaagggctacgtcatcgacacctcgcgcaacggcaacggctcgaacggcgagtggtgcaacccgcgcggtcgcgcgctcggcgacaagccgcgcctggtgaacgactcgacgggcctcgacgccctgctgtgggtcaagctgccgggtgagtcggacggcacgtgcaacggcggcccggccgccggccagtggtggcaggacatggccctggagctcgcgcgcaacgccaagtggtgacc

SEQ ID NO: 2 - protein, 457 aa, Cellulomonas uda
MSARRTASALLTAAALSVTGLTAVASTASAADGCRVDYTVQSQWNSGFQGDVKITNLGSPLSSWTVAWTFPSSGQRVSQSWNANVTTSGSRVSASSIGWNGSLGTGASTQFGFIGSFTDSNPVPTSFTVNGVTCTGTVTPTQGPTQGPTQGPTQGPTQGPTQGPTQGPTQGPTQNPQPSGDLYVDTENQSYAAWQAASGSTKDLLAKIALTPQSVWIGNWNSPSVSQQQMRDATSRASAAGKTVSVVIYAIPGRDCGSYSGGGVAESEYAQWIDTVASGIVGKPIVILEPDALAQLGDCGGQGDRVGFLRYAAKSLTQAGGRVYIDAGHSGWLSVDTAVNRLNQIGFQYAVGFALNTSNYQTTSASQQYGEQISQRLGGKGYVIDTSRNGNGSNGEWCNPRGRALGDKPRLVNDSTGLDALLWVKLPGESDGTCNGGPAAGQWWQDMALELARNAKW

SEQ ID NO: 3 - DNA, 2901 bp, Cellulomonas uda
  source 1..2901: mol_type = genomic DNA
atgcgtcgccgaacccagtccccctatccatcccgccgggtcaccggcacccaccgcggccgcgggcgcacccgcctcgccgccggtctgagcctcgccgtcgcggcgagcgcgctcgtcgtccccctggcgacgtccgcgagcgccgcccccgcgtacaactacgcggaggccctgcagaagtcgatgttcttctaccaggcccaggcgtccggcgacctgccggcgaactaccccgtctcctggcgcggcgactccggcatgtcggacggctccgacgtcggcgtcgacctcacgggcggctggtacgacgcgggtgaccacgtgaagttcggcttccccatggccttcacgaccacgatgctcgcgtggggcgccgtcgagtcccccgccggctacaccaagtcgggtcagctcgacgagctcaagggcaacctgaagtgggtcaacgactacttcatcaaggcccacaccaagcccaacgagctgtacgtgcaggtgggcggcggcgaggccgaccacaagtggtggggcccggccgaggtcatgacgatggcccgcccggcgtacaagatcaccgcgtcctgcccgggctccgacgccgcggccgagacggccgccgcgatggcgtcgtcgtcgctcgtcttcgcggactccgacccgtcgtacgcggcgacgctcgtcacgcacgccaagcagctgtactcgttcgcggacacgtaccgcggcgcgtactccgactgcgtcaaggacgcgcaggcgtactacaagtcgtggtcgggctaccaggacgagctcgtctggggcgcgtactggctgtacaaggccacgggtgacgcgacgtacctcgccaaggcggaggccgagtacgacaagctcggcaccgagaaccagtcgacgacgaagtccttcaagtggaccgtcgcgtgggacaacaagcagttcgcgacgtacgcgctgctcgccatggagaccggcaagcagaagtacgtcgacgacgcgaaccgctggctcgactactggaccgtcggcgccgaggggcagcggatcgcctactcgccgggcggcatggccgtgctcgactcgtggggcgcgctgcggtacgccgcgaacacctcgttcgtcgcgctcgtctactccgactggatcaccgactcgacgcgcaaggcccgctaccaggacttcggcgtcaagcagatcaactacgcgctgggcgacaacccgcgcaagtcgtcgtacgtcgtgggcttcggcaccaacccgccgacgatgccgcaccaccgcaccgcgcacggctcgtggctcgactcgatcacgacgcccgccgcctcgcgccacgtgctgtacggcgcgctcgtcggcggaccgtccgcggccaacgacgcgtacaaggacgaccggcaggactacgtcgccaacgaggtcgcgaccgactacaacgccggcttcacctcggcgctcgcctacctggtggaccggtacggcggcacggcgctgacgaacttccccgtggccgagacgcccgacggtgacgagatcttcgtcgaggcgcagctcaaccaggcccccggaagcacgttcaccgaggtcaaggccatgatccgcaaccagtcggcgttcccggcacggtcgctcaccaacggcaagctgcgctactggttccgcaccgacggcttcgccgcgtccgacgtgacgctcgcggccaactactccgagtgcggtccgcagtccggcaagggcgtctccgcgggcggcgacctggcgtacgtcgagctgtcgtgcgtcggccagaacatccacccgggcggccagtcgcagcaccgccgcgagatccagttccgcctcaccggcccggccgggtgggacgcgaccgacgacccgtcgttcaccggcctgacccagacgggcctcgccaagacgaagaacatcacgatgtacgacggcaccacgctcatctggggcaccgagcccacgggcacccaggtgccggacgacaccacggcacccacggcgcccggcctgccggtcgcctcggccgtgacgtcgaccagcgcgacgctcacctggcccgcgtcgagcgacgacacgggcgtcgtgggctacgacgtctaccgcgtccagggcacgacgagcacgctcgtcgcctccccgacggccgcgacgacgtcgctcacgggcctgaccgcgggcacggcgtacacgtactccgtcaaggcgcgtgacgccgcgggcaacgtctcggcgtcgtcgccgtcggtcacgttcacgacgacgaccgctccggccgacacgaccgcaccgaccgtcccgggcacgcccgcggcgaccgcggtcacgacgaccggcgcgctgctcacctgggccgcgtcgaccgactccggctccggcgtcgcgaagtacgaggtgctgcgggtgtccggcacgacgagcacgctcgtcggctcgcccaccacgccgtcgttcacgctcacggggctgacgcccgacacctcgtactcgtacgcggtgcgcgcggtcgacgccgcgggcaacacgtccgccgcctccacggccgtcacgttccggacgctcgaggagacgacgacgcccccgggcacgtgcaaggtcacgtacacgcccaacagctggaacaccggcttcaccgcctcgatcaaggtcgcgaacaccggcacgtcgcccctgacgtggtcgctcgcgttcgacctgaccgcgggccagaagctgtccagcggctggagcgcgacgtgggcgcagtcggggacgaccgtgaccgcgaccgggcagtcgtggaacgcgacgctggccccgggcgcgagcaccgagatcggcttccagggcacgcacaacggccagaacccgagcccgacgacgttcaccgtcaacggcacggcctgcagctga

SEQ ID NO: 4 - protein, 966 aa, Cellulomonas uda
MRRRTQSPYPSRRVTGTHRGRGRTRLAAGLSLAVAASALVVPLATSASAAPAYNYAEALQKSMFFYQAQASGDLPANYPVSWRGDSGMSDGSDVGVDLTGGWYDAGDHVKFGFPMAFTTTMLAWGAVESPAGYTKSGQLDELKGNLKWVNDYFIKAHTKPNELYVQVGGGEADHKWWGPAEVMTMARPAYKITASCPGSDAAAETAAAMASSSLVFADSDPSYAATLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQRIAYSPGGMAVLDSWGALRYAANTSFVALVYSDWITDSTRKARYQDFGVKQINYALGDNPRKSSYVVGFGTNPPTMPHHRTAHGSWLDSITTPAASRHVLYGALVGGPSAANDAYKDDRQDYVANEVATDYNAGFTSALAYLVDRYGGTALTNFPVAETPDGDEIFVEAQLNQAPGSTFTEVKAMIRNQSAFPARSLTNGKLRYWFRTDGFAASDVTLAANYSECGPQSGKGVSAGGDLAYVELSCVGQNIHPGGQSQHRREIQFRLTGPAGWDATDDPSFTGLTQTGLAKTKNITMYDGTTLIWGTEPTGTQVPDDTTAPTAPGLPVASAVTSTSATLTWPASSDDTGVVGYDVYRVQGTTSTLVASPTAATTSLTGLTAGTAYTYSVKARDAAGNVSASSPSVTFTTTTAPADTTAPTVPGTPAATAVTTTGALLTWAASTDSGSGVAKYEVLRVSGTTSTLVGSPTTPSFTLTGLTPDTSYSYAVRAVDAAGNTSAASTAVTFRTLEETTTPPGTCKVTYTPNSWNTGFTASIKVANTGTSPLTWSLAFDLTAGQKLSSGWSATWAQSGTTVTATGQSWNATLAPGASTEIGFQGTHNGQNPSPTTFTVNGTACS

SEQ ID NO: 5 - DNA, 3279 bp, Cellulomonas uda
  source 1..3279: mol_type = genomic DNA
atgcgtgctgacgtcgcgcttcgaaccggttcgggtgctcggaaccggttccgtacgcctggtttgcgctgtaccgtccccctggcgacgatcgagggtggtgtccggctctcgtggcagcgcccctgtggccatctggcccggcttcgaggagacgacgtggtttcctggtccagtcgccgtgcacgcacggctgtcctttctgcaggcctgacggccgcagccctggtcgccgcgacggtcgtcgcgccggccgccctcgcggatcccgtggggtcgacgcatgacttcgagtccggcgcgcagggctggcacagctacggcggcgactcgacgtcgtccgtcgtcgacggcgagttctgcgtcgtcgtccccacggggacgaaagacccgtggaccgtcgccgcgcagcacgacgggatcacgttcgaggcgggcaccacctacacggtgtccttcaccgcgcacgcgacgcaggacgtctcggtcaacctgcgcgcgggcatcgggtggcccgacgacgtggcgtcgaccgtcgccctgacctcgcaggagcaggagtactcgttcacctggaaccccgagttcagcggcgagggcaacctgtcgttccagctcggcgcgcaggcggccgactacacgttctgcctcgacgacttccagctcgcgggcgacgccgagctgatcccggacaccagcttcgacggtgtgctgcccgaggggtggagcgcggccggctggaccgtgacctccgagccgggcgagggggagccgctgtgcttcgaggtccccgcgggctcggggcagtacgacggcctcgtgctcaacggcctgccgatcgaggaggacgccaactaccgcctcacgttcacggcgagcgcgagcaacgcggcgacgatccgcacgatcgtcggcgagaacgccgtgccgtggcgcgcggcgttcgccgacaacacccagctgtcgaccgagctcacggagcacgagctcgcgttcacctcgcccgtgacgttcccggcgcagtccgacgacccggccgtcggtgtcgggcaggtcgcgctgcagatgggtgcccgcggggacttcacgttctgcatcacgagcctgtcgctcaagaaggtcgcgacgcccccgccgccgtacgagccggacgtgcgcggacccgtgcgcgtcaaccagctcggctacctgcccgacgggccgaagaacgccacggtcgtgagcgacgccaccgagccgctcgcgtggcggctcgaggactcgtcgggcgccaccgtcgcctcgggtgacgcgacgcccgccgggctcgacccctcgtcgcagctcaccgtgcagacgatcgacttctcgtcgttccgcacggccggcagcgggtacacgctcgtcgtgggcgaggaccgcagcgacccgttcgcgatcgcgaaggacctgtacgcgcagctgcgctacgacgcgctgaactacttctaccccgtgcgctcgggcatcgcggtggacgtccccgacgaccgctacgaccgtcccgccggccacgtgtccgggccggacggcgaggtcaacaagggcgacctcgacgtcgcgtgcctgaccgcggccgaggacggtgccgcgtggtcctacggcacctggacgtgcccgaccggctactcgctcgacgtcgtgggcgggtggtacgacgcaggtgaccacggcaagtacgtcgtcaacggcggcatcgccgtcgcgcagctgctgagcatgtacgagcggtcgctgcactcgcccgacgcggcccgcggcgccctcaaggacggcacgctcgacgtgccggccgacgagagcggcaacggcgtgccggacgtgctcgacgaggcccgctgggagctcgagttcttcctccagatgcaggtgccgaccggctccggcatgacggtgtccgcgaccgacaagcgctctctcgacggcctcgtgcaccacaagatccacgacgtcggctggacgggcctcggcctcctgccgtcgcaggacccgcaggagcgccgcctgcaccggccgtcgacggccgcgacgctcaacgtggcggcgaccgccgcgcagggcgcgcgactgttcgcgaagtacgacaaggcgttctcccagcagctcctgagcgtcgcgcgcagcacctacgccgccgcgaagcgggttcccgacctgtacgcgccgcagtccgcggggcagaacggtggcggcccgtacgacgacacggacgtcagcgacgagttctactgggcggccgccgagctctacctcacgacgggtgagaagacgttcgagaccgccgtgcagagcagcccgctcgcgacgagcgacatctgggcccgctccgggttctcctgggggagcgtcgcggcgctcggccggatggacctcgcgaccgtgcccagcaagctgccgacccgcaaggccgtgcaggcgtcggtcgtgaccggggccgagaagatcctcgcctggcagcaggccgaggcgttcggcacgacctacccgggcgacgccgacggctcgtacgtgtggggctcgagctcgtccgtgctgaacaaccaggtcgtgctcgccacggcgtacgacctcacgggtcaggcgcggttcgcgaacgcggtgctggagtcgatggactacctgctcgggcgcaacgcgctgaacatctcctacatcaccgggtacggcaccaagttcgcctcgcaccagcacagccggtggctcgtcccgccgccgccggggacgatcgcgggcggcccgaactcccagagcagcacgtgggacccggtcatgaaccggctctacccgccgggtcacgactgcgcgccccagctctgctacgtcgacgacatcgaggcgtggtcggtcaacgagctcaccatcaactggaacgccccgatgtcctgggtcgcgtcgttcgtcgccgacctcggcgtgagcggggtcccgaccgcgccggtcgtgaccaagcacccgtcggacgtctcggcgaagaagggcacgtccgtggcgctgcgggccaccgcgagcggcaagcccgcgccgtccgtgcagtggcaggtgcgtgacgccaagggtcgctgggtggacgtgcccggcgcccggacgacgaccctgcggatcgtcgtgacgccggcgaccgacggtgcgcagtaccgcgcggtgttccgcaacgcgtccggtaccgccacgaccgacgtcgcacgggtgacggtgctcgccggccccaagggccccggcccgaagggtccgggtcccatcccgtggcccggtcgcgggtga

SEQ ID NO: 6 - protein, 1092 aa, Cellulomonas uda
MRADVALRTGSGARNRFRTPGLRCTVPLATIEGGVRLSWQRPCGHLARLRGDDVVSWSSRRARTAVLSAGLTAAALVAATVVAPAALADPVGSTHDFESGAQGWHSYGGDSTSSVVDGEFCVVVPTGTKDPWTVAAQHDGITFEAGTTYTVSFTAHATQDVSVNLRAGIGWPDDVASTVALTSQEQEYSFTWNPEFSGEGNLSFQLGAQAADYTFCLDDFQLAGDAELIPDTSFDGVLPEGWSAAGWTVTSEPGEGEPLCFEVPAGSGQYDGLVLNGLPIEEDANYRLTFTASASNAATIRTIVGENAVPWRAAFADNTQLSTELTEHELAFTSPVTFPAQSDDPAVGVGQVALQMGARGDFTFCITSLSLKKVATPPPPYEPDVRGPVRVNQLGYLPDGPKNATVVSDATEPLAWRLEDSSGATVASGDATPAGLDPSSQLTVQTIDFSSFRTAGSGYTLVVGEDRSDPFAIAKDLYAQLRYDALNYFYPVRSGIAVDVPDDRYDRPAGHVSGPDGEVNKGDLDVACLTAAEDGAAWSYGTWTCPTGYSLDVVGGWYDAGDHGKYVVNGGIAVAQLLSMYERSLHSPDAARGALKDGTLDVPADESGNGVPDVLDEARWELEFFLQMQVPTGSGMTVSATDKRSLDGLVHHKIHDVGWTGLGLLPSQDPQERRLHRPSTAATLNVAATAAQGARLFAKYDKAFSQQLLSVARSTYAAAKRVPDLYAPQSAGQNGGGPYDDTDVSDEFYWAAAELYLTTGEKTFETAVQSSPLATSDIWARSGFSWGSVAALGRMDLATVPSKLPTRKAVQASVVTGAEKILAWQQAEAFGTTYPGDADGSYVWGSSSSVLNNQVVLATAYDLTGQARFANAVLESMDYLLGRNALNISYITGYGTKFASHQHSRWLVPPPPGTIAGGPNSQSSTWDPVMNRLYPPGHDCAPQLCYVDDIEAWSVNELTINWNAPMSWVASFVADLGVSGVPTAPVVTKHPSDVSAKKGTSVALRATASGKPAPSVQWQVRDAKGRWVDVPGARTTTLRIVVTPATDGAQYRAVFRNASGTATTDVARVTVLAGPKGPGPKGPGPIPWPGRG

SEQ ID NO: 7 - DNA, 1413 bp, Cellulomonas uda
  source 1..1413: mol_type = genomic DNA
atgaccccctcatccatgtccagacgcgcgcgcgtcgcgtccgcgctcgcgatcgtcacgctcggcgcgaccatcgcgacgacgatccccgcccaggccgccggcagcacgctgcaggccgcggcctccgagagcggccggtacttcggtaccgccatcgcggcgttcaagctcaacgacagcacgtactcgtccatcgcgaaccgtgagttcaacatgatcacggccgagaacgagatgaagatggacgcgacggagccgtcgcagaacaacttcagctactccagcggcgaccagatcctcaactgggcgcgcagcaacggcaagcgggttcgtgggcacgcgctcgcgtggcactcgcagcagccgggctggatgcagaacatgtccggcacccagctgcgcaacgcgatgctcaaccacgtcactcaggtcgcgacgcactacaagggcaagatctacgcctgggacgtcgtgaacgaggcgtacgcggacagcggcggcggccgtcgcgactcgaacctgcagcgcaccggcgacgactggatcgaggcggcgttccgcgcggcccgcgccgcggacccgggcgccaagctctgctacaacgactacaacacggacaactggacctgggccaagacgcagggcgtctacaacatggtcaaggacttcaaggcccgtggggtgccgatcgactgcgtcggtttccagtcacacttcaactcgggcagcccgtacccgagcaactaccggaccacgctgcagaacttcgccgcgctcggcgtcgaggtgcagatcaccgagctcgacatcgagggctcgggccagcagcaggcccagacgtacgccaacgtggtcgccgactgcctcgccgtgaaggcctgcaccggcatcacggtgtggggcgtgcgcgactccgactcgtggcgctcctcgggtaccccgctgctgttcgacggctcgggcaacaagaaggccgcgtacacctccacgctcgacgcgctgaaccgcggcggcgtcccgacggacccgacgacgccgcccacggacccgaccacgccgcccacggacccgacgaccccgcccacggacccgaccaccccgccgaccgaccccaccacgcctccgaccgacccgacgggccggtgcacggcgagcctggcgatcgcgaacgcgtggcccggcgggtaccaggcgaccgtgaccgtcaaggcgggctcgtcgtcgatcaacggctggcgcgtcacgctgcccagcggtgtcagcacgagcaacctctggaacggcgtgctcgccaacggtgtggtgaccaacgcgccgtacaacggctcggtcggcgccggccagtcgacgaccttcgggttcgtcggcaacggcagcgctccgagcgcaggctccgtgacctgcgcctga

SEQ ID NO: 8 - protein, 470 aa, Cellulomonas uda
MTPSSMSRRARVASALAIVTLGATIATTIPAQAAGSTLQAAASESGRYFGTAIAAFKLNDSTYSSIANREFNMITAENEMKMDATEPSQNNFSYSSGDQILNWARSNGKRVRGHALAWHSQQPGWMQNMSGTQLRNAMLNHVTQVATHYKGKIYAWDVVNEAYADSGGGRRDSNLQRTGDDWIEAAFRAARAADPGAKLCYNDYNTDNWTWAKTQGVYNMVKDFKARGVPIDCVGFQSHFNSGSPYPSNYRTTLQNFAALGVEVQITELDIEGSGQQQAQTYANVVADCLAVKACTGITVWGVRDSDSWRSSGTPLLFDGSGNKKAAYTSTLDALNRGGVPTDPTTPPTDPTTPPTDPTTPPTDPTTPPTDPTTPPTDPTGRCTASLAIANAWPGGYQATVTVKAGSSSINGWRVTLPSGVSTSNLWNGVLANGVVTNAPYNGSVGAGQSTTFGFVGNGSAPSAGSVTCA

SEQ ID NO: 9 - DNA, 2643 bp, Aspergillus niger
  source 1..2643: mol_type = genomic DNA
atgaggttcactttgatcgaggcggtggctctgactgccgtctcgctggccagcgctgatgaattggcctactcccctccgtattacccctccccttgggccaatggccagggtgactgggcggaagcataccagcgcgctgttgatatcgtctcgcagatgacattggctgagaaggtcaatttgactacgggaactggatgggaattggaattatgtgttggtcagactggaggtgttccccgattgggaattccgggaatgtgtgcacaggatagccctctgggtgttcgtgactccgactacaactctgcgttccccgccggtgtcaacgtggccgcaacctgggacaagaatctggcttacctgcgtggccaggctatgggtcaggagtttagtgacaagggtgctgatatccaattgggtccagctgccggccctctcggtagaagtcccgacggcggtcgtaactgggagggcttctcccccgacccggccctcagtggtgtgctctttgcagagacaatcaagggtattcaggatgctggtgtggttgcaacggctaagcactacatcgcctacgagcaggagcatttccgtcaggcgcctgaagctcaaggctacggattcaatattaccgagagtggaagcgcgaacctcgacgataagactatgcatgagctgtacctctggcccttcgcggatgccatccgtgcaggtgccggtgctgtgatgtgctcgtacaaccagatcaacaacagctatggctgccagaacagctacactctgaacaagctgctcaaggctgagctgggtttccagggctttgtcatgagtgattgggcggctcaccatgccggtgtgagtggtgctttggcgggattggacatgtctatgccgggagacgtcgattacgacagtggcacgtcttactggggtaccaacttgaccatcagtgtgctcaacgggacggtgccccaatggcgtgttgatgacatggctgtccgcatcatggccgcctactacaaggtcggccgtgaccgtctgtggactcctcccaacttcagctcatggaccagagatgaatacggcttcaagtactactatgtctcggagggaccgtatgagaaggtcaaccagttcgtgaatgtgcaacgcaaccatagcgagttgatccgccgtattggagcagacagcacggtgctcctcaagaacgatggcgctcttcccttgactggaaaggagcgcttggtcgcccttatcggagaagatgcgggttccaatccttatggtgccaacggctgcagtgaccgtgggtgcgacaatggaacattggcgatgggctggggaagtggcactgccaactttccctacttggtgacccccgagcaggccatctcgaacgaggtgctcaagaacaagaatggcgtattcactgcgaccgataactgggctattgatcagattgaggcgcttgctaagaccgccagtgtctctcttgtctttgtcaacgccgactctggtgagggttatatcaatgtcgacggaaacctgggtgaccgcaggaacctgaccctgtggaggaacggcgacaatgtgatcaaggctgctgctagcaactgcaacaacacgatcgttattattcactctgtcggcccagtcttggttaacgagtggtacgacaaccccaatgttaccgctattctctggggtggtcttcccggtcaggagtctggcaactccctcgccgacgtgctctacggccgtgtcaaccccggtgccaagtcgcccttcacctggggcaagactcgtgaggcctaccaagattacttgtacaccgagcccaacaacggcaacggagcgccccaggaagacttcgtcgagggcgtcttcattgactaccgcggatttgacaagcgcaacgagactcctatctatgagttcggctatggtctgagctacaccaccttcaactactcgaaccttcaggtggaggttctgagcgcccctgcgtacgagcctgcttcgggcgagactgaggcagcgccgactttcggagaggtcggaaatgcgtcggattacctctaccccgatggactgcagagaatcaccaagttcatctacccctggctcaacagtaccgatcttgaggcgtcttctggggatgctagctatgggcaggatgcctcagactatcttcccgagggagccaccgatggctctgcgcaaccgatcctgcctgccggtggtggtgctggcggcaaccctcgcctgtacgacgagctcatccgcgtgacggtgactatcaagaacaccggcaaggttgcgggtgatgaagttcctcaactggtaagtagacagcaaattcgaaccaagtcggaccaagctaatgaatcgcagtatgtttctcttggcggccctaacgaacccaagatcgtgctgcgtcaattcgagcgtatcacgctgcagccgtcggaagagacgcagtggagcacgactctgacgcgccgtgaccttgcgaactggaatgttgagacgcaggactgggagattacgtcgtatcccaagatggtgtttgtcggaagctcctcgcggaagctgccgctccgggcgtctctgcctactgttcactaagaattcgcc

SEQ ID NO: 10 - protein, 877 aa, Aspergillus niger
MRFTLIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVTVTIKNTGKVAGDEVPQLVSRQQIRTKSDQANESQYVSLGGPNEPKIVLRQFERITLQPSEETQWSTTLTRRDLANWNVETQDWEITSYPKMVFVGSSSRKLPLRASLPTVH

SEQ ID NO: 11 - DNA, 250 bp, Zymomonas mobilis
  source 1..250: mol_type = genomic DNA
tcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaac

SEQ ID NO: 12 - DNA, 250 bp, Zymomonas mobilis
  source 1..250: mol_type = genomic DNA
ataatcacttaatccagaaacgggcgtttagctttgtccatcatggttgtttatcgctcatgatcgcggcatgttctgatatttttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagtaagca

SEQ ID NO: 13 - DNA, 250 bp, Zymomonas mobilis
  source 1..250: mol_type = genomic DNA
gcgatggtgccgctggctaacatgttcggctatgtgaaccagctccgttccttcacccagggacgcgcccagtattcgatgcaattttcgcattatgacgaagtcccggctaacgtcgcggatgagttaaaatcgaagatggcttaattaaatactggcataaaccgaaaaatgtcgttatgagcgcgccggagaagcgcggcgcgctcaatacaatagtgataaaagcggtaacaaaaagaggtaacta

SEQ ID NO: 14 - DNA, 250 bp, Zymomonas mobilis
  source 1..250: mol_type = genomic DNA
aatcattggcagctgatgctacattcgccacagttttgttttcggccattgtctatactccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactcgtctaaaagattttaagaaaggtttcgat

SEQ ID NO: 15 - DNA, 250 bp, Zymomonas mobilis
  source 1..250: mol_type = genomic DNA
actatagaatataagttatgttccattcgcagaatagatatagatcagcctctatggatatgctatatatcgcccattccatttaagaataataataaaccatcatgctgtttatttaatatttttattacagtgaattgaagaaatattttcttgataaaaattattaaaaatctatcaccgacgatccgtctctatttcaagatagataataatttgtttaacctgttgattatgcgagataatttta

SEQ ID NO: 16 - DNA, 250 bp, Zymomonas mobilis
  source 1..250: mol_type = genomic DNA
gcagcattaagtatcttaggtggcttgattgttgctcgcttcgtgccggaaaccaaaggtcggagcctggatgaaatcgaggagatgtggcgctcccagaagtagttaaacttgctttggctgaatccttttgtcttttttagataagtcttaaccaattatactttttgtttacaacgatggtataaagcgggcggacaggctaaaaacaggctaaaaggattcggcctctgttttaaggacgagaata

SEQ ID NO: 17 - DNA, 250 bp, Zymomonas mobilis
  source 1..250: mol_type = genomic DNA
ttaaaaaataacttcattttactttaaattttccaagaaaatatttcgaaaatatttttgatatctttcttaattaagaaagaaaacttagttataatcctaccagttggacgaatcgcagacggtcgatttcgatttattcaaaaggccttttggcacagaagaaaaatcgaggtcatcgtcataatttaaagcgaatggacagcatatacctccgtattacggggggattttgtgagtggtgagaata

SEQ ID NO: 18 - DNA, 250 bp, Zymomonas mobilis
  source 1..250: mol_type = genomic DNA
aggcaccaacgcatcaagaacatcagccgccaatcggcctgctttccgcattccggcaaaagcgttttcatcgtaaagtttgatggcatgcgtacgaatagccgcatcaccttctttgacatggatatattcggtcataacttctgctataaccaataagctttccctttgcgagccgcattaacatattccgtttttggattcggaatatctgccgcatcagaggaaagaggaaaaggacggactgaaa

SEQ ID NO: 19 - DNA, 250 bp, Zymomonas mobilis
  source 1..250: mol_type = genomic DNA
gaacgggatcaatatcgctccgatccgatccagaaaatcaaacccttccatcagaatacctcggataaagttacccaattggaaaaactagccgcccttaaaaatcaaggcatcctgacagaagaagaatttgaaaaagaaaaaagacgtattttggccttataaaagacgaaaatatttcttacattgcccccatctcaaagacagtccgcctttcaaaatagatatcacaaaatcgggaaacagaatt

SEQ ID NO: 20 - DNA, 2634 bp, synthetic construct
  source 1..2634: mol_type = other DNA
atgagattcactttaatcgaagcggttgctcttactgccgtctctttagccagcgctgatgaattggcctattccccgccgtattatccctcaccttgggccaatggccagggtgactgggcagaagcatatcagcgcgctgttgatatagtctctcagatgacattggctgaaaaggtgaatttgacaacggggactggatgggaattggaattatgtgttggtcagacagggggtgttccccgattggggattccgggaatgtgtgcacaggatagccctttgggtgttcgtgattccgattataattctgcgtttccagccggtgtcaatgtggccgcaacctgggacaaaaatctggcttatctgcgcgggcaagcaatgggtcaggagtttagtgataaaggtgctgatatccaattgggtccagctgccggccctctgggtcggagtcccgatggcggtcgtaattgggaaggcttttccccggatccggccttatccggtgtgctgtttgcagaaacaattaagggtattcaagatgctggtgttgttgcaacggctaaacattatattgcctatgagcaggaacattttcgtcaggcgcctgaagctcaaggctatggattcaatattaccgagagtggaagcgcgaacttagacgataaaacgatgcatgagttatatttgtggccattcgcggatgccattcgtgcaggtgccggtgctgtgatgtgctcgtataatcagattaataactcatatggctgtcagaatagctacacactgaataagctgcttaaggcagaattaggtttccagggctttgtcatgagtgattgggcggctcatcatgccggtgtgtcaggtgctttggcagggttggacatgtctatgccgggggacgtcgattatgactcaggcacgtcttattggggtaccaatttgaccatctcagttctgaacgggacggttccgcaatggcgagttgatgatatggctgtacgaatcatggccgcctattacaaagtcggccgtgaccggctgtggacaccgccaaactttagctcatggaccagagatgaatatggcttcaaatattattatgtctcggaaggaccgtatgaaaaggtcaatcagtttgttaatgtgcaacgcaatcatagcgaattgatccgccgtattggagcagacagcacggttctgttgaaaaacgatggggctcttccgttgactggaaaagaacgcttggtcgcccttatcggggaagatgcgggttccaatccttatggtgccaacggctgcagtgatcgtgggtgtgataatggaacattggcgatgggctggggaagtggcacagccaattttccctacttggtgacccccgaacaggccatctccaatgaagttcttaagaataaaaatggcgtattcaccgcaaccgataattgggctattgatcaaattgaggcgttagctaaaaccgccagtgtatctcttgtctttgtcaatgccgactctggtgaaggttatattaatgttgatgggaacctgggtgatcgccgtaacctgaccttatggagaaatggcgataatgtcatcaaggctgctgctagcaactgcaacaatacgatcgttattattcactctgtcggcccagtcttagttaatgaatggtatgacaatcctaatgttaccgctattttatggggtggtttacctggtcaggaatctggcaattcccttgccgacgtgttatatggccgtgtcaacccaggtgccaaatcgcccttcacctggggcaaaactcgtgaagcctaccaagattatttgtataccgagcccaataacggcaacggagcgccacaggaagattttgtcgaaggcgtctttattgattatcgcggatttgataaacggaatgaaacacctatctatgaatttggctatggtctgagctataccaccttcaattattcgaaccttcaggtggaagttttaagcgcccctgcgtacgaacctgcttctggcgaaactgaggcagcgccgacttttggagaagtcggaaatgcgtcggattatctttaccccgatggtctgcaaagaatcaccaaattcatatatccctggttgaactcaaccgatcttgaggcatcttctggggatgctagctatgggcaggatgcctcagactatcttccagagggggccaccgatggctctgcgcaaccgattctgcctgccggtggtggtgctggcggcaatcctcgcctgtatgacgaacttatccgcgttacggttactattaaaaataccggcaaagttgcaggtgatgaagttcctcaactggtatccagacaacaaattcgaaccaaatcggatcaagctaatgaatcgcagtatgtttctcttggcggccctaatgaacccaaaatcgtgctgcgtcaattcgagcggatcacgctgcaaccgtcggaagaaacgcaatggagcacgactctgacgcgccgggatcttgcgaattggaatgttgaaacgcaggattgggaaattacgtcctatccgaagatggtgtttgtcggaagctcctcgcggaaattaccgcttcgggcgtctctgcctacagttcattaa

SEQ ID NO: 21 - DNA, 1365 bp, Neisseria sicca
  source 1..1365: mol_type = genomic DNA
atgtccgcccgcagaaccgcgtccgcgctgctgaccgccgcagcgttgtccgtcacaggcctcacggccgtacgcgagcaccgcgacgcagccgacggctgccgcgtcgactacaccgtccagagccagtggaactccgggttccagggcgacgtcaagatcaccaacctggggtccccgctctcgtcgtggaccgtcgcgtggacgttcccgtcctcgggccagcgcgtctcgcagtcgtggaacgcgaacgtcacgacgagcggctcgcgcgtctccgcgtcgagcatcggctggaacggctcgctcggcacgggtgcgtcgacccagttcggcttcatcggctcgttcaccgactcgaacccggtgccgacgtcgttcacggtcaacggcgtggcgtgcacgggcaccgtcaccccgacgcagggccccacgcagggtccgacccagggccccacgcagggcccgacccagggaccgacccagggtccgacccagggcccgacccagggccccacgcagaacccgcagccgtcgggggacctgtacgtcgacaccgagaaccagtcgtacgccgcctggcaggccgcgtccggcagcaccaaggacctcctggcgaagatcgcgctgaccccgcagtcggtgtggatcggcaactggaacagcccgtcggtgtcgcagcagcagatgcgtgacgccacgagccgcgcctccgccgccggcaagacggtgtcggtcgtcatctacgcgatccctgggcgcgactgcggctcgtactcgggcgggcgcgtcgccgagagcgagtacgcgcagtggatcgacacggtcgcgagcggcatcgtcggcaagccgatcgtgatcctcgagcccgacgcgctcgcccagctcggtaactgcggcggtcagggtgaccgcgtcggctatctgcggtacgcggcgaagtcgctcacagcagccggcggcgtctacatcgacgcgggccactccggctggctgtcggtggacaccgccgtgaaccgtctgaaccagatcggcttccagtacgcgctggcgttcgctctcaacacgtcgaactaccagacgacgtccgcgtcgcagcagtacggcgagcagatctcgcagcgcctcggcggcaagggctacgtcatcgacacctcgcgcaacggcaacggctcgaacggcgagtggtgcaaccgcggtcgcgcgctcggcgacaagccgcgcctggtgaacgactcgacgggcctcgacgccctgctgtgggtcaagctgccgggcgagtcggacggtacgtgcaagcggcccgccgccggccagtggtggcaggacatggccctggagtacgcgcgcaacgccaagtggtga

SEQ ID NO: 22 - DNA, 1350 bp, Cellulomonas sp. PSBB021
  source 1..1350: mol_type = genomic DNA
atgtccgcccgcagaaccgcgtccgcgctgctgaccgccgcagcgttgtccgtcacaggcctcacggccgtcgcgagcaccgcgagcgcagccgacggctgccgcgtcgactacaccgtccagagccagtggaactccgggttccagggcgacgtcaagatcaccaacctggggtccccgctctcgtcgtggaccgtcgcgtggacgttcccgtcctcggggcagcgcgtctcgcagtcgtggaacgcgaacgtcacgacgagcggctcgcgcgtctccgcgtcgagcatcggctggaacggctcgctcggcacgggtgcgtcgacccagttcggcttcatcggctcgttcaccgactcgaacccggtgccgacgtcgttcacggtcaacggcgtgacgtgcacgggcaccgtcaccccgacgcagggccccacgcagggtccgacccagggcccgacccagggcccgacgcagggaccgacccagggccccacgcagaacccgcagccgtcgggggacctgtacgtcgacacggagaaccagtcgtacgccgcctggcaggccgcgtccggcagcaccaaggacctcctggccaagatcgcgctgaccccccagtcggtgtggatcggcaactggaacagcccgtcggtgtcgcagcagcagatgcgtgacgccacgagccgcgcctcggccgccggcaagacggtctcggtcgtcatctacgcgatcccgggccgcgactgcggctcgtactcgggcggcggcgtcgccgagagcgagtacgcgcagtggatcgacacggtcgcgagcggcatcgtcggcaagccgatcgtgatcctcgagccggacgcgctcgcccagctcggtgactgcggcggtcagggtgaccgcgtcggcttcctgcggtacgcggcgaagtcgctcacgcaggccggcggccgcgtctacatcgacgcgggccactccggctggctgtcggtggacaccgcggtgaaccgtctgaaccagatcggcttccagtacgcggtgggcttcgcgctcaacacgtcgaactaccagacgacgtccgcgtcgcagcagtacggcgagcagatctctcagcgcctcggcggcaagggctacgtcatcgacacctcgcgcaacggcaacggctcgaacggcgagtggtgcaacccgcgcggtcgcgcgctcggcgacaagccgcgcctggtgaacgactcgacgggcctcgacgccctgctgtgggtcaagctgccgggtgagtcggacggcacgtgcaacggcggcccggccgccggccagtggtggcaggacatggccctggagctcgcgcgcaacgccaagtggtga

SEQ ID NO: 23 - DNA, 1371 bp, Cellulomonas palmilytica
  source 1..1371: mol_type = genomic DNA
atgtccgcccgcagaaccgtgtccgcgctgctgtccgtcgcggcgctgtccgtcgcaggcctcacggccgtcgcgagcaccgcgtccgccgccgaaggctgccgtgtcgactacaccgtccagagccagtggaacaccggcttccagggtgacgtgaagatcaccaacctggggtcgccgctgtcgtcgtggacggtcgcctggacgttcccgtcggggagccagaaggtgcagcaggcctggaacgcgaacgtcacgtcgagcggctcgcgcatctccgcgtcgagcgtgggctggaacggttcgctcggcaccggcgcgagcgcgcagttcggcttcatcggcgagtactccggcagcaaccccgtcccgtcgtcgttcacggtcaacggtgtcgcgtgcaccggcaccgtcccgacccaggagccgacgcaggagcccacccaggagcccacgcaggagccgacgcaggagccgacgcaggagcccactgaggagccgacgcaggagcccacgcaggacccgcagccgtccggcgacctgtacgtcgacgtcgagaaccagtcgtacgccgcctggcaggccgcgtccggcagcaccaaggacctcctggcgaagatcgcgctgacgccccagtcggtgtggatcggcaactggaacagcccgtccgtctcgcagcagcagatgcgtgacgcgacgagccgcgcgtcgcaggccggcaagacggtctcggtcgtcatctacgccatcccgggccgtgactgcggctcgcactcgggcggcggcgtcggcgagagcgagtacgcgcagtgggtcgacacggtcgcgcgcggcatcgtgggccgtccgatcgtcatcctcgagcccgacgccctcgcccagctgggtgactgcaacggtcagggcgaccgggtcggcttcctgaagtacgccgccaagtcgctcacgcaggccggcggccgcgtgtacatcgacgcgggccactcgggctggctgtcggtcgacaccgcggtgaaccgcctcaaccagatcggcttcgagtacgcggtgggcttcgcgctcaacacgtcgaactaccagacgacgtccgcgtcgcagcagtacggcgagcagatctcgcagcgcctcggcggcaagggctacgtcatcgacacgtcgcgcaacggcaacggcagcaacggcgagtggtgcaacccgcgcggccgtgcgctgggcgacaagccgcgtctcgtgaacgactcgacgggcctcgacgccctgctgtgggtcaagctgccgggcgagtccgacggctcctgcaacggcggcccggccgccggccagtggtggcaggagatcgccctggagctcgcgcgcaacgccaagtggtga

SEQ ID NO: 24 - DNA, 1404 bp, Cellulomonas xiejunii
  source 1..1404: mol_type = genomic DNA
atgcccccccgtacgaaggcggcccccgccgtcggacgcaagatcgtcgcggcgctcgccgcaagcgcggtcgtcgccgtcggcgtcaccgctctcacctccaccgcggcgaacgccgccgcaggttgccgcgcggactacaccgtcaccaaccagtggcccggcggcttcggcgcgtcggtcaccgtcaccaacctcggcgacccgctgtcgtcgtggaacctgcgctggacgttccccaacggccagagcatccagtcgctgtggaacggccaggtgtcgtcgagcggctcgcagatcacggtcaccaactcgccgtggaacggctcggtcggcaccaacggtgccatccaggtcgggttcaacggctcctggagcggcagcaacggcgccccgacgtcgttcacgctcaacggcacgacgtgcacgggctcggtcaccggcccgacgcagcagcccacccagcagcccacccagcagccgacgcagcagcccacgcagcagccgacgcagcagccgacgcagcagcccacgcagcagcccacgcagaacccgcagcccagcggggacttctacgtcgacccgaacaccgcggcgtacgccgcgtggcaggccgcgtcgggcaacgacaaggcgctcctggcgaagatcgcgcagacgccccaggcgctgtggatcggcgactgggcgaccgcctcggccacggcggagcaggtccgtgactacacgggccgtgcccgcgccgccggcaagacggcacagatcgtcgtctacgcgatcccgggccgggactgcggcaactactcgggtggcggcgtctcgacgtcggagtacgcgcgctgggtcgacaccgtcgcggcgggcatccagggcaacccgtgggtgatcctggagcccgacgcgctcgcgcagctcggtgactgcagcggccagggcgaccgggtcggcttcctgcagtacgcggccaagtcgctgaccgccaagggcgcccgcgtctacatcgacgcggggaactcggcgtggctctcggcgtcggaggcggcgaaccgcctcaaccgcatcgggttcagcgacgcggtcggcttctcgctgaacgtctcgaactaccggaccaccgcggaggccaaggcctacgggcagcagatctcccagctgaccggcggcaagaagttcgtcatcgacacctcgcgcaacggcaacggtgcgacgggcagcgagtggtgcaaccccagcggtcgcgcgctgggtgagcgtcccacgaaggtgaacgacggcagcgggctcgacgcgctgctgtggatcaagcgtcccggggagtcggacgggacgtgcaacggtggcccgtcggccggcgcctggtggcaggcgatggccctcgagctggcgcgcaacgccaagtggtga

SEQ ID NO: 25 - DNA, 1380 bp, Xylanimonas cellulosilytica
  source 1..1380: mol_type = genomic DNA
atgactacccgacgccatcgtctcgcgaccctggccgccgccgtctcgctggccgcggccggcgccgtcgtgaccgcgaccagcgccagcgcggctcccggctgcgccgtcacctatcaggtcgccaacgcgtggcagggcggcttccagggcgacgtgaccgtggagaacgtgggcgatgccctctcctcgtggaccctggagtggacggccgccgccgggcagggcgtggaccaggcgtggaacgccaccgtgtcgcagtccggcaaccaggtgaccgcgacgaacgtcgcctggaacgggtcgctcgccacgggggcgagcgcgtcgttcggcttcatcgggtcgggcagctcgacgccggtgccgacgtcgttcaccgtcaacggcgtgacgtgcacgggttcggtgacgacgccgaccccgacgcccactccgacacctactcccactccgacgccgacgccgacgccgacgccggaccccacgcctacccccacccccactcccactccgacgccgaccgtgccgccggtcgaggccggcgagctctacgtcgacccgacgacgcaggcgtacgccgcgtgggaggcggcatcgggcgccgacaaggcgctgctggagaagatcgccctgacgccccagtcctactgggtgggcaactgggcggacgccgcgcactcgaaggccgaggtcgtcgactacacctcccgggcggtcgccgcgggcaagaccggcatcctcaccgtctacgccatccccggccgcgactgcggcaaccactccggcggcggcgtcgagaccagcgagtacgccgcctggatcgacacggtggcgtcgggcatccagggccgcccgtacgtcgtcctcgagccggacgccctcccccagctcggtgactgcgacggccagggcgaccgcgtcggctacctgaagtacgcggccgagaagctgaccgccgcgggcggccgcgtgtacatcgacgccggtcactcggggtggatgggcgtgggcaccgccgtcgagcggctgaaccagatcgggttcgaccacgccgtcggcttcgcgctcaacacctcgaactaccagacgacggccgacagcatcagctacggcgagcagatctcgcagcagctcggcggacgcccgtacgtgatcgacacctcgcgcaacggcaacggctccaacggtgagtggtgcaacccgagcgggcgcgccctcggcgagaagccgcgggtcgtgaacgactccacgggcctcgacgccctgctgtggatcaagctgcccggcgagtccgacggttcgtgcaacggcggcccggccgccggccagtggttcggtgcgatggccctcgagctggcacgcaacgcggcgtggtga

SEQ ID NO: 26 - DNA, 1356 bp, Cellulomonas sp. H30R-01
  source 1..1356: mol_type = genomic DNA
atgtccacccgcagaaccgccgccgcgctcctcgcagccgcggccgtcgccgtcggcggtctcaccgccgtcacggccagcgccgcgcaggccgccgccggctgccgcgtgagctacgccgtgacgaaccagtggcaggacggcttcggcgccaacgtcaccatcaccaacctcggcgacccggtgtcgtcgtggaagctcgactggacctacggcgccggccagaagatcacgcagctctggaacggcaacgccacggtcaacggctcgcaggtctccgtgaccaacgtgtcgtggaacggccagatcggcacgggcggcaccgcgtcgttcggcttcaacggctcgtggagcggatcgaatccggtcccggcgtccttcagcctcaacggcacgacgtgcacgggcggcaccgtcccgacctcgacgccgacggtcaccccgacccagacgcccacgccgaccgtgacgcccacgccgacgcccaccccgacgacgacggtcccgccgcagccctcgggcagcttctacgtcgacacggccaaccagtcctaccgcgcgtggcagtcggcgtcgggcaccgacaagacgctgctcgcgaagatcgccctcacgccgcagtcgtactgggtcggcaactggaccgaggcgtcgaaggcgcaggccgaggtcgccgactacaccggccgtgccgtggccgcgggcaagacgccgacgctcgtcgtctacgcgatcccgggccgtgactgcggctcctactcgggcggcggcgtcgcggagtccgagtacgcgcgctggatcgacacgatcgcgcagggcatcaagggcaacccgatcgtcgtgctcgagcccgacgcgctcgcccagctcggcgactgctccggccagggcgaccgcatcggcttcctcaagtacgccgcgaagtccctcacgctgaagggcgcgaaggtctacatcgacgccggccactcgtcgtggctgtcggtcgacacggcggtcaaccgcctcaaccaggtcggcttcgagtacgccgtgggcttcgcgctcaacacgtcgaactaccagacgaccgccgcgagcaagtcctacggccagcagatctcgcagcgcctcggcggcaagggcttcgtcatcgacacctcgcgcaacggcaacggctcgaacggcgagtggtgcaacccgcgcggccgtgcgctcggcgagaagcccacgtacgtcaacgacggctccggcctcgtcgcgctgctgtgggtcaagctgcccggcgagtccgacggcacgtgcaacggcggcccggccgccggccagtggtggcaggacatggccctggagctggcgcgcaacgccaagtggtga

SEQ ID NO: 27 - DNA, 1476 bp, Cellulomonas flavigena
  source 1..1476: mol_type = genomic DNA
atgcacccccgctcgaagagacccctcaccaccagacgcaaggtcgtcgcggccgtcgcggccggagccgtcctcgccggcggcgtcaccgccctgacctcgagcatcgcgcaggccgccgccggctgccgcgtcgactacgccgtgacgagccagtggcccggtggcttcggtgcagccgtcaccgtcacgaacctcggcgacccgctctcgtcctgggagctgagctggacgttccccgacggccagggcgtgcagcagctctggaacggcgtgcactcgacctccggttcgaacgtcaccgtgaagaacatgtcgtggaacggttcggtcggcaccaacgccagcgtccaggtcggcttcaacggctcctggaacggcgcgaacaacgcgccgacgtccttcacgctcaacggcacctcgtgcaacggtgcggtcggtggcccgacgacggagccgacgcccgagccgaccccggagcccacgcccgagccgacgccggagccgacgcccgagccgacgccggagcccacgcccgagccgacgccggagcccacgcccgagccgaccccggagcccacgcccgagcccacgcccgagcccacgcccgagcccacgatgccgccggtccaggccggtcagttccacgtcgacaccacgaaccagtcgtaccgcgcctggcaggcggccagcggctccgacaaggacctgctggcgaagatcgccctgacgccgcaggcgtactgggtcggcaactggaacgaggcctcgcacgcgcagcaggaggtccgtgacatcacgtcggccgctgcggccgccggcaggaccgccgtgctcgtcgtctacgccatcccgggccgcgactgcggccagcactccagcggcggcgtgtcgacctccgagtacgcgcagtggatcgacacggtcgcccagggcatcgtcggcaacccgtgggtggtcctcgagcccgacgcgctgccgatgctcggcgactgcgacggccagggcgaccgggtcggcttcctcaagtacgccgcgaagtccctgaccgccaagggtgcgcgcgtctacatcgacgccggccactcggcgtggctgtcgccgtcggaggccgcgaaccgcctcaaccagatcgggttcgaggacgccgtgggcttctcgatcaacgtctccaactaccgcacgacggcggagtcgaagacctggggtcagcaggtctcgcagctgaccggtggcaagaagttcgtcatcgacacgtcgcgcaacggcaacggcccgtccgggtcggagtggtgcaacccgagcggccgcgccctcggcgagcgcccgacgctcgtgaacgacggcagcgggctcgacgcgctgctgtggatcaagctgcccggtgagtcggacggcgcctgcaacggcggcccgggcgccggtcagtggtggcagtcgatggcactggagctggcgcgcaacgcgaagtggtga

SEQ ID NO: 28 - protein, 454 aa, Neisseria sicca
MSARRTASALLTAAALSVTGLTAVREHRDAADGCRVDYTVQSQWNSGFQGDVKITNLGSPLSSWTVAWTFPSSGQRVSQSWNANVTTSGSRVSASSIGWNGSLGTGASTQFGFIGSFTDSNPVPTSFTVNGVACTGTVTPTQGPTQGPTQGPTQGPTQGPTQGPTQGPTQGPTQNPQPSGDLYVDTENQSYAAWQAASGSTKDLLAKIALTPQSVWIGNWNSPSVSQQQMRDATSRASAAGKTVSVVIYAIPGRDCGSYSGGRVAESEYAQWIDTVASGIVGKPIVILEPDALAQLGNCGGQGDRVGYLRYAAKSLTAAGGVYIDAGHSGWLSVDTAVNRLNQIGFQYALAFALNTSNYQTTSASQQYGEQISQRLGGKGYVIDTSRNGNGSNGEWCNRGRALGDKPRLVNDSTGLDALLWVKLPGESDGTCKRPAAGQWWQDMALEYARNAKW

SEQ ID NO: 29 - protein, 444 aa, Cellulomonas cellasea
MLSASLLAVTGIGAITATSAQAAAGCKVDYAVTNQWGGGFGANVTVTNLGDPVSSWTVDWAFPSGQSIQQLWNGVPSQSGAAVSVKNTTWNGSVATGGTASFGFNGSWNGANAVPTGFRLNGVACTGAPVSTPRPTQTSTPTPTPTVTTTPTPTPTVTPTTTPVPSGTFWVNPNTQAATAAAAASGETRRLLEKISTNAAGSWIGNWNSGTTSQQEVRTVTSAAAAKGQTALLIIYAIPGRDCGNHSAGGVATSEYAGWIDQVAAGIVGKPWIILEPDALAQLGDCDGQGDRVGYLKYAAQKLTQAGGRVYIDAGNSAWLSPSVAAQRLNQIGWDHAVGFALNTSNYRTTAESRSYGQQISQLTGGKTFVIDTSRNGNGANGTEWCNPSGRALGERPTLVNSGGLDALVWAKLPGESDGACNGGPAAGQWFQAQALELARNAKW

SEQ ID NO: 30 - protein, 468 aa, Paraoerskovia marina
MHRSPTIPPDAQPPRARHGVALVAATALLALAGTTTLAAVTLDADPAAAAGTCEADFDVNNQWDGGFGTNVAVTNTGDPVTSWEVTWEFSGDQQVTQLWNGTVSQSGNTVHVASLPWNGAVGTGGTLSFGFNGSGDGSTATPTAVTLNGAACSTGTDPTPTPTTTTPTPTPTETTPTPTPTETTDPPAADGDLYVDTGNQAYPAWSAASGDDKALLAKIALTPQAYWVGNWDDPEHAQAEVQDYTGRAVAAGQVPQLVIYAIPGRDCGSYSGGGVAESEYADWIDTMAAGIQGNPLVVLEPDALAQLGDCDGQGDRVGYLAYAASSLTDAGAHVYVDAGHSGWLAPSTAADRLLQVGLDDAVGFAVNTSNYQTTADSRAWAEQVSALTGGAGYVIDTSRNGNGSNGQWCNPSGRALGDRPSLVDDSTNLDALLWVKLPGESDGTCNGGPSAGAWWQSMALELARNASW

SEQ ID NO: 31 - protein, 469 aa, Cellulomonas avistercoris
MPPRTKAAPAAGRKLVAALAAGAVVAAGVTALTSTAASAAAGCRADYAISSQWPGGFGGTVTVTNLGDPLSSWDLRWSFPNGQTIQSLWNGQVSTSGSQVTVTNSPWNGSVGTNGTIQVGFNGSWSGSNGAPTSFTLNGTTCTGGVSGPTQQPTSTPSATPRPTQTPTQTPRPTQTPTPQPTQSQPPVSSSDFYVDPTTSAYAAWQAASGNDKNLLAKIALTPQALWIGDWATASATQEQVRDYTGRARAAGKTAQIVVYAIPGRDCGSYSGGGVSTSEYARWIDTVAAGIQGNPWVILEPDALAQLGDCNGQGDRVGFLQYAAKSLTAKGARVYIDAGNSAWLSASEAANRLNRIGFSDAVGFSLNVSNYRTTAEAKAYGQQISQLTGGKKFVIDTSRNGNGATGSEWCNPSGRALGDRPTKVNDGSGLDALLWVKRPGESDGTCNGGPSAGAWWQSMALELARNATW

SEQ ID NO: 32 - protein, 419 aa, Pseudoduganella lutea
MMHTVFRPALATLVLLASGAASAAPTCAATYATRDDWANGFVVDIVVTNTGSGTVSGWNVTWDYGSPVSLASAPWGARVAVAGGRITASDDGGRPTIEPGASVGFGLPLAYAGARPVPGRITVGGVNCTIQAPSPNLYSDPQSAAARWVRDNAGDGRAAEIGPRIASQPAAKWFGGWSGDIGNAVGSYVGAAAAAGRVPVLVAYNIPARDCGQHSVGGAGSAAAYQDWIRAFANAIGPREAVVVLEPDALPQLDCLEAAGKATRLQLFRHAVAQFNERAPRTALYIDIGNSDWLEPAEAASRLLQAGVANAEGFALNVSNYRTDAESNPYGIAVSNALQQQAGLGKPFVVDTSRNGAGPDGREWCDPAGRKIGVPPRVNAVGSQPAMTLWVKAPGEADGCAAAAGTFLPEMAYKMIHGY

SEQ ID NO: 33 - protein, 595 aa, Micromonosporaceae bacterium
MKLNLLRGGRHPRLRRALAASAALLAGGGMVVVAATQASAAAGCRVTYTVNQWNTGFTANLTVTNLGDPINGWTLRWTFSGNQQITQSWNSRFSQSGQQATLTNEAWNGNLGTNASTNPGFNASYSGTNEIPTSFTLNGTPCTGSVTGSPTGGPTSPTSGPTSPTSGPTSPTSGPTSNPPPGTHLDNPYVGARPYVNPDWSAKAAAEPGGSRIANQPTGIWMDRIAAITGTGGARGLREHMDTALQQGANLIQVVIYDLPGRDCSALASNGELGPDEIDRYKTEFIDPIAEIFADPAYRSLRIVAIIEIDSLPNLVTNAGGEAGATEMCRIMKENGNYVKGIQYALSKFHAIPNVYNYIDAAHHAWLGWDSNFGPAADLLASTAAGAVGGYDTVDGFIVNTANYSALTEPYFNINTTFNGVSVRQSKWVDWNQYVDELTFAQAFRQRLTGAFGNDIGMLIDTSRNGWGGPNRPTGPPTSASNIDDFVNQARIDRRIHAGNWCNQSGAGLGERPRATPAAGIDAYVWIKPPGESDGASQPIDNDEGKGFDQMCDPNYGGNARNGNNPTGALPNAPLSGHWFSAQFQELMQNAYPPL

SEQ ID NO: 34 - protein, 361 aa, Actinomycetales bacterium
MAANAARIGVGALAARLGTAAVTAVLLVGCGTTDTPAPSTPTGGQQPGRAAQNASPLAGPPAPGNPLVGRPFFVDGTGPAATQAAQWEADRAADAAELRKIADRPAAQWIGGEGDVTSKVDAVVGRAAAKGQLPLLVAYNIPHRDCGNFSAGGAGSSDEYRAWIHAFAAGVKGRPVIVVLEPDAVAHAVEGCNQEPDRLALLAEAVSVLKSTGKATVYIDAGNPRWITDVKRLATALQQAGVATADGFALNVANFITTTDNVTYGNAVSDALGGKTHFVVDTSRNGAGPVPGNGDINGGPRWCNPPDRLLGQAPTIETGQPRLDALLWVKRPGESDGACRPGEPEAGEWWPDYALSLAKRS

SEQ ID NO: 35 - protein, 361 aa, Cellulomonas chengniuliangii
MRRKTKIVAGAAAGALVMTVAVALVARSAAGAGAPTPAISAVTMSPSPGPTTAPALHPEEAPALVEPAPVKPAPAAGNPLAGVVFYVNPDTQAVRAAATAHGEAKQLLDKIAGTPQAFWVDDWNGLDSAREEVRGYTTRAVAAGAKPVVVVYAIPGRDCGWYSQGGIEHSGYAAWVDAIADSMVGNPWVILEPDALAQLGDCDGQGDRLGYLRYAAKSLTERAGARVYIEIGNPAWLTPGQAAAKLGLVGFEHAAGFAFNTSNFQPTDVSRAWGEAVSAEVGGKPYVIDVSRNGNGGDGADWCNPSGRALGERPRVVGEGGLDALLWVKLPGESDGPCNGGPAAGMWWQEGALELARNAHW

SEQ ID NO: 36 - protein, 456 aa, Cellulomonas palmilytica
MSARRTVSALLSVAALSVAGLTAVASTASAAEGCRVDYTVQSQWNTGFQGDVKITNLGSPLSSWTVAWTFPSGSQKVQQAWNANVTSSGSRISASSVGWNGSLGTGASAQFGFIGEYSGSNPVPSSFTVNGVACTGTVPTQEPTQEPTQEPTQEPTQEPTQEPTEEPTQEPTQDPQPSGDLYVDVENQSYAAWQAASGSTKDLLAKIALTPQSVWIGNWNSPSVSQQQMRDATSRASQAGKTVSVVIYAIPGRDCGSHSGGGVGESEYAQWVDTVARGIVGRPIVILEPDALAQLGDCNGQGDRVGFLKYAAKSLTQAGGRVYIDAGHSGWLSVDTAVNRLNQIGFEYAVGFALNTSNYQTTSASQQYGEQISQRLGGKGYVIDTSRNGNGSNGEWCNPRGRALGDKPRLVNDSTGLDALLWVKLPGESDGSCNGGPAAGQWWQEIALELARNAKW

SEQ ID NO: 37 - protein, 459 aa, Cellulomonas gelida
MTARRTSSRAAALLSAATLAIGGLTVAASSATAAEGCRVDYTVQSQWPGGFQGDVKITNLGSAISSWKVAWTWPASGQKVAQSWNADVQTSGSQVSASSVGWNGALGTGASASFGFIGAFTSANPAPTSFTVNGVACTGTVPTTGPTQNPTPTPTPTPTPTPTPTPTPTPTPTTTPPPQPSGDLYVDTTNQSYTAWQAASGSTKDLLAKIALTPQSVWIGNWNSPSVSQQQMRDATSRASSAGKTVSVVIYAIPGRDCGSHSGGGVPESEYAQWVDTVARGIVGRPIIVLEPDALAQLGDCSGQGDRVGFLKYAAKSLTQAGGRVYIDAGHSTWLSVDTAVNRLNQIGFEYAVGFALNTSNYQTTSSSQSYGEQISQRLGGKGYVIDTSRNGNGSNGEWCNPRGRALGDKPRLVNDSSGLDALLWVKLPGESDGSCNGGPAAGQWWQEIALELARNAKW

SEQ ID NO: 38 - protein, 467 aa, Cellulomonas xiejunii
MPPRTKAAPAVGRKIVAALAASAVVAVGVTALTSTAANAAAGCRADYTVTNQWPGGFGASVTVTNLGDPLSSWNLRWTFPNGQSIQSLWNGQVSSSGSQITVTNSPWNGSVGTNGAIQVGFNGSWSGSNGAPTSFTLNGTTCTGSVTGPTQQPTQQPTQQPTQQPTQQPTQQPTQQPTQQPTQNPQPSGDFYVDPNTAAYAAWQAASGNDKALLAKIAQTPQALWIGDWATASATAEQVRDYTGRARAAGKTAQIVVYAIPGRDCGNYSGGGVSTSEYARWVDTVAAGIQGNPWVILEPDALAQLGDCSGQGDRVGFLQYAAKSLTAKGARVYIDAGNSAWLSASEAANRLNRIGFSDAVGFSLNVSNYRTTAEAKAYGQQISQLTGGKKFVIDTSRNGNGATGSEWCNPSGRALGERPTKVNDGSGLDALLWIKRPGESDGTCNGGPSAGAWWQAMALELARNAKW

SEQ ID NO: 39 - protein, 450 aa, Cellulosimicrobium funkei
MRATSSTRRARAATFVAAVATLATLGATATAGAAIATSGAAAPSAASAACTVVYRITSSWSGGFQADVTVTNLGAARSGWEIAWDLAPGEGISQLWNGTLVRDGGRVTVGDVGWNASLATGGSASFGLVGTAASAPAVPTSFTLDGVACGGDAPPDPTDPPDPTDPPETPGDVTFHVDETNQAWEAWQSASGTDRDLLAKIALTPQSSWVTDADAQVSRAKVAAFTSAAAAEGATPLLTIYAIPGRDCGSHSGGGTAEAAYRSWVQTVASGIVGEPWVVLEPDALAQLGDCSGQGDRVGMLRDAAHILTDAGARVYVDAGHSAWLSPATAAARLQQVGLDHAVGFALNTSNYRTTAESRAYGEQVAALLGGDVSFVVDTSRNGNGSNGEWCNPRGRALGDQPRAVDDGTHLDALLWVKLPGESDGSCNGGPPAGQWWQEVALELARNASW

SEQ ID NO: 40 - protein, 452 aa, Demequina salsinemoris
MTRRVPALAVAIAIALSLLVTVAQPASAASAPCVAQYTVQSEWPGGFTAQVRIRNTSGKALTAWRAAFRFTDGQAISQVWNADTVRASGRAKVRNVSYNSHVAKGAWIDFGFNGTWSGKNTRPVAFALNGEKCKVRAWTATVSGGIATPGATASSGSEASTLAVGATTTVAQSSTKFFTSADTQAYAAYQSASGSQKRLLAKIARTPTARWIGDWYSASQAKAETRKYTKAAKRAGRTGVLVIYAIPGRDCGSYSAGGLTPKAYKRWIDAVANGIVGKPWIILEPDAIAQMGDCSGQGDRAGLLRYAARVLTKHGGRVYLDAGHSNWNSVSVTVRRLKQVGFRYAKGFALNTSNYNTTKAEKAYGQAISTKVGNRPFVIDTSRNGKGSNGQWCNPSGRGLGARPGLVKDSSNLKALLWIKAPGESDGSCNGGPSAGVWWQKGALTLARNASW

SEQ ID NO: 41 - protein, 451 aa, Glycomyces dulcitolivorans
MNRRQRLAGVVAAAATALAAALGAGFLLSPNAGAQDDVSAQAQALWTNPNTQAARWVAQNPNDSRTAMIRDRIAETPAGTWFTQTSPTGGIAAQVSAVVDGAAAAGAAPIMVVYNIPNRDCGGASGGGASSHSVYRGWIDQIAAQIDGPAYIVLEPDVIAHDCYGSSERAQINASLAYAAQTLNAADPGVKVYIDSGHSGWLDAPDAASRLQAAGIQYADGFSLNTSNYRTTAESTAYANQVRNIVGTNKGAVIDTSRNGNGPLGDQWCDPSGRAIGEHPTTATGVAGIDAYLWVKLPGEADGCAGAAGQFIPDIAYQLANNAGSGWPGDVEPTDPVTTPTGTGPTSEDPTTGGPSQGDCTVHIDVVSSWGSGWQGKVSMTTDDAVNGWTIGWTWPGSQSIQSSWNAQVTTSGSTVTAKDVGWNAAVGAGQTKELFGFVASGSAADFDVDC

SEQ ID NO: 42 - protein, 342 aa, Streptomyces lacrimifluminis
MVVLLVGGCCVGVLGTGEEDARTAGPGTGASVGSGEYAGGREGAGFEPLWVDPRSPAARQAAEWRLAGRDEDAELMDRIASRPQAAWVTGESPRAEVQTVTMAAARDGRTAVLVAYDIPGRDCGQYSGGGALGGLDYRTWVDGFAAGLGDRGAYVIVEPDAVAQVVAGCAQVDAEERYALLAYAVERIKRQPHARVYLDAGNSGWIPQPARLVEALRSAGVGQADGFALNVSNFQTDQDSSVYGDTLSRALGGKHYVVDTSRNGNGPYSGTDTGIGADAESWCNPPGRALGTPPTTVTGRPRLDAQLWIKRPGESDGTCRGGPPAGQWWSSYALELARNARS

SEQ ID NO: 43 - protein, 493 aa, Cryptosporangium aurantiacum
MTDAPRAGLLSRAGLRAGPYLARHRRGSANGRALTAGVGVAALCSVVVIGGTSLVAANATSEPDHSCAAAYTVRHTWSTGFSAEVAVTNTGEDPVKNWNVNWSFDGGKAVVKGWEGEWQQDDGLVTVRAGEDSVRLEPGKTLTLRFTGWHPQGKAPDAAQFALNGVSCQVGGDKEETPESASPTPSSIALPPGLPASPAPTGKPTKPAAGPLGSLFVDPSGQAVEWVEANSGDPRAAVIRERIAEQPQAHWLAANNPGEVESDVRGYTSRAHSRGKVPVLVAYNMTNRDCGGASAGGADSADGYRTWINNFADGLGSGTSIVILEPDALGHLTTCLSADQQQERLDLLEYAGKTIKQASPKARVFYDVGHSAWIDAGEAASRAKQAGAGQYGDGIALNTSNYNTSSSEVSYGKRVLASLGSGQMVVDTSRNGAGPAGGEWCDPAGRKLGKYPTTATGDPKVAAYLWVKRPGESDGCSGSAGSFIPETAYDLSS

SEQ ID NO: 44 - protein, 687 aa, Nocardiopsis lucentensis
MSTFRRVPDLGPAPRRGLAAMSVLLLGGALAAVTLPTTASAATQCEVDYQVANDWGSGFTANVEIANLGDPVDGWELSWDFDGDQQITNIWNATHTQSGPSVTAANVGHNATIGTGSGVSFGFQGTYGGDNPVPVEFTLNGTVCRGEVDPDPTGEPTDEPTDEPTDEPTDPDPNPGERVDNPYVGADVYVNPIWSANAAAEPGGDAVADEPTGVWLDRISAIEGNDSPTTGDYGLRDHLDEALAQAAGDPLVIQVVIYNLPGRDCAALASNGELGPDEIDRYKNEYIDPIADILADYEDTELRVVTTIEIDSLPNLVTNVGSRPTATPECDEMLANGNYVEGVGYALSTLGAIGNVYNYVDAGHHGWIGWDDNFTASAEVFHEAANAAGATPDDVHGFIANTANYSALEEPYIDVDGVIGGALVRQSAWVDWNRYADELSFAQALRTELVGVGFDENIGMLIDTSRNGWGGDERPTSPSSSTDVDTYVDESRMDRRIHIGNWCNQAGAGLGERPQAAPEPGIDAYVWMKPPGESDGSSEEIPNDEGKGFDRMCDPTYEGNPRNGHSMSGALPNAPVSGHWFPEQFQELLDNAYPPXXAPTRVGERGAAGEAAPRHLDSIRQRCLILWSEGDPCNPPEHRDSSSPSGRPSPSSRRSWFTHCPPRPTRAFVSRTAGWWTRRATTSSSAA

SEQ ID NO: 45 - protein, 449 aa, Cellulomonas sp. PSBB021
MSARRTASALLTAAALSVTGLTAVASTASAADGCRVDYTVQSQWNSGFQGDVKITNLGSPLSSWTVAWTFPSSGQRVSQSWNANVTTSGSRVSASSIGWNGSLGTGASTQFGFIGSFTDSNPVPTSFTVNGVTCTGTVTPTQGPTQGPTQGPTQGPTQGPTQGPTQNPQPSGDLYVDTENQSYAAWQAASGSTKDLLAKIALTPQSVWIGNWNSPSVSQQQMRDATSRASAAGKTVSVVIYAIPGRDCGSYSGGGVAESEYAQWIDTVASGIVGKPIVILEPDALAQLGDCGGQGDRVGFLRYAAKSLTQAGGRVYIDAGHSGWLSVDTAVNRLNQIGFQYAVGFALNTSNYQTTSASQQYGEQISQRLGGKGYVIDTSRNGNGSNGEWCNPRGRALGDKPRLVNDSTGLDALLWVKLPGESDGTCNGGPAAGQWWQDMALELARNAKW

SEQ ID NO: 46 - DNA, 2901 bp, Cellulomonas flavigena
  source 1..2901: mol_type = genomic DNA
atgcgtcgccgaaccgagtccccccatccgtcccgccgggtcaccggcacccaccgcggccgcgggcgcacccgcctcgccgccggtctgagcctcgccgtcgcggcgagcgcgctcgtcgtccccctggcgacgtccgcgagcgccgcccccgcgtacaactacgccgaggccctgcagaagtcgatgttcttctaccaggcccaggcgtccggcgacctgccggcgaactaccccgtctcctggcgcggcgactcaggcatgtcggacggctccgacgtcggcgtcggcctcacgggcggctggtacgacgcgggtgaccacgtgaagttcggcttcccgatggccttcacgacgacgatgctcgcgtggggcgccgtcgagtcccccgccggctacaccaagtcgggtcagctcgacgagctcaagggcaacctgaagtgggtcaacgactacttcatcaaggcccacaccaagcccaacgagctgtacgtgcaggtgggcggcggcgaggccgaccacaagtggtggggtccggccgaggtcatgacgatggcccgcccggcgtacaagatcacggcgtcctgcccgggctccgacgccgcggccgagacggccgccgcgatggcgtcgtcgtcgctcgtgttcgcagactccgacccgtcgtacgcggcgacgctcgtcacgcacgccaagcagctgtactcgttcgcggacacgtaccgcggcgcgtactccgactgcgtcaaggacgcgcaggcgtactacaagtcgtggtcgggctaccaggacgagctcgtctggggcgcgtactggctgtacaaggccacgggtgacgcgacgtacctcgccaaggcggaagccgagtacgacaagctcggcaccgagaaccagtcgacgacgaagtccttcaagtggaccgtcgcgtgggacaacaagcagttcgcgacgtacgcgctgctcgccatggagaccggcaagcagaagtacgtcgacgacgcgaaccgctggctcgactactggaccgtcggcgcggagggccagaggatcgcgtactcgccgggcggcatggccgtgctcgactcgtggggcgcgctgcggtacgccgcgaacacctcgttcgtcgcgctcgtctactccgactggatcaccgactcgacgcgcaaggcccgctaccaggacttcggcgtcaagcagatcaactacgcgctgggcgacaacccgcgcaagtcgtcgtacgtcgtgggcttcggcaccaacccgccgacgatgccgcaccaccgcaccgcgcacggctcgtggctcgactcgatcacgacgcccgccgcctcgcgccacgtgctgtacggcgcgctcgtcggcggcccgtccgcggccaacgacgcgtacaaggacgaccggcaggactacgtcgccaacgaggtcgcgaccgactacaacgccggcttcacctcggcgctcgcctacctggtggaccggtacggcggcacggcgctgacgaacttccccgtggccgagacgcccgacggtgacgagatcttcgtcgaggcgcagctcaaccaggcccccgggagcacgttcaccgaggtcaaggccatgatccgcaaccagtcggcgttcccggcacggtcgctcatgaacggcaagctgcgctactggttccgcaccgacggcttcgccgcgtccgacgtgacgctcgcggccaactactccgagtgcggtccgcagtccggcaagggcgcctccgcgggcggcgacctggcgtacgtcgagctgtcgtgcgtcggccagaacatccacccgggcggccagtcgcagcaccgccgcgagatccagttccgcctcaccggcccggccgggtgggacgcgaccgacgacccgtcgttcaccggcctgacccagacgggcctcgccaagacgaagaacatcacgatgtacgacggcaccacgctcatctggggcaccgagcccacgggcacccaggtgccggacgacaccacggcacccacggcgcccggcctgccggtcgcctcggccgtgacgtcgaccagcgcgacgctcacctggcccgcgtcgagcgacgacacgggcgtcgtgggctacgacgtctaccgcgtccagggcacgacgagcacgctcgtcgcctccccgacggccgcgacgacgtcgctcacgggcccgaccgcgggcacggcgcacacgtactccgtcaaggcgcgtgacgccgcgggcaacgtctcggcgtcgtcgccgtcggtcacgttcacgacgacgaccgctccggccgacacgaccgcaccgaccgtcccgggcacgcccgcggcgaccgcggtcacgacgaccggcgcgctgctcacctgggccgcgtcgaccgacgccggctccggcgtcgcgaagtacgaggtgctgcgggtgtccggcacgacgagcacgctcgtcggctcgcccaccacgccgtcgttcacgctcacggggctgacgcccgacacgtcgtactcgtacgcggtgcgcgcggtcgacgccgcgggcaacacgtccgccgcctccacggccgtcacgttccggacgctcgaggagacgacgacgcccccgggcacgtgcaaggtcacgtacacgcccaacagctggaacaccggcttcaccgcctcgatcaaggtcgcgaacaccggcacgtcgcccctgacgtggtcgctcgcgttcgacctgaccgcgggccagaagctgtccagcggctggagcgcgacgtgggcgcagtcggggacgaccgtgaccgcgaccgggcagtcgtggaacgcgacgctggccccgggcgcgagcaccgagatcggcttccagggcacgcacaacggccagaacccgagcccgacgacgttcaccgtcaacggcacggcctgcagctga

SEQ ID NO: 47 - DNA, 2901 bp, Cellulomonas palmilytica
  source 1..2901: mol_type = genomic DNA
atgcgtccgcgaacccagtccccccacccatccagcagagccacagccggccgacggacccgcgggcgcacccgcctcgccgccggcgtgagcctcgccgtcgcggccagcgcgctcgtcgtccccctggcgacgtccgcgagcgccgctcccgacttcaactacgccgaggcgctgcagaagtcgatgttcttctaccaggcccaggcgtccggcgacctgccggcgaactaccccgtctcgtggcgcggcgactccggcctgcaggacggctccgacgtcggcgtcgacctcacgggcggctggtacgacgcgggcgaccacgtgaagttcgggttcccgatggccttcacgacgaccatgctcgcgtggggtgccgtcgagtcccccggcggctacacgaagtccgggcagctggacgagctcaagggcaacctgaagtgggtcaacgactacttcatcaaggcccacaccaagcccaacgagctgtacgtgcaggtgggcggcggcgaggccgaccacaagtggtggggtccggccgaggtcatgacgatggcccgcccggcgtacaagatcaccgcgtcctgccccggctcggacgccgccgcggagaccgcggccgcgatggcgtcgtcgtcgctcgtgttcgccgactccgacccgtcgtacgcggccacgctcgtcacgcacgccaagcagctctactcgttcgccgacacctaccgcggcgcgtactccgactgcgtcaaggacgcgcaggcgtactacaagtcgtggtcgggctaccaggacgagctcgtgtggggcgcgtactggctgtacaaggccacgggtgacgcgacgtacctcgcgaaggccgaggccgagtacgacaagctcggcacggagaaccagtcgacgacgaagtccttcaagtggaccgtcgcgtgggacaacaagcagttcgcgacgtacgcgctgctcgccatggagacgggcaagcagaagtacgtcgacgacgcgaaccgctggctcgactactggaccgtcggtgccgagggccagaagatcgcgtactcgccgggcggcatggccgtgctcgactcgtggggtgcgctgcggtacgccgcgaacacctcgttcgtcgcgctcgtgtactccgactggatcaccgactcgacgcgcaaggcccgctaccaggacttcggcgtcaagcagatcaactacgcgctgggcgacaacccccgcaagtcgtcgtacgtcgtgggcttcggcgccaacccgccgaccgcgccgcaccaccgcaccgcgcacggctcgtggctcgactcgatcacgacccccgcggcctcgcgccacgtgctgtacggcgcgctcgtcggcggaccgtccgccgcgaacgacgcgtacaaggacgaccggcaggactacgtggccaacgaggtcgcgaccgactacaacgccggcttcacgtccgcgctcgcctacctggtcgaccggtacggcggcaccccgctggcgagcttccccgtggcggagacgccggacggcgacgagatcttcgtcgaggcgcagctcaaccaggcgcccggctcgacgttcaccgaggtcaaggccatgatccgcaaccagtcggcgttcccggcccggtcgctgaccaacggcaagctgcgctactggttccgcaccgacgggttcgcggcgtccgacgtgacgctcacggccaactactccgagtgcggctcgcagtccggcaagggcgtctccgcgggcggcgacctggcgtacgtcgagctgtcgtgcgccggccagaacatccaccccggcggccagtcgcagcaccggcgcgagctgcagttccggctcaccggccccaccggctgggacgcgaccgacgacccgtcgttcgccggcctgacccagacgggcctggccaagacgaagaacatcacgctgtacgacggcacgaagctcatctggggcaccgagccgaccggcacgcaggtcccggacgacaccacggccccgacggcgcccggcctgccggtcgcctcgtccgtgacgtcgacgagcgccacgctcacgtggtccgccgcgacggacgacacgggcgtcgtcgggtacgacgtctaccgcgtccagggcacgacgagcacgctcgtcgcctccccgacgaccaccacgacgtcgctcaccggcctgaccgccggcacggcgtacacgtacaacgtcaaggcgcgcgacgccgccgggaacgtgtcctcggcctcgccgtcggtcacgttcacgacgacgaccgcaccggccgacacgaccgcgccgaccgtcccgggcacgcccgcggcgtccgcggtcacgacgacgggcgcgaccctgtcctggaccgcgtcgaccgacgcgggctcgggcgtcgcgaagtacgaggtgctgcgggtctccggcacgacgacgacgctcgtcggctccccgacgacgccgacgttcacgctcacgggcctgacgccgaagacgtcgtacacgtacgcggtgcgtgcggtcgacgtcgccggcaacacgtccgccgcctcctcgtcggtcacgttcacgacgctcgaggagacgacgacacccccgggtgcgtgcaccgtcacgtacacgcccaacagctggaacacgggcttcaccgcctcggtgaagatcaccaactccggcacgtcggcgctcacgtgggcgctcgcgttcgacctcacggccgggcagaagctgagctccggctggagcgccgactgggcgcagtccgggaccaccgtcaccgcgaagggcttctcctggaacgcgacgctggctcccggtgcgagcaccgagatcggcttccagggcacgcacggcggccagaaccccagccccacgacgttcaccgtcaacggcgcggtctgcagctga

SEQ ID NO: 48 - DNA, 2886 bp, Cellulomonas gilvus
  source 1..2886: mol_type = genomic DNA
atgtcccccctgcaccttcccgcccgtgcgccgcggcgccgggcacgttcccgcagcgtcgcgggaaccgccggcctcgcgctggccgcaggtctcgtcgtcgcgccgatcgcggggagcgcccacgcggccgacccgtcgttcaactacgccgaggccctgcagaagtcgatgttcttctaccaggcccaggcctcgggcgatctgccggcgagctaccccgtctcgtggcgcggcgactccggcctgaccgacggcgcggacgtgggcaaggacctcacgggcggctggtacgacgcgggcgaccacgtgaagttcggcttcccgatggcgttcaccacgacgatgctcgcgtggggcgcggtcgcgagccccgacggctacgccaaggccgggcagctcgacgagctcgagggcaacctgcgctgggtcaacgactacttcatcaaggcccacaccgctcccaacgagctgtacgtccaggtgggcgacggcgaggcggaccacaagtggtggggccccgccgaggtcatgacgatggcgcgcccgtcgatgaagatcaccgcgtcgtgcccggggtcggacgtcgcgggggagaccgcggcggccatggccgcgtcctcgctcgtcttcgccgaggacgacccggcctacgcggcgaagctcgtcacgcacgccaagcagctgtactcgttcgcggacacctaccgcggcgcgtactccgactgcgtgaccggcgcggcggcgtactacaagtcctggtcgggctaccaggacgagctggtctggggtgcctactggctctacaaggcgacgggtgacgcggcgtacctggccaaggccgaggccgagtacgacaagctcggcaccgagaaccagtcgacgacgcggtcctacaagtggaccgtggcgtgggacaacaagcagttcgccacgtacgcgctgctcgcgatggagacgggcaagcagaagtacgtcgacgacgcgaaccgctggctcgactactggagcaccgggtacaacggcgagcggatccgctactcgccgggcggccaggcggtgctcgactcgtggggctcgctgcggtacgccgccaacacctcgttcgtcgcgctcgtctactcggactggatcaccgacccgacgcgaaaggcgcggtaccacgacttcggcgtgggtcagatcgactacgcgctcggtgacaacccgcgcggttcgtcgtacatggtgggcttcggcgtgaacccgccgaccaagccgcaccaccgcaccgcgcacggctcgtggctcgactcgagcgcgacgcccgtcgcgacgcggcacgtgctgtacggcgcgctcgtcggcggcccgagctcgccgaacgacgcgtacgtcgacgaccgccaggactacgtggccaacgaggtcgcgaccgactacaacgcgggcttcacgtctgcgctggcgtacctggtggaccagtacggcggcacgccgctcgcgagcttcccgaggcccgagacgcccgacggcgacgagatcttcgtcgaggcgcagctcaaccagcccccgggcggcacgttcagcgagatcaaggcgatgatccgcaaccagtcggcgttccccgcgcggtcgctcaagaacgccacggtccgctactggttccgcaccgacggcttcgcggcctcggacgtcacgctcaccgccaactactccgagtgcgggccgcagtccggcaagggcgtcagcgcgggcggcacgctgggctacgtgcagctcacgtgcgtgggccagaacatccaccccgggggccagtcgcagcaccggcgtgagctgcagttccggctcaccggcccggcgggctgggacgcgacgaacgacccgtcgttcgcgggcctgacctccacgggcctggccaagaccaaggcgatcacgctgtacgacggcgggacgctggtgtggggcaccgagccgaccggcaccacgccgggcgacaccacggcaccgaccgtcccgggcacgccggtcgcctccgccgtcacgtccacgggcgccacgctcacgtgggccgcctccaccgacgccggctcgggcgtcgcggcctacgacgtctaccgcgtccagggcacgacgagcacgctcgtcgcctccccgacggccgcgacgacgagcgtcaccgggctcacgccgggcacggcgtactcgtacgtcgtcacggcacgtgacgtggccgggaacgtctcggcggcctcggccgcggtgacgttcacgacgacggccgctcccgtggacacgacggcacccaccgtgccgggcacgccggtcgcgtcgggcatcacgtcctcgggtgcggcgctcacgtgggccgcctcgaccgacgcgggcaccggggtcgcgaagtacgaggtgctgcgggtcgacggctcgagcaccacgctcgtcgcatcgcccacggcacccacgaccacgctcacgggcctggccgcggacaccgcgtactcgtacgtggtgcgcgcggtggacggtgccggcaacgtgtccgcgaccagcacggcggtcacgttccggacgctcgccgggtccaccgacccggtcggcacgtgcgccgtcacctactcggccaacagctggaacaccggcttcacgggctcggtcaagatcacgaacaccggcacgtcgccgctcacgtggtcgctcggcttcacgttcccgtcgggccagaagatctcgcagggctggtccgcgacctggacgcagaccggtgccgtggtcacggccaccgggatgtcctggaacgccacgctcgcaccaggtgcgagcacggagatcggcttccagggcacgcactccggcaccaacacggcacccacgtccttcacggtcaacggcgccacctgcagctga

SEQ ID NO: 49 - DNA, 3165 bp, Cellulomonas sp. H30R-01
  source 1..3165: mol_type = genomic DNA
atgctcggccatgccacggcgccacgcgcgccccgatcccgcggtcgcaccgcgacggcgggagcctccgccctcgccgtcgccgccggactgctcgtcgccccgctcgcgacctccgcgagcgccgcccccgcgtacaactacgccgaggcgctccagaagtcgatgttcttctaccaggcgcagcgctccggcgacctgcccgacaacttccccgtctcgtggcgcggcgactccggcctcaccgacggcgccgacgtcggcaaggacctcaccggcggctggtacgacgccggcgaccacgtgaagttcgggctgccgatggcgttcagcgcgacgatgctcgcgtggggcgcgatcgacagccccggcgggtacacgaagtccggccagctcgacgagctcaaggacaacctgcgcttcgtcaacgactacttcgtcaaggcgcacaccgccccgaacgagctgtacgtgcaggtcggcgacggcgaggcggaccacaagtggtggggacccgccgaggtcatgacgatggcccgcccgtcgtacaaggtgtcggcgtcctgcccgggctccgacgtcgccgccgagaccgcggcctcgctcgcgtccgcgtcgatcctgttcaagggcgacgacccgacgtacgccgcgacgctgatctcgcacgcgaagcagctctattccttcgctgaccagtaccgcggcagctactccgactgcgtcaccgccgcctcggcgtactacaagtcgtggtccggctaccaggacgagctcgtctggggcgcctactggctctacaaggcgacgggtgacgcgtcgtacctgagcaaggcggagacggagtacgacaagctcggcaccgagaaccagagcaccacgaagtcctacaagtggaccgtggcgtgggacaacaagcagttcggcatgtacgcgctgctcgcgatggagaccggcaagcagaagtacgtcgaggacgcgaaccgctggctcgactactggaccgtcggcgtcaacggccagaaggtgccgtactcgcccggcggcatggccgtcctcgactcgtggggtgcgctgcggtacgccgccaacacgtcgttcgtcgcgctcgtctactccgactggctcaccgacgcgacgcgcaaggcgcggtaccacgacttcggcgtgcgccagatcaactacgcgctcggcgacaacccgcgcaagtcgtcgtacgtcgtcggcttcggcaccaacccgccgaccatgccgcaccaccgcaccgcgcacgggtcgtggctcgactcgatcaccacgcccgccgcatcacgccacgtgctgtacggcgcgctcgtcggcgggcccggctcgcccaacgacgcgtacaccgactcgcggcaggactacgtcgccaacgaggtcgcgaccgactacaacgcgggcttcacgagcgcgctcgcgcggctcgtcgacgagtacggcggcaccgcgctcgcgtcgttcccgacgcgtgaggcgcccgacggcgacgagatcttcgtcgaggcgatgctcaaccagcctcccggcaccacgtacaccgagatcaagggcatgatccgcaaccagtcggcgttcccggcccgctcgctcaagaacggcaagatccgctactggttcacgaccgacggcttcgccgcgtcggacgtcacgctgtcgtcgaactactcggagtgcggcgcgcagtccggcaagggcgtcagcgccggcgggaacctcggctacgtcgagctgtcctgcgtcggccagaacatccaccccggcggccagtcgcagcaccgccgggagatccagttccggctcaccggcccgacgggctggaacgcggcgaacgacccgtcgttcgcgggtctcggctcgacggccctcgcgaagacggccgcgatcacgctctacgacggctcgacgctgatctggggcaaggagccgaccggcaccacgaccgacacgaccgcaccgaccgtgccgggcacccccgtcgcgtcgggcatcacgaccgtcggcgcgtcgctcacctgggcggcgtcgaccgacgccgggtcgggcgtcgccgggtacgagctgtaccgggtccaggggtcgacgcagacgctcgtcgggaccaccaccgcggcggcctacatcctgcgtgacctcacgccgggcacgtcctactcctacgtcgtgaaggcgaaggacgtcgccggcaacgtgtccgccgcgtccgccgcggtcacgttcaccaccgagacgcagggcacgaccgagccgccgaccacgccgggcacgcccgtcgcgtccgcggtcacctcgaccggtgcgaccctcacgtgggccgcgtcgaccggcaccccggcggtcgccggctacgacgtcctgcgcgtccagggcaccaccgcgaccgtcgtcgcgcagaccaccgggacgacgcacacgctcaccggcctgacgcccggcaccgcgtacacctacgccgtgcgggcgaagaacaccgcgggcgacgtctccgcggtctccgcctcggtgacgttcacgacgaccgcaccgccggtcgacaccaccgcgccgaccgtccccgggacgcccgtcgcgtcgaacgtcagcacgacgggcgccacgctcacgtggaccgcgtcgaccgacaccggcggaagcggcctgtcgggctacgaggtgctgcgcgtcagcgggacgacgcagacgctcgtcgcgtcgccgaccaccgcgtcggtcgccctcaccggtctggcgcccgccaccgcgtacacctacgtggtccgggccaaggacggggccggcaacacgtccgcggtgtccgcggccgtcaccttcacgacgctgcccacgtcgacgaccgcctcgtgcaaggtcgcgtacacggcgaactcgtggaacaccggcttcaccggcgcggtgaagctcaccaacacgggcacgaagcccctgacctggaccctcgggttcaccttcccctctggccagcaggtcacgcagggctggagcgccacgtggtcgcagaccgggacgacggtcaccgcgacgggcgcgtcgtggaacgccacgctccagcctggtcagagcgtcgacatcgggttcaacggctcccacaccgggaccaacaccgacccgacgtcgttcaccgtgaacggtgaggtctgcggctga

SEQ ID NO: 50 - DNA, 3153 bp, Cellulomonas fengjieae
  source 1..3153: mol_type = genomic DNA
atgcaccgcccccgaactcgcgtcgcgatcgcgtgcagcaccgtgctcaccgtcgccgccggcctgctcgtcgccccgctcgcggggacggcgaccgccgcggagccggcgttcaactacgccgaggcgctccagaagtcgatgttcttctaccaggcgcagcgggcgggtgacctgcccgccgacaaccccgtctcgtggcggggcgactccgccctgaccgacggcgccgacgtcggcaaggacctcaccggcggctggtacgacgccggcgaccacgtgaagttcggcctgccgatggcgttcagctcgaccatgctcgcgtggggtgccatcgccgcaccgcagggctacacgaaggccggtcagctcgacgagctcaagggcaacctgcgctgggtcaatgactacttcgtcaaggcgcacaccgcacccaacgagctgtacgtgcagatcggcgacggcgaggccgaccacaagtggtggggccccgccgaggtcatgacgatggcgcggccgtcgtacaagatcagcccgtcctgcccgggctccgacgtcgcgggcgagacggcggcgtcgctggcctccgcgtcgatcgtgttcaagtccgaggacccgacgtacgcggcgacgctcctgtcgcacgccaagcagctctactcgttcgcggacacctaccgcggcaagtactccgactgtgtgaccgccgccgccgcgtactacaagtcgtggtccggctaccaggacgagctggtctggggcgcctactggctctacaaggcgaccggcgacgcgacctacctggccaaggcggaggccgagtacaccaagctcagcgtcgagaaccagacgaccacccggtcctacaagtggaccgtcgcgtgggacgacaagagctacgcgatgtacccgctgctggccatggagaccggcaagcagcagtacgtcgacgacgccaaccggtggctcgactactggaccgtcggcgtcaacgggcagcggatcccctactcgcccggcggccaggccgtcctcgactcgtggggctcgctgcggtacgcggcgaacacctcgttcgtcgcgctggtgtactcggactggctgaccgacaccacccgcaaggcccggtaccacgacttcggcgtccggcagatcaactacgcgctcggcgataacccgcgcaagtccagctacgtggtcggcttcggggccaacccgccgcagaacccgcaccaccgcaccgcgcacggctcgtggctcgactcgctgacgcagccggagaagacccgtcacgtcctgtacggcgcgctcgtcggcggacccagcggtccgaacgacgcctacaccgacctgcggtccgactacacggccaacgaggtcgccacggactacaacgccgggttctccagcgcgctcgcgcggctgacccaggagtacggcggcaccccgctggccggcttccccgtcgccgagcggccggacggcgacgagatcttcgtggaggccatgctcaaccagccggccggcggcacgttcaccgaggtcaaggcgatgatccgcaaccagtcggcgttccccgcgcgggcgctcaagaacggcacgctgcgctactggttcacgctcgacgccggtgactcggccgccaacctcaccctctcggccaactacagcgagtgcggcccgcagacggccacgccggtcagcgcaggcggggggctctactacgccgagctcacctgcgtggggcagaacatccaccccggcggtcagtcgcagcaccggcgggagatccagttccgggtcagcggtggttccacgtggaacgccgcgaacgacccgtcgttcgcgggtctgagcaccgccggtctggccaagacgaaggcgatcaccctctacgacggcggccggctcatctggggcgccgagccgacgggcacggtcaccgacacgaccgcgccgaccgtgccgagcaccccggtcgcggccggcatcacctcgaccggtgcctcgctgagctggacgccgtccaccgatgcgggcagcggtgtcgcggggtacgaggtctaccgcgtccaggggaccacctcgacgctggtcgcctcgaccaccagcccgtcgacggccctgtccggcctgacgccgagcaccgcgtactcctacacggtccgggccaaggacgtggccggcaacgtgtccgccgcctcggcggcgggcacgttcaccacggcggagtcgcccgtcgaggtcgtcccgcccggcacacccggcacgcccgtggcctcagcggtgacggcgaccggcgccacactgacctggacgccgtcagcggccggcacctacgcggtggccgggtacgacgtgctccgggtcaccgggaccacgtcgagcgtcgtcggcacggcgaccggcacgacgttcgccgtgaccggcctgacggcggggacggcgtacacgtacgcggtgcgcgccagggacgcggccgggaccacctcgcccgtgtccgcggcggtcacgttcaccacgggcgcggccacggacaccacggctcccagcgtgcccggcaccccggtcgcctcgtcgatcacccagaccggcgcgacgctcacctgggccgcgtcgaccgacgcggggggcagcgggctcgcggggtacgacgtgctgcgggtcaacggcaccacctcgacggtcgtcggcaccgcgacgggcaccacgttcgcgctcaccggtctcacggcgaacaccgcgtacacgtacgcggtgcgcgccaaggacgccgccggcaacgtgtccgccgccggcacaccggtcggcttctcgacgctgccggcgacgtccaccagcagctgcgccgtgacctactcgaccaacagctggaacgtcggcttcacgggcagcgtgcggatcacgaacacgtcgaccgcgccgctgacctggacgctcgggttcgcctttccctccggtcagcgcctgacccaggggtggagcgcgacgtgggcccagacggggtccgccgtcacggccaccggcctcgggtggaacacgacgctggcaccgggggcgagcacggacatcgggttcaacggctcgcacgccgggaccaacaccgcgccgaccgccttcactgtcaacggcacggcctgcacggtctcctga

SEQ ID NO: 51 - DNA, 2397 bp, Cellulomonas sp. JZ18
  source 1..2397: mol_type = genomic DNA
atggcccgtcaggcacgcgccacgcgcccgtggtgggcgaccgccgccgccctggccctcgccgccggcgcggtcaccgtgccgctgtcggccgcgagcgcggcacccgcgtacaactacgcggaggcgctgcagaagtcgatgttcttctaccaggcgcagcgctcgggcgacctgccggaggacttcccggtctcgtggcgcggcgactcgggcctgcgcgacggcgcggacgtgggggaggacctgaccggcgggtggtacgacgccggggaccacgtgaagttcggcctgcccatggcgttcaccgccacgatgctcgcgtggggcgcgatcgagagcccggaggggtaccgcaaggccgggcagctcgacgagctcaaggacaacctgcgctgggtcaacgactacttcgtcaaggcgcacacggccccgaacgagctgtacgtgcaggtcggcaagggcgacgacgaccacaagtggtggggcccggccgaggtgatgacgatggcccggccggcgtacaggatcaccgcgtcctgcccgggctcggacgtcgccgccgagaccgcggccgcgatggcctccgcctcggtgctgttcgccgaggacgaccccgcctacgcgcgcacgctcgtctcgcacgcgaagcagctctactccttcgccgacacgtaccggggcaactactccgactgcgtcaccgacgcgcagtcgttctaccgctcgtggtccggctaccaggacgagctcgtctggggcgcgtactggctgtacgaggcgacgggcgacgcgtcctacctggcgaaggcggaggcggagtacgacaagctcggcaccgagaaccagacgacgacgaagaagtacaagtggacggtcgcctgggacgacaagtcgttcggcgcgtacgccctgctggcgatggagacggggaagcagaagtacgtcgacgacgcgaaccgctggctggactactggacggtcggcgtgaacggccagaaggtgccgtactcgccgggcggcatggccgtgctcgactcgtggggcgcgctgcggtacgccgcgaacacgtcgttcgtggcgctcgtgtactcggactggatcaccgacgcgacgcgcaaggcgcggtaccacgacttcggcgtccggcagatccactacgcgctcggcgacaacccgcgcaggtcgtcctacgtggtcgggttcggcgcgaacccgccgcagaacccgcaccaccgcaccgcgcacggctcgtggctggactcgctcacgcagccggccgagacgcggcacgtgctctacggcgccctcgtcggcgggcccggctcgccgaacgacgcgtacaccgacagccggtccgactacgtggcgaacgaggtcgcgaccgactacaacgccggcttcacgagcgcgctcgcgcgcctcaccgaggagtacggcggcaccccgctggcgtcgttcccgcgccccgagacgcccgaccaggacgagctgttcgtcgaggcgcggctcaaccagccggccggtggcacgttcaccgaggtgaaggcggtcatccggaaccggtccgccttcccggcgcggtcgctgcgcgacgcgtcggtccggtactggttcacgctcgacgggtccgacccggcgtcggggttgaccgtctccaccaactacagcgagtgcggcacgcagacgaacgcggtgaagcacgccggcggggacctgtactacgccgagctgcggtgcgcgggccaggacatccacccgggcgggcagtcgcagcaccgccgcgagatccagttccgggtcagcgggggagcggcgtgggacgcgacggacgacccgtcgtcggcgggcctcgtcgcgaccggcgaccccgtgaagacccgtgcgatcacgctgtacgaggccggccgtctggtctggggcaccgagcccgacgggacgtcgaccccgacccccacgccgacggtgacgcccaccgcgacccccaccgccaccgccacggccacccccaccgcgaccgcgaccgcgacgccgaccgttaccgccaccccgacgcccacgcagacgtcgagcccgacgccgaccgggtgcgccgtgcggtactccgcgaacgggtggagcaccggcttcacggcgaccgtgcggctgacgaacaccggcaccgcacccctgacgtggtcgctcgcgttcgacctgaccgccgggcagcggctgcagcagggctggagcgcgacgtggagccagagcgggacgcgcgtgaccgccaccggcgccgcgtggaacacgacactcgcccccggggcgtccgtggagctcgggttcaacgggtcgcacaccggccagaacccggccccgacggcgttcacggtcaacgggacggcctgcaccacgggatga

SEQ ID NO: 52 - protein, 966 aa, Cellulomonas flavigena
MRRRTESPHPSRRVTGTHRGRGRTRLAAGLSLAVAASALVVPLATSASAAPAYNYAEALQKSMFFYQAQASGDLPANYPVSWRGDSGMSDGSDVGVGLTGGWYDAGDHVKFGFPMAFTTTMLAWGAVESPAGYTKSGQLDELKGNLKWVNDYFIKAHTKPNELYVQVGGGEADHKWWGPAEVMTMARPAYKITASCPGSDAAAETAAAMASSSLVFADSDPSYAATLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQRIAYSPGGMAVLDSWGALRYAANTSFVALVYSDWITDSTRKARYQDFGVKQINYALGDNPRKSSYVVGFGTNPPTMPHHRTAHGSWLDSITTPAASRHVLYGALVGGPSAANDAYKDDRQDYVANEVATDYNAGFTSALAYLVDRYGGTALTNFPVAETPDGDEIFVEAQLNQAPGSTFTEVKAMIRNQSAFPARSLMNGKLRYWFRTDGFAASDVTLAANYSECGPQSGKGASAGGDLAYVELSCVGQNIHPGGQSQHRREIQFRLTGPAGWDATDDPSFTGLTQTGLAKTKNITMYDGTTLIWGTEPTGTQVPDDTTAPTAPGLPVASAVTSTSATLTWPASSDDTGVVGYDVYRVQGTTSTLVASPTAATTSLTGPTAGTAHTYSVKARDAAGNVSASSPSVTFTTTTAPADTTAPTVPGTPAATAVTTTGALLTWAASTDAGSGVAKYEVLRVSGTTSTLVGSPTTPSFTLTGLTPDTSYSYAVRAVDAAGNTSAASTAVTFRTLEETTTPPGTCKVTYTPNSWNTGFTASIKVANTGTSPLTWSLAFDLTAGQKLSSGWSATWAQSGTTVTATGQSWNATLAPGASTEIGFQGTHNGQNPSPTTFTVNGTACS

SEQ ID NO: 53 - protein, 966 aa, Cellulomonas palmilytica
MRPRTQSPHPSSRATAGRRTRGRTRLAAGVSLAVAASALVVPLATSASAAPDFNYAEALQKSMFFYQAQASGDLPANYPVSWRGDSGLQDGSDVGVDLTGGWYDAGDHVKFGFPMAFTTTMLAWGAVESPGGYTKSGQLDELKGNLKWVNDYFIKAHTKPNELYVQVGGGEADHKWWGPAEVMTMARPAYKITASCPGSDAAAETAAAMASSSLVFADSDPSYAATLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQKIAYSPGGMAVLDSWGALRYAANTSFVALVYSDWITDSTRKARYQDFGVKQINYALGDNPRKSSYVVGFGANPPTAPHHRTAHGSWLDSITTPAASRHVLYGALVGGPSAANDAYKDDRQDYVANEVATDYNAGFTSALAYLVDRYGGTPLASFPVAETPDGDEIFVEAQLNQAPGSTFTEVKAMIRNQSAFPARSLTNGKLRYWFRTDGFAASDVTLTANYSECGSQSGKGVSAGGDLAYVELSCAGQNIHPGGQSQHRRELQFRLTGPTGWDATDDPSFAGLTQTGLAKTKNITLYDGTKLIWGTEPTGTQVPDDTTAPTAPGLPVASSVTSTSATLTWSAATDDTGVVGYDVYRVQGTTSTLVASPTTTTTSLTGLTAGTAYTYNVKARDAAGNVSSASPSVTFTTTTAPADTTAPTVPGTPAASAVTTTGATLSWTASTDAGSGVAKYEVLRVSGTTTTLVGSPTTPTFTLTGLTPKTSYTYAVRAVDVAGNTSAASSSVTFTTLEETTTPPGACTVTYTPNSWNTGFTASVKITNSGTSALTWALAFDLTAGQKLSSGWSADWAQSGTTVTAKGFSWNATLAPGASTEIGFQGTHGGQNPSPTTFTVNGAVCS

SEQ ID NO: 54 - protein, 898 aa, Micromonospora sp. BL4
MRHHTRPVPGRYRPPVTDQPWPRRLLAADQSWPRRLLATTDQPWPRRLLATTDQPWPRRLLAAGVALLIGLALAVAAPPSGPASAAPAAFNYAEALQKSLLFYEAQQSGRLPDWNRVSWRGDSALTDGADAGLDLTGGWYDAGDHVKFGFPMAFSATMLAWGAVEYRSGYTASGQLPHLLNNLRFVNDYFIRAHPSANVLYGQVGKGDDDHKWWGPAEVLPMARPAYKIDASCGGADLAGETAAAMAASSMVFRPTDAAYADRLLGHARQLYTFADTVRKSYHECITDATSFYRSWSGWQDELVWAAIWLHRATGEASYLAKAESEYDKLGTENQSTTRSYKWTIAWDNKQFGAYVLLANLTGKQKYVDDANRWLDYWTVGVDGQRVPYSPGGMAVLDSWGALRYAANTSFAALVYSDRTTDTTRKARYHDFAVRQINYALGDNPRNSSYVIGFGANAPRNPHHRTAHGSWWDSQTVPVETRHTLYGALVGGPSAANDAYTDSRSDYVMNEVATDYNAGFTSALVRLTSEYGGTPLANFPVAETPDLDELTVETTVMQAEPRATGLKVMIYNKSAFPARALTDGRFRYYFRPDGTGGVQVTAGYTQGCPSPTTARQLSGDIWYVEVDCTGHTIAPAGQSQHRMEVQFKIGVPEGGTWDPTNDPSYQATAGPNRKVPLYSGATRVWGDEPGPVVPDTTAPSPPGTPVASAVTATGLTLSWAASTDNVGVTGYRVHREAGATDPLVGSSTGATLAVSGLTASTAYQFYVVAVDAAGNTSAASAPVTVTTAPPPVTGACTVGYATNDWSTGFTAAVTITNTGTTAINGWTLRFSFPGGQTVSQGWSATVTQAGTAVTATGLSYNGTLAPGASVSFGFNGTHTGSNPRPTAFTVNGVACALG

SEQ ID NO: 55 - protein, 964 aa, Asanoa ferruginea
MHHVRRLRRARSDQPWSRRVLAAGAALVTGLTLGAVPGPASAAVPATTGVQAAAAAFNYAEALQKSLFFYEAQQSGPKPSWNRVSWRGDSALTDGADVGLDLTGGWFDAGDHVKFGFPMAFSATMLAWGAVEYRDGYVSSGQLPHLLNNLRFVNDYFVKAHPSPNVLYGQIGNGDADHKWWGPAEVMPMARPSYKIDASCGGADLAGETAAAMAASSMVFRPTDAAYADKLLGHAKQLYTFADTVRKSYHDCITDATNFYRSWSGYQDELVWGAIWLYRATGDASYLAKAESGYDLLGNENQTTTKSYKWTIAWDNKQFGAYVLLARLTGKQKYLDDANRWLDYWTVGVNGQRVPYSPGGMAVLDSWGALRYAANTSFVALLHGDYLTDATRKARYQDFALRQINYALGDNPRNSSYVIGFGANSPKNPHHRTAHGSWWDSQTVPEETRHVLYGALVGGPSSANDAYSDTRSDYVMNEVATDYNAGFTSALAALVTKHGGTPLANFPVAETPDRDELTVETTVMQAEPRATGLKAIIYNRSAYPARALTDAKFRYYFRAEGATPVVVTPGYTQGCPSPSTARQVSGDLWYVEVDCTGYTIAPAGQSQHRMEVQFKVGVPEGGTWDPTNDPSYSTVTGPNRNVPLYAGGRRVWGVEPGAPTPDTTAPTAPGRPTASAVTATGATLSWAASTDNVGVTGYDVYREAGATDVLVGSPTAATFAVTGLTAATSYAFYVVAKDAAGNRSAASAPVSVTTTDPPAPDAVAPTAPGQPTASTITTSGVTLSWAASTDNVGVTGYRVYRENGATDLLVGSPTGTTLAVTGLTAATAYTFYVVAVDAAANTSPPSASVTVTTAAPPAAGACTVAYATNDWGSGFTANITITNTGTTTINGWSLGFTFPHSGQKVGQGWSATFTQTGTAVTATNLSYNGALAAGASTGIGFNGTHTGSNPKPTSFTLNGVACTA

SEQ ID NO: 56 - protein, 965 aa, Micromonospora profundi
MRHLTGPSTGHPGRSPVADQPWPRRLLAAGVALLTGLSLAVAAPPAGPASAAPAFNYAEALQKSLLFYEAQQSGRLPDWNRVSWRGDSALTDGADAGLDLTGGWYDAGDHVKFGFPMAFSATMLAWGAVEYRSGYTSSGQLPHLLNNLRFVNDYFIKAHPSANVLYGQVGKGDDDHKWWGPAEVMPMARPAYKIDASCGGADLAGETAAAMAASSMVFRPTDVAYADKLLTHARQLYTFADTVRKSYHECITDATSFYRSWSGWQDELVWGAIWLYRATGDATYLAKAESEYDKLGTENQTTTRSYKWTIAWDNKQFGAYVLLANLTGKQKYVDDANRWLDYWTVGVNGQRVPYSPGGMAVLDSWGALRYAANTSFAALVYSDKTTDTTRKTRYHDFAVRQINYALGDNPRSSSYVIGFGANSPRNPHHRTAHGSWWDSQTVPVETRHTLYGALVGGPSSPNDAYSDSRSDYVMNEVATDYNAGFTSALVRLTSEYGGTPLANFPVAETPDIDELTVETTVMQAEPRATGLKAIVYNKSAFPARALTDGKFRYYFRPDGTGPVQVTAGYTQGCPSPTTAKQFSGDIWYVEVDCTGHTIAPAGQSQHRMEVQFKIGVPEGGTWDPTNDPSYQATAGPNRKVPLYSGTTRVWGDEPGPVVPDTAAPSVPGTPVASAVTSNGLTLTWAASTDTGGSGLAGYEITQSQTGSDALVLRTATTNTLAVTGLLPERTYQFTVRALDGAGNRSASSAAVSVTTPAGPAPDTTAPTAPGTPVASAVTATGLTLSWAASTDNVGVTGYRVYREAGATDALVASPTGTTAAVTGLTASTAYQFYVVAVDAAGNNSAASAPVAVTTAPPPVGGTCKVGYATNDWSTGFTATVTITNTGTTALTGWTVRWSFTGGQTVSQGWSATVSQSGTAVTATNLSYNGALAPGASVSFGFNGNHSGSNPKPTAFTVNNTACTIA

SEQ ID NO: 57 - protein, 857 aa, Catellatospora methionotrophica
MPTHSDGIRPPGRWRRLFAGTCGAVLLSSLAVATTAAPAQAAAPYNYGEALQKSLFFYEAQAAGPKPSWNRVSWRGDSALNDGSGVGVNLTGGWFDAGDHVKFGFPMAATATMLAWGAVEYRSAYAASGQLTPLLNNLRFVNDYFIKAHPSANVLYGQVGNGGTDHQWWGPAEVMKMARPAYKIDSSCGGTDLAAETAAAMAAASMVFRPTDATYANTLLSHARQLYTFADTVRKKYSECITDASSYYNSWSGFNDELVWGAIWLHRATGEAAYLAKAESYYANLGTEPQSSTHSYKWTHDWDDKTYGSYVLLAKLTGKQQYVDDANRWLDYWTVGVNGQRITYSPGGQAWLQQWGSLRYSANTAWVAFVYSDWITDATRKARYHDFAARQINYILGDNPNNRSYVNGFGTNPPKNPHHRTAHGSWTDSLTSPVNNRHVLYGALVGGPGSANDAYTDDRQNYQMNEVATDYNGGFTSALVRMYGEYGGTPLASFPPAETPDDAELYVESGLNTTGTTFTEVKALVYNKSAWPARALSNGSFRYYFTLDGSTTPSQISTSVAYSQCSPPTGPTQHSGNVYYVTVSCAGQNIIPAGQSDWRREVQFRITSSGTWDVTNDWSYQATMARNSKITLYDGTTKVWGDAPGGGTPDTTAPSTPGTPSASSITSTSATLTWTASTDTGGSGLAGYRVYREAGTTDVLVGSPATNTLALTGLTASTSYTYYVVALDGAGNTSTASTPVAFTTSGTGGGASCDVAYVIESQWPGGFSAKVTVKNTGTTTVNSWTLTYPFPATGQAVSSGHSANWSQSGATVTATNLSWNGVLSPNASTTIGYNGLWTTTNPEPTAFKLNGATCTVS

SEQ ID NO: 58 - protein, 834 aa, Nonomuraea polychrome
MRKAAFIAAVSLALPLVVAGPAAPASAAPAFAYGEALQKSLWFYEAQQSGALPDWNRVSWRGDSGMNDGKDVGVDLTGGWYDAGDHVKFGFPMAFSATMLAWGAVEYRDAYASSGQLTHLLNNLKFVNDYFIKAHPSPNVLYGQVGNGGTDHAWWGPAEAMQMNRPAYKIDASCGGSDLAGETAAAMAASSIVFRPTNPTYADTLVTHAKQLYSFADTVRKKYSDCITDAQGYYNSWSGFNDELVWGAIWLHRATNDASYLAKAESGYANLGTEPQSTTKSYKWTIAWDDKSYGAYVLLHKLTGKQQYLDDANRWLDWWTVGVNGSQVKYSPGGQAVLDRWGSLRYAANTSFAALVHSDSITDATRKARYHDFAVRQINYALGDNPRNSSYVIGFGNNPPKNPHHRTAHGSWTDQITNPEQTRHTLYGALVGGPPDPNDAYTDKRDDYVMNEVATDYNAGFTSALARLYKEYGGAPAANFPVQETPDGPEIFVEAGVNASGSNFTEIKAIVRNQSAWPARVLSDGSFRYYFTLDGSTTASQISVSSAYTQCKAPTLHSAGGSTYYVDIDCSGQVIAPAGQSQHRREVQFRISSAGTWDPANDWSYRNIPTTPGATPARTQNITLYSGATKIWGEPPGPEEEDDTPPSKPGKPAVSGITGSTAKLSWTASTDNVGVTGYDVYLGSTKVGTATSASFDLSGLTPSTSYTASVVARDAAGNTSAPSDGTPFTTTNEEPPPGGCTAAFKVTSSWGGGYQAEVTVKNTSTSAINGWTVAWTSQNQISQLWGGKHTQNGSSVSVKNEAWNGTLAPNATATFGFIAGGTASTPDPITCTPA

SEQ ID NO: 59 - protein, 870 aa, Thermobifida alba
MSVTAPPPSPGGRRGPARRLLTALGSAAALAAGFLTLPAAVGTAHAEPAFNYAEALQKSMFFYEAQRSGALPEDNRVSWRGDSGLTDGADVGLDLTGGWYDAGDHVKFGLPMAFTATMLAWGAIESPEGYTLSGQMPHLKSNLRWVNDYFIKAHPSPNVLYAQVGDGDADHKWWGPAEVMPMERPSYKVDPSCPGSDVAAETAAAMAASSIVFADDDPAYAATLVEHAEQLYAFADTYRGEYSDCVPAGSFYNSWSGYQDELVWGAYWLYKATGDASYLDKAEYEYDFLSTEQQTDIRSYRWTVAWDDKSYGAYVLLARETGEQKYLDDANRWLDYWTVGVNGQRVPYSPGGQAVLDTWGSLRYAANTAFAALVHAETVADDPVRSQRYHDFGVRQINYALGDNPRNSSYVVGFGANPPRNPHHRTAHGSWTDSIASPADNRHVLYGALVGGPGSPNDAYTDDRQDYVANEVATDYNAGFSSALAMLVAEYGGTPLADFPPEEEPDGPEMFVEAQINTPGTTFTEIKAMIRNRSAWPARMLDEGTFRYWFTLDEGVDPDGITVTSAYNQCAAPDGVHHVSGDLYYAEIDCTGEEIFPGGQSQHRREVQFRIAGGEGWDPSNDWSFQGIGDELAPAPNIVLYDAGEPVWGNAPEAGEDPGEEPGEDRTPPSAPADLAATDVTATGAVLTWRPSTDHSGSGLAGYDVYAVEGDRAVLVGSTTQTSYPLTGLRAATAYTYRVAARDNDGNVSEHSSVSFTTLAGSGGGSSCLVEYATNDWNGGFTGSLRITNTGTEPLSAWELAFAFPAGQRITQGWNAVWTQSGTDVTATSLPWNSSLPPGTTVEVGFNGSWSGSNPHPAEFTLNGESCALA

SEQ ID NO: 60 - protein, 933 aa, Microbulbifer thermotolerans
MKSTKYLMKPLCAIIAGATLAVDAIAAPNYGEALQKSIYFYEAQQSGPIPDWNRNEWRGDSGMTDGADNNVDLLGGWYDAGDHVKFGFPMASSATMLAWGVVENREAYEQTGQLQHMMNNLRFVADYFVKAHTAPNEFWGQVGNGGLDHGWWGSAEVMPMERPSYKIDANCPGSDLAGETAAALAAISMVFESTDAAYATNLLEHAKQLYSFADNYRGKYTDCITDAASYYNSWSGYNDELVWGAAWLYRATGEQSYLDKAVSYYDNLSTEPQQPIKSYRWTHAWDDKSYGSYVLMAKLTGDDQYRADAERWLDYWSTGYEGNRVTYTPGGLAHLDQWGAARYAANTAFIALIYSDYLQSADPGNSRAQDYYNFAVGQMEYLLGDNRMNMSYQIGYGSVYPTSPHHRTAHGSWNDSLQDPVENRHTLVGALVGGPDTSDNFVNDRGDYVLNEVATDYNAGFTGALARLWLDFGGNPIPDSQFPAPETRDVELFVDAKLNSSSARHTEISSYVHNRTAWPARLTDNLSLRYWIDISELVDAGYGPEDLVLSSGYNQGSGFTGPHPWGDPADNIYYVEVSFAGVDIYPGGQSASKKETQYRIGAPEGAPWDPENDPSWDGYNSTRKEAPLIALYDDGELVWGQEPGGGCGGDTGVNCAPSADSQNLSTPYETSLSVTLSGSDSDGSIAAYTVVNGPANGTLEGSGASRLYTPAAGFFGTDSFTFTVTDNDGATSQPATISIEVEAPEIPAVNITSPVDGARFAPGADVRVQYNLQNAEGVNLYLDGTLVATGSGSGTLTVQAPDEIGFYMLKLVATQDGEELSASDSVTFEVAEAPAGSLGCTVGQMDDWGAGFVLNNITVTNEGSEPVDGWTVLLDFGQEVTGNHWNASLSSTSGSQLSASNVSYNGSLQPGQSATFGMQGTPGNLGAVTCTAQ

SEQ ID NO: 61 - protein, 880 aa, Lentzea flava
MLNRRSRAITTAIVVLFAGLVVPRAEAAPAFNYAEALQKSIWFYDAQRSGKLPADNRVSWRADSALRDGSDVGLDLSGGFYDAGDHVKFGLPFAFSMSMLAWGAVENRAAYADSGQLKHLMANLRWGTDWIIKAHPSPNVVYGQVGAGNPDHAWWGSAEVMPMARPSYKVDASCPGSDLAGEYAAAMASASMVFKDTDPTYAATLLTHAKQLYTFADTYRGKYSSCITDAANFYNSWSGYQDELVWGAIWLHRATGDASYLTKAKAEYQKLSTEPQSSERSYKWTIAWDDKSYGAYVLMAKLTGEQTYITDANRWLDFWTTGYNGNRVRYSPGGQAVLDSWGSLRYAANTAFAALVYSDWLSTRDSARARTYHDFAVRQVNYALGDNPRNSSYLVGFGANPPRNPHHRTAHGSWADSIQEPVTSRHVLYGALVGGPSTNNDAYTDSRSDYVMNEVALDYNAGFTGALARLYREFGGTPLASFPVAETPDGPELTVQASVNQSAAQFTEIKAFVLNKSAWPARTFNGTLRYFFTLDGATTPGQISVTTNYNQCGTASAPTQYRGAVYYVEIPCSGIAPAGQSAYRKEVQFRITSTGTWDPANDWSYAGLGSGNTVAQTDRIVLRQDGKTVWGTEPGGGVDDNEAPTVPTGLTASNAGSSSVTLNWTAATDNVGVTGYEVMNGTSVVGSATTTSLLVTGLVPDTQYTFSVRARDAAGNVSAASAPVSVRTLPGVRSTLSVQHKGTSAARTNQIGATLQVTNNGTGPVSLTTTTVRYWFTGEPAAGYQVWCDWAQIGCAGIRAQVVKLATPRPGADAYLEVSFTTGSLAANAATGEIQLRVAKSDWSAFDQADDHSYRVSAALTDFDRVTAYSAGALAWGAEP

SEQ ID NO: 62 - protein, 969 aa, Kibdelosporangium banguiense
MVTTVLVALLAGLIGPLPSVASAAPAFNYGEALQKSIWFYDAQRSGKLPADNRVSWRGDSALRDGSDVGRDLSGGFYDAGDHVKFGLPFAFSMSMLAWGAVENRDAYAGSGQLSHLMANLRWGTDWIIKAHPSANVVYGQVGAGDPDHAWWGSAEVMAMARPSYKVDTSCPGSDLAGEYAAAMASASMVFKESDPAYAGTLLTHAKQLFTFADTYRGEYSDCITDAAKFYNSWSGYQDELVWGAIWLYRATGDAAYLTKAKAEYLKLSTEPQSPERSYKWTIVWDDKSYGAYVLMAKLTGEQTYISDANRWLDFWTTGYNGNRIRYSPGGQAVLDTWGSLRYAANTAFAALVYSDWLSTRDSARAKTYHDFGARQVNYALGDNPRNSSYLIGFGANPPRNPHHRTAHGSWADNIQEPAVSRHILYGALVGGPKSNDDAYTDSRSDYVMNEVALDYNAGFTSALARLYKEYGGTPLANFPVAETPDSTELTVQASVNQSNTQFTEIKAFVLNKSAWPARIFNGTLRYYFTLDDATTPSQLAINTNYNQCGTPSAPTQYSGSVYYVEIPCTGVSPAGQSAYRKEVQFRITSSGTWNPANDWSYAGLGTGNTVVQTDGIVLRQDGKVVWGNEPTGTIDREAPSVPTALTASAVSGSSVTLSWAASTDNVGVTGYDVLRGGTVIASTTGTTYQATGLTPNTSYSFAVRAKDGAGNVSAASSAINVRTADTPIDTEPPTAPTGLRASSIGSTSVSLSWTASTDNVGVSGYEVLNSTTVVGSPTTTSLQVTGLTPDTSYTFSVRAKDSSGNVSAAVPITVRTTPATASTLTVQHKGTGSASSNQIGATLQATNRGTTSVGLTTVTLRYWFTGDASSPSYQVWCDWAQMGCANIRAQVVKLGTARTGADAYLEVSFTSGSLAPGASTGEIQLRVAKTNWSTFNQADDHSYRTSNSLTDFDRVTAYSGGTLSWGVEP

SEQ ID NO: 63 - protein, 1065 aa, Piscirickettsia litoralis
MVNLIIAFWGAAEVMQMARPAYRINTENPGSDLAAETAAALASMYLVLKRAGGDYAVYAEQKELLRHAKELFDFANQYRGKYSDSIRDAAQFYRSWSGYEDELVWAALWLYKATGEAHYLTYAENNYNNLLDEDHNGIQGRTAWTHAWDDKSYGSYVLLATLTGKEQYQHHAENWLNRWACTQTTDCVRRTSGGLVWLDQWGSLRYSANTALIAFIYADYLAKNNIKEDLVTSYQRFAKRQINYMLGDNSRQSSYVVGFGNNPPKNPHHRTAHGTWLGGSPLSIPNPSRHTLYGALVGGPGSTDDMDYIDNRNDYIRNEVALDYNAGFSGALARMVMEFGGRPLDNFPENAVRDDEFALEAKVNAQGVNFIEISALLRNKTAWPARITDNLSFRYFINLKEEINAGLTPENIAITTAFNQGAQVSALQVFDKEKAIYFIEIDFSGEAIAPGTQESHRREVQFRLSIPAELGISMNYQNDWSFREVIDGELTQDGRHLTHYIPVYEGNIRVFGLAPDSAPVNQAPIIDEDKSITIDQPSVPTALAISAPIDPEGGEVEVVITKVPVGASVIANSTGQVVHSGQKIAVNELTQLTVSYLGSNLTNDEFSYQAVDSAGNISTQRIRLLVENEAPIFGPNKTIAIYNQQYDQEIALNLPQPTDSNHDALTITVSRLPEHGQLTALGMVIRQGQTLSIAELSALNISYPIGTPINLVETLELIAQDARGGSAILTVQLTTKEQDLQQCTVKTELNEWENGFVAKLTVYNNSNLVVNDWQLDFKTKNYQVSNIWNADIATQGGVSSVSGKPYNQAIQVNNFISFGFQGVGHFQVPQAYLLNGQPCQQIGLSYKNRAPLMGENKIIQAAINDIVPLHLTLPEDPEGGVLDLNLVSVSEGVTVFAAGKKVFSGQSLTLSEFSQLAVQFPANTLANSSANLSLSAKDKQGARTVQTIRLIAKASDDFLVNCEVDFKIKDQWQNGFVADITIKNIGSELINGWQLTWDYNSQLINIWNARGEMKAGRVTLKNELYNSQIPAQGTVSFGFQGQGAAEVPEGILLNEEPCRIQSSNS

SEQ ID NO: 64 - protein, 790 aa, Myceligenerans indicum
MHPRPPARRRAAVRATALTAAWALLTAGAAGVAPAASAAGDQDYAEVLQKSLFFYDAQRSGDLPDDFRVNWRGDSGMADGADVGLDLTGGWYDAGDHVKFGLPMAFTTTMLAWGAIESPGGYAAAGQQDELLDSLRWVNDYFIRAHPEPDVLYAQVGDGEADHKWWGPAEAMQMARPAYKVDASCPGSDVAGETAAAMAASSIVFADSDPGYAAELVTHAEQLYDFADTYRGEYSDCVPAANYYNSWSGYQDELVWGAYWLYEATGDASYLAKAEAEYDQLGTEPQTGTHSYRWTIAWDDKSFGTYALLAQATGEQRYIEDANRWLDYWTTGYDGERVTYSPGGQAYLDTWGSLRYAANTAFVALVYGDWLAQQGDTTRARTYHDFGERQIDYALGDNPRGSSYVIGYGDNPPQNPHHRTAHGSWLDSLTDPADTRHTLYGALVGGPGSPDDSYTDDRSDYVSNEVATDYNAGFTSAVAYLADEYGGTPLSDFPQPEQPDQDEFFVESNLNQPGTEFTEIKAMVRNRSAFPARVLDDVRVRYWFTLDAGADPATIQASTNYTECPDGAATVGHAGGDRYYVEIDCSGSPVYPGGQSQHRREIQFRVTADVWDPSNDWSQAGVGADPAKNDRITLYEGPELLWGSEPGSVGPEPEPTEEPTVEPTGEPTGDPTEEPSGQPTDPGPGTASCTASYTVVNQWDGGFQGEVSVTNTGTETVEGAAVTWEFTNGEAITQSWGGTAVQDGPTVTVQAPGWAPDLAPGATWTLGFIATGSSVPPAAVLLGDTPCDLG

SEQ ID NO: 65 - protein, 803 aa, Microbispora amethystogenes
MRRTFPALAALIISSAVVPAAVPSTAAAAPAYNYAEALQKSLFFYEAQQSGKLPSTNRVSWRGDSALNDGKDVGLDLTGGWYDAGDHVKFGLPMAFSTTMLAWGAVDYRDAYTSSGQISYLLNNLRFVNDYFIKAHPSANVLYGQVGNGGDDHKWWGPAEVLPMARPAYKIDASCGGSDLAGETAAAMAASSMVFRPTDAAYADKLLTHAKQLYTFADTVRGKYSDCITDAAGYYKSWSGYNDELVWGAIWLYRATGDSSYLAKAESAYSGLGTEPQSTTKSYKWTIAWDDKSYGCYALLAKLTGKQQYADDANRWLDWWTVGVNGQKVAYSPGGEAVLDQWGSLRYAANTAFVALDYSDWLTDATRKARYHDFAVRQINYALGDNPRKSSYMVGFGANPPRNPHHRTAHGSWSDNLNEPAQSRHILYGALVGGPTSANDAYTDSRSDYTMNEVATDYNAGFTSALVRLVKEYGGTPLAGFPGTETPDGPEIYLQASVNATGSTFTEIKALVVNHSAWPARALSDGSFRYYFTLDGDTTPGQITLSSAYTQCKQPTGPTQFSGKIYYVTIDCSGQVVAPQGQSQSRREVQFRIASSGTWDPSNDWSYKGVATTPGSTPVTVENITLYSGSNKIWGNAPGTEQSPSPSPSASPSDSPSPSPSPSVSPSPPRSPSPSPTPTCPPQNTGSCDPPLSPSPSATPTQQGKACTATYKITSSWQGGFQADVTVKSSGTAAITGWTVGWTLPSGQSISQLWSGTLSQSGSSVTVKNLSWNGSLAPGATTSFGFTGNGNGSVPSPVTCTPA

SEQ ID NO: 66 - protein, 1058 aa, Cellulomonas persica
MRHRATSPHPSGALRRRGRTRVAAGLSLTLAASALVVPLATSASAAPDFNYAEALQKSMFFYQAQASGDLPDNYPVSWRGDSGLQDGSDVGVDLTGGWYDAGDHVKFGFPMAFTTTMLAWGAVESPTGYTKAGQLDELKGNLRWVNDYFVKAHTAPNELYVQVGGGEADHKWWGPAEVMTMARPAYKITASCPGSDVAGETAAAMAASSLVFKADDPTYAAKLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQKIAYSPGGMAVLDSWGALRYAANTSFVALVYSDWTTDSTRKARYHDFGVKQINYALGDNPRKSSYVVGFGANPPTMPHHRTAHGSWLDSITTPAASRHVLYGALVGGPSAANDAYKDDRQDYVANEVATDYNAGFTSALAYLVDQYGGTALASFPTPEQPDGDQMFVEAQLNQPAGGTFTEIKAMIRNQSAFPASSLKNAKVRYWFRTDGFAASDVTLSSNYSECGAQSGKGVSAGGTLGYVELSCVGQNIHPGGQSQHRRELQFRLTGPSGWDATNDPSFAGLTTTGLAKAPAITLYDGSTLVYGKEPTGTTPGDTTAPTVPGTPVASSVTTSGATLTWAASSDDTGVVAYDVYRVQGTTSTLVASPTTVTTTLTGLSAGTAYTYNVKARDAAGNVSAASSSVTFTTTEIPADTTAPTVPGTPVASSVTSTGATLTWTASTDAGSGLAAYDVYRVQGTTSTLVASPTTTTTTLTGLTPATAYTYVVRARDAAGNVSAASSAVTFSTLEIPADTTAPTVPGTPTASGITTTGATLTWAASTDAGSGVAKYEVLRVSGSTQTLVASPTTTSVALTGLTADTSYSYVVRAVDAAGNTSAASSAVTFRTLGSTPTQTCTVVYTPNSWNTGFTASLKITNTGTSALTWALAFDLTAGQKVSNGWSATWTQTGTSVTATGLSWNQTLAPGASTEIGFQGTHSGSNPSPTSFTVNGSVCG

SEQ ID NO: 67 - protein, 1046 aa, Cellulomonas gelida
MRRRRGRTRVAAGLSLALAATAIVVPLATSASAAPAYNYAEALQKSMFFYQAQASGDLPAGYPVSWRGDSGLTDGSDVGVDLTGGWYDAGDHVKFGFPMAFTTTMLAWGAVASPDGYTKAGQLDELKGNLRWVNDYFVKAHTAPNELYVQVGDGEADHKWWGPAEVMAMARPAAKITASCPGSDVAGETAAAMAASSIVFKGDDPTYAAKLVSHAKQLYSFADTYRGAYSDCVTGASAYYKSWSGYQDELVWGAYWLYKATGDATYLAKAEAEYDKLGTENQSTTKSYKWTVAWDNKQFATYALLAIETGKQKYVDDANRWLDYWTVGYNGQKITYSPGGQAVLDSWGSLRYAANTSFVALVYSDWVKDATLKARYHDFGVRQINYALGDNPRKSSYVVGFGANPPTKPHHRTAHGSWLDSITSPAQSRHVLYGALVGGPGSANDAYTDDRQDYVANEVATDYNAGFSSALAYLVDEYGGTPLASFPTAEQPDGDQFFVEAQLNQPPGGTFTEIKAIVRNQSAFPARSLKNAKVRYWFRTDGFAASDVTLSSNYSECGAQSGKGVSAGGTLGYVELSCVGQNIYPGGQSQHRRELQFRLTGPSGWDATNDPSFVGLTSTGLAKAPAITLYDGSTLLFGTEPTGTTPGDTTAPSVPGTPVASSITTSGATLTWAASTDDTAVVAYDVYRVQGTTSTLVASPTTTSTTLTGLTAGTAYTYNVKARDAAGNTSAASSSVTFTTTAVPTDTTAPSVPGTPVASAVTSTSATLTWTASTDAGSGVAGYDVYRVQGTTSTLVASPTTNSTSFVGLTPGSSYTYNVKARDVAGNVSAASASVTFSTVEIPPDTTAPTVPGTPVASGITKSGATLTWAASTDSGSGLAKYEVLRVSGTTLTLVASPTTASTTLTGLTADTAYSYVVRAVDQAGNTSAASSAVSFRTLADTPTGTCSVVYTPNSWNTGFTASLKITNTGTSALTWALAFDLTAGQKLSNGWSATWAQTGTTVTATGQSWNQTLAPGASAEIGFQGTHSGSNPSPTVFTINGTACA

SEQ ID NO: 68 - protein, 804 aa, Cellulomonas avistercoris
MPRTSARRARSRAVWAGAAVVAVVAGAVTLPAQAAQAAPAYNYAEALQKSMFFYQAQRSGDLPADYPVSWRGDSGMDDGKDVGKDLTGGWYDAGDHVKFGLPMAFTATMLAWGAIESPAGYTQAGQADELRDNLRAVNDYFVKAHTAPNELYVQIGKGDDDHKWWGPAEVMTMARPSYKISASCPGSDAAAETAAAMASASIVFKSSDAAYSATLLTHAKQLYSFADTYRGKYSDCVTDAQSFYKSWSGYQDELVWGAYWLYKATGDATYLTKAESEYDKLSNENQTTTKSYKWTVAWDDKSYATYALLAMETGKQKYVDDANRWLDYWTVGVNGAKVTYSPGGMAVLDSWGALRYAANTSFVALVYSDWLTDTTRKARYHDFGVRQINYALGDNPRKASYVVGFGANPPKNPHHRTAHGSWLDSLKDPVETRHVLYGALVGGPGSANDAYTDDRGDYVANEVATDYNAGFTSALAYLAGEYGGTPLAGFPTAERPDQDQFFVEAKLNQPQSGAFTEVKAIIRNRSAFPARSLTNASIRYWFTLDEGVPASSLSVSTNYTECGSQPAAIKHASGSLYYAEIGCAGQNIHPGGQSQHRREIQFRVQGTLQWDATNDPSFTGLPATGDPVKTKGITLHEGSTLLWGTVPGTVDPTPTPTVTPTPTATPTPTVTPTPTATPTPTPTPTPTPTPTPTPTPTSGPATCAVTYSANSWSTGFTASVRLKNTGSTPLTWSLTFDLTAGQQLQQGWSATWAQTGTKVTATGAAWNTTLVPGASVDLGFNGSHTGQNPAPTSFAVNGTTCAAG

SEQ ID NO: 69 - protein, 1051 aa, Cellulomonas xylanilytica
MPRPRSRGRVAIACTTAFTVAAGLLVAPLAGAATAADPTYNYAEALQKSMFFYQAQRAGDLPADNPVSWRGDSALTDGADVGKDLTGGWYDAGDHVKFGLPMAFSSTMLAWGAIAAPQGYTKAGQLDELKGSLRWVNDYFVKAHTAPNELYVQIGDGEADHKWWGPAEVMTMARPSYKVSAACPGSDVAGETAASLASASIVFKTDDPAYSATLLSHAKQLYSFADTYRGKYSDCVPEAAAYYKSWSGYQDELVWGAYWLYKATGDATYLAKAEAEYTKLSVENQTTTRSYKWTVAWDDKTYAMYPLLAMETGKQQYVDDANRWLDYWTVGVNGQRIPYSPGGQAVLDSWGSLRYAANTSFVALVYSDWLTDPTRKARYHDFGVRQINYALGDNPRKSSYVVGFGANPPQNPHHRTAHGSWLDSLTQPEKTRHVLYGALVGGPSGPNDTYTDLRSDYTANEVATDYNAGFSSALARLTQEYGGTPLAGFPVAEQPDGDEIFVEAMLNQPAGGTFTEVKAMIRNQSAYPARALKNGTLRYWFTLDSGDSAANLTLSANYSECGPQTAKPVSAGGTLYYAELSCVGQNIHPGGQSQHRRELQFRVTGGSTWNAANDPSFAGLGTALAKTKAITLYDGGKLIWGTEPTGTVTDTTAPSVPGTPVAAGITSTGASLSWTASTDAGSGVAGYDVFRVQGSTSTLVASTTTASTVLSGLTPSTAYSYTIKAKDVAGNVSAASSAGTFTTADPPVEVVVPGRPGTPVASAITSSGATLTWSASTAGTYPVAGYEVHRVVGTTATLVASTTGTSTALTGLTPGTAYTYTVRAKDSSGNVSPSSVGVTFTTTAATDTTAPTVPGTPSASSVTQTGATLTWAASADTGGSGLAGYEVYRVQGGTSTLVASPTTASTMLTGLAANTAYTYAVRAKDGAGNTSAASPSVTFTTLPTTSTSSCAVTYSANSWNVGFTGSIKVTNTSTSPLTWSLAFSFPSGQKLTQGWSATWAQTGSAVTATGLGWNATLAPGASTDIGFNGSHTGTNTAPTAFTVNGATCTVG

SEQ ID NO: 70 - protein, 924 aa, Streptomonospora litoralis
MPHPPSPYASSPPTPPPRRRSAARRRVTAAAACAALAAGLLASPTATGTADAAPDFNYAEALQKSMFFYEAQRSGELPEDNRVSWRGDSALQDGSDVGLDLTGGWYDAGDHVKFGLPMAFTTTMLAWGAVENPGGYTSSGQMRYLKDNLRWVNDYFIKAHPEPNVLYAQVGDGDADHKWWGPAEVMPMERPAYKVDPSCPGTEVAGETAAAMAASSLVFAEDDPDYAATLVRHAEQLYDFADTHRGDYSDCVPAGSFYDSHSGYQDEIVWGAYWLYRATGDESYLDKAETEYDNLSTEPQTDTRSYRWTIAWDDKSYGVYALLAMETGKQRYIDDANRWLDYWTVGVNGDRVPTSPGGQAVLDTWGSLRYAANTAFVALAYSDWLDQGTRKQRYHDFGVRQIDYALGDNPRDSSYVVGFGADPPVNPHHRTAHGSWLDDITAPEDNRHVLYGALVGGPDSPDDAYTDDRQDYVANEVATDYNAGFSSALAALVADYGGTPLADFPVPEEPEGPEIYVEAALNQPGTTFTEIKAMIRNKSGYPARMLDEGTFRYWFTLDEGVDPAAIEVTSAYNQCADPGGARQAEGDLYYVEIDCTGQEIYPGGQSEWRREVQFRIAGGEHWDPSNDWSYQGVGEELARAENIVLYDAGERVWGTAPDGGGDDGDDGDGDGTPDTEAPSAPGGLTADAATAGSVSLSWQPASDDTGVAEYTVHASADGSGEPVATVDGAQATVTGLEPDTEYTFTVRARDAAGNTSPSSAPVTAATSAEPEAGPLAALYRASGSDPSDSQIRAHLAVQNTGDAPVDLGRVSLRYWFTRDGGSAAELESHCDWAQLGCGGIARTIGDAPAVPGADTYLELSFDGGTLEPGGSTGPIQIRLNRSNWQAFDESDDHSYDGSAASFTENPNVTVYVDGEPVWGAEPAA

SEQ ID NO: 71 - protein, 920 aa, Nonomuraea spiralis
MIAAASLVLPLLAGVGPAAHAAAPAFAYGEALQKSLWFYEAQQSGDLPGWNRVSWRGDSGMNDGKDAGLDLTGGWYDAGDHVKFGFPMAFSATMLAWGGVEYRDAYAGSGQLTHLLNNLKFVNDYFIKAHPSANVLYGQVGNGGTDHAWWGPAEAMQMNRPAYKIDASCGGSDLAGETAAAMAASSIVFRPTNPTYADTLVTHAKQLYSFADTVRKKYSDCVSDAQGYYNSWSGYNDELVWGAIWLYRATNDAAYLAKAEAGYDNLGNEPQTTTKSYKWTIAWDDKSYGAYVLLHKLTGKQKYLDDANRWLDWWTVGVNGSKVKYSPGGEAVLDRWGSLRYAANTSFVALVHSDSITDAERKARYHDFAVRQIDYALGDNPRNSSYLIGFGANPPKNPHHRTAHGSWTDQITNPVNSRHTLYGALVGGPPDPDDVYTDKRDDYVMNEVATDYNAGFTSALARLYKEYGGAPAASFPPAETPDGPEIFVEAGVNASGTNFTEIKAIVRNQSAWPARVLSDGSFRYYFTLDGSTTAAQISVSSAYTQCKAPTLAPAGGSVYYVNIDCSGQAIAPAGQSQHRREVQFRISSAGSWDPSDDWSYKGIPATPGATPVLTKNITLYSGATKIWGEPPGPEEQDTTAPSKPGKPAVSGITGTAAKLSWTASTDNVGVTGYDVYLGSTKAGTATGTSFDLSGLTPATAYAAHVVARDAAGNTSAASDATTFTTTGTEDRTLPSKPGKPSVTGITGSGAKVSWAASTDDVGVTGYDVYLGSTKVGTATGTSYDLGGLSSATAYTVSVVARDAAGNSSAASDGTSFTTADAPAGGCAATFKVTNAWGGAYQAEVTVKNTGAAPVTGWTAAWSSAGQITQLWGGKLTQTGSAVSVRNESWNGGLAAGATTTFGFIANGTPATPDPITCTPA

SEQ ID NO: 72 - protein, 895 aa, Albitalea terrae
MTIRHGSRLRTPTSRGPRWTSWLTALATSAALLACGGGDPASTTTGRADASHTAPASAAATPAHNYAEALQKSVLFYEAQQSGKLPSWNRVSWRGDSRLSDGADVGHDLTGGWYDAGDHVKFGFPMAFTATQLAWGALRFEAGYKAAGQYDALLNNLRFVTDYFMRAHTAPNELYVQVGQGSTDHSWWGPVEVYPLAAPSFKISASCGGSDVAAETAASLASASMVFKASDAAYATQLLTHAKQLYTFADTVRAKYSDCVTDAANYYKSWSGYNDELAWAAAWLHRATGDSAYLAKAESFVNTSSSGFGTEGQSSFLPYKWTHDWDSKHYGVYVLLAETTGKAVYKDAIERNLDYWTTGTSTGERVSYTPGGLAWLSQWGSLRYAMNEAFIALAYADVVSDEAKKQRYRDFAVRQVNYVLGDNPRRGSYMVGFGSGSPQHPHHRTSHGSWMDSQTTPAQHRHVLYGAMVGGPGSDDSYADEIGNYVTNEVATDYNASLTGVLAKMNELFPGNVPLANFPQREQPSEDEIFVEAGINSSGSNYTEIKALLNNRSGWPARHGDKLSLRYYVDLGEVIAAGHSASDVTLSSNYSQGGTVSGLVRCGTSNLYYAVLDFTGTDIYPGGQSAFRHEIQFRFSVPAGTSYWNPANDPSYAGLSAAGTVKSLAIPVYSNGERVFGQEPAACGGVSVGVPTAPEGVTATGGSGSIALTWGAVANATGYRILRSATGAAGTYAQVGVAASPRFTDSGLAAATTYHYVVIATNAQGDGPPSADVSATTSTGGGGNGDCTLTLDASNDWGAGQVLVVKLANSGTQPLSNWQISWTESSDFTVQNSWSGSVSTSGRTVTVKPASWNSSIAPAGAIELGMQISYSGSRPLPTNAAVQGRSCTVSVISSR

SEQ ID NO: 73 - protein, 983 aa, Roseateles terrae
MSTLIQTRRGRTRWAAPCLALTLAACGGGDQTRSTASADAGPSAKALATSSGVKAAAAPAYNYAEALQKSILFYEAQQSGKKPEWNRVSWRGDSRLTDGSDVGLDLSGGWYDAGDHVKFGFPAASTATQLAWGALEFEAGHKDAGQYDALLRNLRWINDYFIKAHPSANVLYGQVGNGSTDHSWWGPVEVYPKEAPAYKIDATCGGSDLAGETAAAMAAASMVFKTSDPSYSATLLTHARQLYDLADTKRQKYVECITDAQGYYNSWSGYFDELAWGAIWLYRATGEASYLTKAESFVTTGAGGFGSEGQTAYLPYKWTQDWDSKHYGSYLLLARLTGKQKYKDAIERNLAYWTTGTSEGERVTYTPGGLAWLSQWGSLRYALNESFIAMVYAKDVSDSTKQQRYRDFAMQQLNYALGSNPRNSSYLIGFGANSPQHPHHRTAHGSWADSQTLPANHRHVLYGALVGGPGANDAYTDSISDYVSNEVAIDYNASIVGVLAGAQQLFPGSVPLANFPQAEVPTDDEYFVEAALNSAGTTYTEVKAVVNNRSGWPARVTDKLSLRYYVDLSEVFAAGRTAADIKVSANYSDGGVARGLVRCGSSNIFYAVGDFTGTKVYPGGQSAYKKEIQIRLAAPDSTTFWNPSNDPSYAGLGTTGSAPLKVSGITLYDNGRKVWGNEPTACGGVAVPVPATPSGLTATAGIGSISLSWAATANATGYTLKRSTTGTAGSFALLASPTGTSYVDSGLAGSTRYYYTVSARNDSGESAVSDAVSATTPAGPPAAPAPTAAAGDGKVTLSWSAVTGAASYEVSRASSATGSYTSLASGLSALSYVDSGLTNGSAYFYKVGARNSAGVTVSDPVSATPVGSGGGGGGSSNCTLTLDTSNDWGSGQVFRLLLSNTGTTPISNWGVSFTESNSITVSNSWSGTFVATGTKVSFTPQTWNATVAAGGNTDAGMQLSYSGTKPTPSAVVMTGASCQVVIK

SEQ ID NO: 74 - protein, 946 aa, Candidatus Lokiarchaeota archaeon
MKDFRILFTKKVKFLLILFIFLFQSTLFLGNSINKNMINNHELKINNWEEIPITDTNGEALYNYATMVSRTILFYDANQCGNNITGNNRFNWRGDCHLNDGIEWTLNNGETIWIDATGGYHDAGDHIKFGITNAYAASTLGWGLYEFRDQYEETGQLDDILRVIKWATDYFIKCHPEPNSFLYSVGSGEDHNYWGPPELQLDSRSPRDIIVCTPSNPASDVCGLTAGALALMYINYYDIDEEYANLCLKHAKEIFELGRNNLGLSDGLAFYPPSIYWDDLAWGALWLYAASNDISYYNEFLTILGPSCAGIDHDDDIENGLNWENQWTHCWDAVWAGVFAAAAGITAHPAFLEQVKINLDFWLYDLDETPAGLKYLNEWGVLRYSTASALCGLVYYKYTNDIVYRDYAASQINYALGSNPLQRCYIVGCGNNPPQHPHHDAAHGSETGFLDDPLEHKHILYGALVGGPGMDDQHNDATDDYVQNEVSIDYNAAMVGALAGMRKYFGPNLIPDPDPLPEEPIQAYWVESKIEKDQNERSQLTFYVQNHAIHPPHYENNLIFRYFVDLSEVFEQDYGLENVNIEVVVNENQEAQVSQELIPWDELNYIYYVEISYQGVELYNKREVQIALIYNTPNFEGIWDPTNDFSHLPMTDQLESNENIPLYRGEELIWGLEPYKDRIPPEPPSGIITTVKGSAQIDLDWNDNTEEDLSYYNIYHSTAVNFECNMDSFIGSSKTSEYSDTELLQETTYNYKITAVDSNQNEGMPSEIVSATTLTPDPYPPDPPTGLSIISTSSSQIELDWNDNTESDLSKYKIYRSTSMGFICDQVNFIGDTLVSIYLDSNLLANTIYYYKIIAIDTSNNPSNPSFEIIAQTDKILAGLKVQYRSGNLNQLSSEIRYELILFNNGPLDVALTDVTMRYWFTSEPSLSDLTYSCDYAAVGASGIST

SEQ ID NO: 75 - protein, 885 aa, Halomicrobium salinisoli
MTRETRDRSQTGESETNGGGSPHRTERRTFLKAGLVGLGTVAAGGPGAGVASAVAKDDYNLAHALQQSLYFFDANECGDGVTGQPLEWRGDCHLVDESVPLRQSIDEGGSNVPESYVDEHSDVLDPNGDGTVDLSGGFHDAGDHVKFGLPQAYAASTLGWALYEFEDAFREIGSDDHIRRHLRRFTDYFLKSIFRDDDGTVVAFSYQVANGNFDHEYWGPPELQDNEEMPRPAYLATPETPASDQCGAAAAALALQSLNLEDEDPEYAERCLDNAQALYEFGVEHRGLGHGGEFYPSSSDADDLAWAAVWLHVATGEQRYLEDVIGTDGSGNYTGHVGEIIATTEDEWNNTWVHSWDTVWSGVILKLAGITDDQQWWDLARWHLEYWTGGEVSHDGSSGNYIQTTPAGFSYLDQWGSARYNASAQFLATVYRKQGGEKGEALTDWARGQMQYIMGDNPFGYSLIVGFTEDHAEEPHHDAAHGSLVGQPSDPPKPKHTLWGALVGGPDDQDEHNDETDDYIYNEVAIDYNAGFVGALAGLYTYYGDGDEPVEGFPPDEEPIDPYYVEAQVEGNSAETVEIKAFINNVAVHPPRFEDGLSFRYFFDASGIDDPEGLTVDVFYDQVGSQLGQPAEVVGPEPYDESEGVYYVEFDYSGTAIAGRYELTFALSDYTNASLDASTDWSFQDLGDGLTETEYMPVYVDGELAFGREPGGNAPDTTAPGAPSPVSVTSTGTTSLGLSWSAVTDDGSGLDQYVVAVDGTERTTVPAGTTSATVDDLDPDTDYEISVRAVDGAGNESEPATATATTDTEGGGDGPEPLDGTRPTDPDGDGVYEDLNGNGETDYSDVVRYFDEIDGEYFAENAEYFDFNGNGEADYSDLVALFQEV

SEQ ID NO: 76 - DNA, 3135 bp, Cellulomonas palmilytica
  source 1..3135: mol_type = genomic DNA
gtggtttcctggtcccgccgccgtgcacgcacggctgtcctgtctgccggcgtcaccgccgcagccctgatcaccgcgacggtcgtcgcgcccgccgccctcgccgaccccgtggagtcgtcgcacgacttcgagtccgggacgcagggctggtacagctacgggggcgactcgtcgtcctcggtcgtcgacggcgagctctgcgtggtcgtgcccgccggcacgacgaacccgtgggacgtcgcggcgcagcacgacgacgtgacgttcgaggcgggcggcacctacacggtgtcgttcaccgcgcacgcgaccgaggaggtctcggtcaacctgcgggcgggcatcgggtggccggacgacgtgtcgtcgaccgtcgccctggcacccgaggagcaggagtactcgttctcctggacccccgagttcagcgggtccggcaacctgtcgttccagctcggggcgcaggacgtcgactacacgttctgcctcgacgacttccagctggtcggcgaccgggagctgatcccggacaccagcttcgacggtgtgctgcccgagggctggagcgcggccggctggaccgtgacgtccgagccgggtgagggcgagccgctgtgcttcgaggtcccggccgactcgaaccagtacgcgggtctcgtgctcaacggcctgccgatcgaggagggctccaactaccgcctcacgttcacggccagctcgagcaacgccgcgacgatccgcacgatcgtcggcgagaacgccgagccgtggcgcgcggccttcgccgacaacacgccgctgtcgaccgagctgaccgagcacgtgctcgcgttcacctcgcccgtgtctttcccggccgagtccgacgacccggcggtcggcgtcgggcaggtcgcgctgcagatgggtgaccgcggcgcgttcacgttctgcatcacgagcctgtcgctcaagaaggtcgccacgccgccgccgccgtacgagcccgacgtgcgcggtcccgtgcgcgtcaaccagctcggctacctgccggacgggccgaagaacgcgacggtcgtgagcgacgcggccgagccgctcgcgtggcggctcgaggactcgtcgggcaccacggtcgcttccggtgaggcgacgcccgccgggctcgacgcctcgtcgcagctcgaggtgcagacgatcgacttctcgtcgtacaagaagtccggcaccggctacacgctcgtcgtgggggaggaccgcagcgacccgttcgcgatcgcgtcggacctgtacgcccagctgcgctacgacgcgctgaactacttctacccggtgcgctcgggcatcgcggtcgacgtgccggacgaccggtacgaccggcccgcgggccacgtgtccgggccggacggcgaggtcaacaagggcgatctcgacgtgaagtgcctcaccgcggccgaggacggcgcgagctggtcctacggcacgtggacctgcccgacgggctactcgctcgacgtcgtcggcggctggtacgacgccggtgaccacggcaagtacgtcgtcaacggcggcatcgcggtcgcgcagctcctgagcacgtacgagcggaccctgcacacgaagaacgcggcgaagggcgccctggcggacggcacgctcgacgtgccggccgacgagagcggcaacggcgtgccggacgtcctcgacgaggcgcgctgggagctcgagttcttcctcgcgatgcaggtgcccgcgaagtccggcatgacggtctccgcgaccgaccagcgctcgctcgacgggctcgtgcaccacaagatccacgacgtcggctggaccggcctggggctcctgccctcggaggacccgcagcagcgacgcctgcaccggccctcgacggctgcgacgctcaacgtggccgccacggccgcgcagggtgcgcgcctgttcgagaagtacgacaaggcgttcgcccagcagctcctgagcgtcgcgcggtccacgtacgcggccgccaagcgggtgcccgacctgtacgcgccgcagtccgcgggccagaacggcggcggaccgtacgacgacacgaacgtgaccgacgagttctactgggcggccgccgagctctacctcacgacgggcgagaaggccttcgagaaggacctgcagaacagccccgtcgcgacgagcgacatctgggagcgttccgggttctcgtgggggagcgtggcggcgctcggtcgcctcgacctcgcgacggtgccgagcaagctggcgacccgcaaggccgtgcaggcgtccgtcgtggccggggccgacaagctcctcgcgtggcagcgggccgaggcgttcggcaccgcgtacccgggcaacgacgacggctcgtacgtctggggctcgagctcgtccgtgctcaacaaccaggtcgtcctcgccacggcgtacgacctgacgggcgacgcgaagtacgcgaacgcggtgctggagtcgatggactacctgctgggtcgcaacgcgctgaacaactcgtacatcacggggtacggcacgaagttctcatcgcaccagcacagccggtggctcgtcccgccgccgccggggacgatctcgggtggcccgaactcgcagagcagcacgtgggacccggtcatgaaccggatctacccgcccggtcacgactgcgccccgcagctctgctacatcgacgacatcgaggcgtggtccgtcaacgagctgaccatcaactggaacgccccgatgtcctgggtcgcctccttcgtggccgacctcggcgtgagcggggtcccggccgcgccggtcgtgaccaagcacccgtcgtcggtgtcggcgaagaagggcacctcggtcgcgctgagcgctgctgcggccggcaagcctgcgccgaccgtgcagtggcaggtgcgcaacgcgaagggcgcatgggtcgacgtgcccggcgggaagacgacgacgctgcgggtcatcgtgtcggcggcgaccgacggcgcccggtaccgcgcggtgttccgcaacgcgtccggcacggtcgtcacggacgtcgcgacggtcacggtgctgcccggccccaagggccccgcgccgaagggcccgggtccgatcgtccccgcacccaagccgggtcccggccgcgggtga

SEQ ID NO: 77 - DNA, 3639 bp, Cellulomonas gilvus
  source 1..3639: mol_type = genomic DNA
gtggtttcctcgaagtacccctggcgtgcgccccggtccgggcacgcgcgcactgcttcgctcgtcggtggcgtggccgccggcgctctcgtgatcggcgtcgccgtcgcccccctggcggccgccgaccccctcgacgaggtgcatgacttctccgacggcacccagggctggtacggctacgggggaggacccgccatgtcgaccggcgtcgtcgacggcgagctgtgcgtggtcgtccccgccgggaccgacgagccgtgggacgtcgcgatccagcacgacgacatcgacttcgccgcgggtgaccggtacacggtcgggttcaccgcgcacgcgacgtccccggtcacggtcaacctgcgcggtggcatcggctaccccgacgacgtggcctcgtcggtctcggtcggcaccgagaccgacgcctactccttcacgtgggagcccgagttctccgggagcgggaacatctcgttccagctcggcgcgcaggcccaggactacacgctctgcatcgacgacttcgtgatcgacagcggccaggagctcgtgcccgacacgacgttcgacggcgtgctgcccgacggctggaccaacgacggctggacggtcacgtcggaggccggcgagggcgagccactgtgcttcgaggtccccgggaccgccggcacgtacgcggggctggtgctcaacggcctgccgatcgaggagggcggcaactacgagctcacgtacaccgcgagcgcgtccaacggcgcgacgatccgcacggtcgtcggcgagaacgcggcaccgtggcggaccgcgttcgtcgacaacaccgagctcagcacggacctgaccgagcacgcgctcgcgttcacgtccacgttcacgttccccgcggagtcggccgaccccgcggtcggcgtgggacaggtcgcgctccagatgggcgcgcgcggcgacttcacgttctgcatcacgagcctgtcgctcaagaaggtcgcgaccccgccgccgccgtacgagcccgacacgcgcggtccggtgcgcgtcaaccagctcggctacctcacgaacgggcccaagaacgcgacggtcgtgagcgagtcggccacgccgctcgcgtggcagctgcaggacgcctcgcacgccgtcgtcgccgagggtgaggcgacgcccgcgggggtggacccgacgtcgcagctcacggtgcagacgatcgacttcagcgacgtcaccgcggcgggccagggttacaccctggtggtcggcgaggaccgcagcgacccgttcgcgatcggcgacgacctgtacgagcagttccgctacgacgcgctcaactactactacccggtgcgctcgggcatcgcggtggacgtccccgacgaccgctacgaccggcccgcgggccacgtggacgggcccgacggtgccgtcaacaagggcgaccaggacgtcgcgtgccttacggcggccgacgacggtgcgtcgtggtcctacgggtcgtggacgtgcccgcaggggtactcgctcgacgtcgtcggtggctggtacgacgcgggcgaccacggcaagtacgtggtgaacggcggcatctcggtcgcgcagctcatgagcgcgtacgagcgcacgctgcacgtggaccacgcgagcgacgacgcgttcgcggacggcacgctcgacgtgccggccgacgagagcggcaacggcgtgccggacgtcctggacgaggcgcgctgggagctcgagttcttcctggcgatgcaggtgcctgcgggctccggcatgacggtctcggacaccgacgaccggtcgctggacggcctggtgcaccacaagatccacgacgtgggctggacgggcctggacctgctgccgtccgccgacccgcagcagcgccgcctgcaccgcccgtcgaccgcggccacgctcaacctggcggccacggccgcgcagggcgcgcgcctgttccgtgcgtacgacgccgcgttcgcggacgagctgctcgaggccgccgagaccgcgtacgcggccgcgcagcgcgtgcccgacctgtacgcaccggcctcggccggcgccaacggcggtggcccgtacgacgacgccgacgtgtccgacgagttctactgggccgcggccgagctgtacctgaccacggccgccgacgagtacgaggcggacgtgctggcgagcgagcacgccgacgacgacatctggacgcgcggtgcgttcagctggggcgcggtcgccgcgctcggccggatggacctggccacggtgccgagcgagctgccgaccagggccgcggtgcgtgcctcggtggtcgagggcgcgcagaagtacctcgcgtggcagcaggccgaggccttcggcacggcctacccgggcggcgaggacctctcctacgagtggggctccaactcgatggtgctcaacacgcaggtgatcctggcgaccgcgtacgacctgacgggtgagccggcgttcgcggcggccgtcgtcgagtcgatggactacctgctcgggcgcaacgcgctcaacaactcctacgtgacgggctacggcacgacgttctcgagccaccagcacagccggtggctcgtgccgccgatgccgggcacggtcgcaggtggaccgaactccaagcgcggcacgtgggatcctgtgatgaacgggctctaccccgagggccacgagtgcgccccgcagctgtgctacgtcgacagcatcgaggcgtggtcggtcaacgagctcacgatcaactggaacgcgccgctggcgtgggtcgtctcgttcgtcgacgacctgggtgcgggcgtcacggcccaggcgcccgtggtgaccacgcagcccgcgtccgtgacggtcgcgctgggggccaaggccacgttcacggccgcggcgagcgggacccccgcacccacggtgacgtggcagtggcgggcgcccggcggcacgtggaagacggtcgcgggtgcgacgagcccgtcgctgacggtcacggcgaccgccgccaccgacgggcgtcagtaccgcgcggtgttcaccagctcggccggcaccgccacgagcgacgtcgccacgctgcgcgtccgtgcggtcaagcccgtggtcaccacgcacccggcgtcggcatccgcggcgctggggcgcaaggtcacgctcagggccgacgcgtccggctacccgacgccgacggtgcgctggcagcagcagcggcccggctcgtcgacgtggacggacgtgaagggcgcgacctcgcgcacgctcgtcgtcgcggtcacgcgggcgaccgacggcgtccggtaccgcgcggtgttcaccaaccgcgcgggctcggcgacgtcgcgtgccgcgaaggtcacgctggtgcgcgccgctccgcggttcgtcgaccaccccgacggcacgaccgtgcgtgccggtcagaaggtgacgctctcggcgaccgtgctggcctacccggccgcgacgctcacgtggtacgtcaaggcgcccgggagctcgagctggaaggccgtgcccggtgcgacgggcacgcggctcacgctcgtcgcgagccggtcgctcgacggcgcgcagtacaaggtggtcgcgcgcaacgcgctcgggtccgcgtggtcgaaggccgcgctgctccgggtccgctga

SEQ ID NO: 78 - protein, 1054 aa, Cellulomonas sp. PSBB021
MAAPCGHLARLRGDDVVSWSSRRARTAVLSAGLTAAALVAATVVAPAALADPVGSTHDFESGAQGWHSYGGASTSSVVDGEFCVVVPTGTKDPWTVAAQHDGITFEAGTTYTVSFTAHATQDVSVNLRAGIGWPDDVASTVALTSQEQEYSFTWNPEFSGEGNLSFQLGAQAADYTFCLDDFELAGDAELIPDTSFDGVLPEGWSAAGWTVTSEPGEGEPLCFEVPAGSGQYDGLVLNGLPIEEDANYRLTFTASASNAATIRTIVGENAVPWRAAFADNTQLSTELTEHELAFTSPVTFPAQSDDPAVGVGQVALQMGARGDFTFCITSLSLKKVATPPPPYEPDVRGPVRVNQLGYLPDGPKNATVVSDATEPLEWRLEDSSGATVASGDATPAGLDPSSQLTVQTIDFSSFRTAGTGYTLVVGEDRSDPFAIAKDLYAQLRYDALNYFYPVRSGIAVDVPDDRYDRPAGHVSGPDGEVNKGDLDVACLTAAEDGAAWSYGTWTCPTGYSLDVVGGWYDAGDHGKYVVNGGIAVAQLLSMYERSLHSPDAARGALKDGTLDVPADESGNGVPDVLDEARWELEFFLQMQVPTGSGMTVSATDKRSLDGLVHHKIHDVGWTGLGLLPSQDPQERRLHRPSTAATLNVAATAAQGARLFAKYDKAFSQQLLSVARSTYAAAKRVPDLYAPQSAGQNGGGPYDDTDVSDEFYWAAAELYLTTGEKTFETAVQSSPLATSDIWARSGFSWGSVAALGRMDLATVPSKLPTRKAVQASVVTGAEKILAWQQAEAFGTTYPGDADGSYVWGSSSSVLNNQVVLATAYDLTGQARFANAVLESMDYLLGRNALNISYITGYGTKFSSHQHSRWLVPPPPGTIAGGPNSQSSTWDPVMNRLYPPGHDCAPQLCYVDDIEAWSVNELTINWNAPMSWVASFVADLGVSGVPTAPVVTKHPSDVSAKKGTSVALRATASGKPAPSVQWQVRDAKGRWVDVPGARTTTLRIVVTPATDGAQYRAVFRNASGTATTDVARVTVLAGPKGPGPKGPGPIPWPGRG

SEQ ID NO: 79 - protein, 1044 aa, Cellulomonas palmilytica
MVSWSRRRARTAVLSAGVTAAALITATVVAPAALADPVESSHDFESGTQGWYSYGGDSSSSVVDGELCVVVPAGTTNPWDVAAQHDDVTFEAGGTYTVSFTAHATEEVSVNLRAGIGWPDDVSSTVALAPEEQEYSFSWTPEFSGSGNLSFQLGAQDVDYTFCLDDFQLVGDRELIPDTSFDGVLPEGWSAAGWTVTSEPGEGEPLCFEVPADSNQYAGLVLNGLPIEEGSNYRLTFTASSSNAATIRTIVGENAEPWRAAFADNTPLSTELTEHVLAFTSPVSFPAESDDPAVGVGQVALQMGDRGAFTFCITSLSLKKVATPPPPYEPDVRGPVRVNQLGYLPDGPKNATVVSDAAEPLAWRLEDSSGTTVASGEATPAGLDASSQLEVQTIDFSSYKKSGTGYTLVVGEDRSDPFAIASDLYAQLRYDALNYFYPVRSGIAVDVPDDRYDRPAGHVSGPDGEVNKGDLDVKCLTAAEDGASWSYGTWTCPTGYSLDVVGGWYDAGDHGKYVVNGGIAVAQLLSTYERTLHTKNAAKGALADGTLDVPADESGNGVPDVLDEARWELEFFLAMQVPAKSGMTVSATDQRSLDGLVHHKIHDVGWTGLGLLPSEDPQQRRLHRPSTAATLNVAATAAQGARLFEKYDKAFAQQLLSVARSTYAAAKRVPDLYAPQSAGQNGGGPYDDTNVTDEFYWAAAELYLTTGEKAFEKDLQNSPVATSDIWERSGFSWGSVAALGRLDLATVPSKLATRKAVQASVVAGADKLLAWQRAEAFGTAYPGNDDGSYVWGSSSSVLNNQVVLATAYDLTGDAKYANAVLESMDYLLGRNALNNSYITGYGTKFSSHQHSRWLVPPPPGTISGGPNSQSSTWDPVMNRIYPPGHDCAPQLCYIDDIEAWSVNELTINWNAPMSWVASFVADLGVSGVPAAPVVTKHPSSVSAKKGTSVALSAAAAGKPAPTVQWQVRNAKGAWVDVPGGKTTTLRVIVSAATDGARYRAVFRNASGTVVTDVATVTVLPGPKGPAPKGPGPIVPAPKPGPGRG

SEQ ID NO: 80 - protein, 1152 aa, Sanguibacter inulinus
MVSPRHARQALAVLTASSISAALVLATVVAPAATASPLSTEHTFTGGDPEGWHSYANSGSVTTSTATEELCSTVSGGTNPWDVAAQHDDLTFDRDTTYTMSFDARADQAVTLPLQVGATYPATSARQVVLDGTATPQHVELTVSPATWPTTSTDAGSPLADGWTTTSGLAVLQLGAQAVPYTVCIDDVSITERTDPTDPTDPTDPTDPTDPTDPTDPAEQVVGGDFEDGAIAPFYAVGPGVTSAVTDGVLCVELGAGTSNRWDQIVGYNGIVIEPGTTYTLSFDASSSNGRPVRAIIGDDEDPYTVLLENNPTLSAGMRSYTYTFTAERGFPTDGATSPGIGEISFQVGGSDTPWTFCLDDVSLLSGTETPPYTPETGPRVRVNQVGYLPAGPKEATLVTDAPAALPWELHDAQGVTVASGTTVPAGLDPTAGVAVHEIDFTGVTAPGTYTLVADGEESYAFTVGADIYQQLRADAINYFYPARSGIEIDGSLMVGQPDAQKYSRPAGHVGTPDGADGGPNRGDVDVACLTPETEGQYWMYGDWTCDYTADVTGGWYDAGDHGKYVVNGGIAVAQLLSTYERTLTSPTGAGADLRDGTLNIPLDEQTNGVPDVLDEARWELEWMMKMQVPADAAMYPGMVHHKVADADWTGLPLLPSQDAQERYLHRPSTAATLNFAAVAAQGARLWEQYDPQFAADLLAAGRVAWDAALRTPDLYAPAPNDDPSTGSGPYDDDSVGDELYWAAAELFLTTGDQVFEDHVLASEYHTADIWTSGGFNWFETAALGRLDLATVESDLPGRAAVRDSVVEAAERYLAWQQGEPFGTAYPGSDGIYSWGSNSAILNNQVVLGTAFDLTSDQRFADAVLESMDYLLGRNALNNSYVTGYGQQFSQNQHSRWFAHSLEPSLPNPPRGSVSGGPNSDAVTWDPVIKALYGPDRMCAPQACYVDDIQSWSTNEITVNWNSALTWVASFVADQQDGDASGAGTVVVVTSDPADVTVVDGQQASMSAAATGSPAPGVQWQRLTAGVWTDVPEATSTTLTLTARLGEGTQYRAYFTNAFGGVYTEPATLEVTESPEVVAPSPAPVVDPPVEETARGAGGGAPDGRGGALATTGADPAWVVVAGLLLVAGFTVTGVARRGRRSRDGSAPGARA

SEQ ID NO: 81 - protein, 1089 aa, Cellulomonas xiejunii
MRAATAITSAGVLAAVGLLPAVAVDDGFDDGPGAWIAYGTDGGIDTSSGALCVDVPADSAQYGVGVVLNGVAVEEGATYTFAYTASATTDVTIRALVGQNGAPYGTVLDTTPALTSEATSVEETFTASASYPATPTGESPEGQIAFQLGGFTDEAWTFCLDDVALSSDSELLPHTSFAEGLGPWDLYGTSEPTFADGEMCVEVPGGNANPWDAGLSFTGLPIEEGQNYVLSLTGRTEPATPVRVIVGEGGGAYRMVLEQSAAPLGVEQTTLSYPFTAGLAFPADGEAPGQVAIHLGKSAAYTFCLSEVSLTTSATPPPPYEPDTGPRVRVNQVGYLPFGPKRATLVTEATDAVAWQLLDADGDEVATGESQPVGADASSGENVHVIDFSDVETAGAGLTLVADGETSHPFDIAEGLYSQLRYDALNYFYLARSGIDIEASIVGEEYAREAGHVGVAPNQGDTAVPCIGPRDYYDGWTCDYTLDVTGGWYDAGDHGKYVVNGGIAVAQLLSTYERTLTTAGARAGALDDGTLNLPEHGNGVPDVLDEARWELEWMLKMVAPSGEYAGMVHHKIHDEGWTGLPLAPADDPQARSLHRPSTAATLNVAAVAAQGARLFAEYDEQFAATLLETARSTYEAALAHPDVYAPGAAGSDGGGPYDDSDVTDEFYWAAAELYVTTGEDRYATDLTASAHHTGDVFGPGGFHWGGTAALGRLTLATVPNDLPDRADVRASVVAGADRYLAAQAEQPWGSVYSPTGGSYDWGSNSSVTNILVVVATAYDLTGDEKYLRGTLEGLDYLFGRNALNQSYVTGWGTVSSQNQHSRWFAAQLDPSLPNPPPGSLAGGPNSMRGTWDPTMQATLPEDCAPATCYLDEISSWASNEITINWNSSLSWVASFAADQEDGGVVAVAPAITTQPVDVTVALGGTATFTAAASGVPAPQVQWQVREGGKWRDVPGATAATLQVVATAAKAGTQYRAVFTNASGSATTDVARLLVEKQAPVVVKHPADVSGRIGTFVSFTAGASGNPTPTVRWQVRWGSGPWLPVPFAHSTTLRVLVTPLAYGTQYRAVFTNPAGTAATDGAHLTPRFGR

SEQ ID NO: 82 - protein, 1121 aa, Cellulomonas cellasea
MVSFPDASARASGAAAATVAVLALAAAVSPVALAGPLTDVHDFATGTEGWFSYPPEAVTASAETGELCAAVGAHTGNPWDVAIQHDAVTFDRDATYTVEFDAHASRAVTVPVQGGPGYPAVFGQSVQLDGSSTPEHVELTFSPSAWPTGEGSPVADDWSTATGNVSFQLGNQGEPYELCVDDFSIRPLGANLIPNGDFASGSTDPWFVAGDLGPADVSSGALVVPVPAGSGIYAGVGFNGLAIEEGTSYTLTFDVSASEERTIRAIVGENGGSYGTVLDKTVPVTTEPTTVSHTFTATDSYPATTTPGGPFEGQLAFQVGGRGGDWTFTLDNVSLVESLTPPPPYAPETGPRVRVNQVGYLPAGPKQATLVTERTEPVVWQVRTVSGGVLAEGVTTPRGVEPSSGLNVHTIDFGELATEGEGLILAADGETSRPFDIDADLYQQLRYDALNFFYLMRSGTEIDAAIDPDYARPAGHVDVAPNTGDGAVTCLTAADEGAGWAYGDWTCPEGYALDVRGGWYDAGDHGKYVVNGGIAAAQLLSTYERTLHAGTADPGALADGTLAVPESSNGVPDVLDEARWELEFLLSMQVPADGGEYAGMTHHKIHDVGWTGLPLLPSQDPQERRLARPSTAATLNLAAAAAQGARLFAEHDAEFAAELLAAARTAWDAAQAHPAVLAPDAAGNNGGGAYGDGTVTDEFYWAAAELFLTTGENAFAEHLLASPHATGDVFGPGGMNWGSTAGLGRLDLATVPNALPTRDAIRQSVLDAADAYVATQDAQAWGSTYVRPDGLYEWGSNSMVLNTTVVIATAFDLTGDETYRRSALEAVDYLLGRNALNISYVSGYGEVSAQHQHNRWFPLSVPGVSPEGSLAGGPNSTTGTWDPTMQAAFAGGCAPSMCYLDELGSWASNEVAINWNSALSWVASFVADQGDGSADASTPVRVTGQPADVTVAPGATATFTAAATGVPAPTVRWQVQRPGKRWVDVPGATSTTLTVTATLADHGTLYRAVFTNASGSETTDVARLAVVAAEPLTVRLSAPAVRAGGHVTVAVRGATAREIVQVRLGDELLETRAVRKDGTVSISVHVPRHTDPGAAVVTVRGSSSGREGTATLQVLPAPRRS

SEQ ID NO: 83 - protein, 958 aa, Amycolatopsis bullii
MRVRRFRAGLVLLAVTTLGLTAGSVPASAADYERLLNGTFASGTLDPWWAGAGTTGRVTGGEFCTDVTGGTANGYDALAGQNGVPFEAGQQYTLTFDAHATTTQQISAVAGEAVSPYRQISRTDLTVTPTAQHFTVTFTSTLDFPAAGNGQLAFWFGGQAANNTVCLDNISLIGGVIPPGGLPPTSGIRVNQESYTPGLPKHATLISSSATPVTWTLRNAAGTAVTTGQTRPRGADAASGESVHDIDFSAYDTAGTGYTLAVGSETSFPFDISADGLKKLRYDSLAFFYHQRSGIAIDAQYVGANYARPAGHVNVAPNQGDNNVPCRSDLNCGYTLDVRGGWYDAGDHGKYVVNGGIATWELLDEYERALKLGDASALGDGKLAIPERNNGVPDILDEARWEVDFLLSMQVPDGKPNAGMVHHKIHDAAWTALPTRPELDSQPRRLSPPSTAATLNMAAVAAQASRLWKTIDPAYSAKLLAAAEKAYAAAKANPNVLADPNDGTGGGTYSDNTVTDEFYWAAAELFATTGKSGYRTDVTGSSLYRGVSFNTHGFDWGSTGALGDITLALVPTDLPAADVAAIKTAITTTADSHLAQMAGMGYPAPYRTADGTYEWGSNGLVANNGVVLALAYDFTKQDKYRDGAFGAMDYLLGRNPANYSYVAGHGDQAVQNVHHRFWAHELDPSLPTAPPGALSGGPNSGLQDPTAARLLAGCAPQRCFVDDIQAYSVNEVAINWNSALAWLANWTAEKSPSAVDTTAPAAAGKPAVSGVSATGATVTWTASSDAESGIKNYDVVSVTGAARKVLATVTGTSATLTGLSPSTAYTLVVVARNGAGLSGPDSASTQFTTLPDTPAGGCAVTYSATSWSGGFTAYVTLTNTGTTAWPDWTLKFAFPAGQKVTQGWSATWSQSGAEVTAKAMSWNASVAPGKSVSIGFNGSHTGSNPAPSSFAVNGKTCS

SEQ ID NO: 84 - protein, 882 aa, Streptomyces fradiae
MRHLPHTPPPARRRSRLVGTAVMGALIAGLVAAPSGAAEPPDARAAEGPEQIVNGDFSAGTAPWWWTANSPAAVTDGELCADIPAGTSDPWDAIIGQDGVDLLAGEGYELSFTAHASVPVAIRTSVQMAVEPYTSEHLSTDRITTEPQTFTHTFTAKADNDAAQLGFQAGGHDRAYTLCLDDVSLTGGTEAPPHQPDTGSPVRVNQVGYLTEGPKGGTFVTEATQPLPWTLNAPDGSPAATGTTKVFGQDPTSRQHVHTFDFSRVTRTGEGYTVTIAGKTSEPFAIGDDLYARLRTDALAYFYHNRSGIEIDAGLVGEKYARPAGHLGKAPNQGDGDVPCQPGVCDYRLDVRGGWYDAGDHGKYVVNGGISVAQVMATYERTLTAGNADGAPLGDGRLRVPERGNGTPDILDEAAWQLDFLMKMQVPAGKTLAGMVHHKIHDAEWTGLPLLPHQDPMKRELHPPSTAATLNLAATGAQCARLFRPYDAAFADRCLTAATTAWRAALAHPAVYARADDNVGGGAYEDADVTDEFYWAAAELFVTTGQDDYRRHLLDSPLHGDAGKVFPRGGVWWGGTAGLGALSLATVDNGLTDAQLAQVRTMVTTGADRYAEDARTAAYGVPYAPEDNHYVWGSNSGVLNNMVVLGVAHDLTGNTKYRDALLGGLDYLLGRNPLNQSYVTGYGERDSRNQHHRHWAHQLDPSLPNPAPGSVAGGPNADIQDPVAQDKLKGCAPAMCYIDDIGSWATNEITINWNAPLAWVASFVDDLGGSGTTDPDPAACRVGYASDRWNGGFTSRVTVTNTGTEAISPWKLTWAFADDQRVTDHWGSRITQTGREVTAHPASWNGTIPPGGSVAFGFNGTSGGGAADPTAFALNGAPCTGG

SEQ ID NO: 85 - protein, 803 aa, Flavimobilis rhizosphaerae
MEDTTVDTVVNAPPTAVRRPLPALLALALAVSLTACDHSSPAPPPTVPATAQTSAPANTGTELLPATSFADGLGPWALYGTDAAPRTADGATCVDLPGGTTNPWDVGLTYNGLGIDASAAYHLALTASAAPATTVRVVVGEATGAYRTATELFAPLTPTAQPTEHDFTAAIALSATDGDVGQVAIQLGSATPFTFCISEASVTTRDSMPTAASDAPAVRVNQVGYLTHAAKAATVVTDAADPLPWTLDLGGMTVARGTTEPRGTDPSAGLAVHTVDLSAVTTPGTGYTLTVDGASSDTFAIGDDLYRAMRVDALSFFYPQRSGVAIDGAVAGDAYARPAGHADVGPDQGDGAVACLPAGALTVDGKDLYDGWTCEGTIDVTGGWYDAGDHGKYVVNGGIAVGQLMSTYERALHAPTGDAGALGDGTLRVPEHGDGVPDVLSEARWELEWMLTMQVPAGKDLAGMVHHKVHDDSWTGIPLLPSDDDRTRYVHRPSTAATLNLAAVAAQGARLFEPFDAAFAARLLKAARTAWAAAAAHPDLYAPNTNLHPSPGGGPYDDTELDDERYWAAVELFLTTGEDAFADALTTNPYHVGGTSEAFGPAAFDWGHVAAYARTQLATVPNALPVLAEVRRSVVDAAEATLARQATQPFGTAYVPDGGRYDWGSNGLLLNNLAVLVAAYDLGGEDRFRQGVLTGLDHLLGRNAMGISYVTGHGTRFAQNQHSRWYAAQADPSLPHPPAGTVSGGPNSDVPDPVSSSLAGCAPQRCYVDDIGAWGVNELTINWNSALAYVVSFAADQGAAGPA

SEQ ID NO: 86 - protein, 915 aa, Lachnospiraceae bacterium
MKTRSTIRRAVALLFLALFCLSRPWPALTVLAGEQLGETDFDDGKGLPWHIVESEPAKMDFELSDGQYIITIINPGGASQGGEDRWDCQFRHRGLTIVSGHQYRVRYELTASNSGKYYTKIGNLAGDTEIWHNMSNGYDLDATWDPIPVNAGETKRVDLTFTAAQGMEVAEWAFHLGGDGQYTPGGCFPAGTVIRFDNMSLEDLSSDENDYVEEEPWARALILTNQLGYIEDAAKKATLLNDSDKPVSFSLIDENGRVAYEGKSIPYGYDADSGDMVHVLDFSEADAPGEYCIEAEDGARSRSFSIGVSSVYSSLLYDSLNYFYQNRSGIEIESSLICSGDGSELSRAAGHTPDLASVDQCWGYSASSGTQDVSGGWYDAGDHGKYVVNGGISLWLLQNMYEAALYCGTEAAYADGTMSIPENGNSFPDLLDEARWEMEWMFTMLVPDGSCRDMVYHKVHDIKWTGLGVAPADDPQERILKPPTTAATLNVAACAAQASRLWAGIDKAFAEECLDVAVRCYDAACDHPDMYAPLDESVGGGAYGDDDVSDEFYWAATELWLTTGEENYHKDMKSSPWYLAVPETMDGGESVGACGSFDWGHTAALASMSICLNEDACDSSEYKKTKDNLLETAEHYLSLCDEQGYGQPYAASSIAYNDPDNGYIWGSNSVVADNAVILAYAYLISGDSAYFDGAVSGMDYLLGRNPMDYSYVTGYGSHAVTYPHHRFWAKQIDDSFPMAPAGVLSGGPNSGMQDPWVRGMGWKKGTIPPAKCYLDHIEAWSVNECTINWNAPLAWLSGFLTEYSPGGITPGAGSFGSGVSLQTSSEERESYTAEKTDRGSVTASGKKSSRDRENDRDSEREEGSESNVLLIVIVIAASVLLLAVAVMIFVYKMAKLKALSGADPKNKGPDQPLGA

SEQ ID NO: 87 - protein, 700 aa, Streptomyces sp. MBT62
MKRRRATLLAATALLAAALTALPASPAGADEVEQVRNGTFDTTTTPWWTTSNVTAGLSDGQLCADVPGGTTNRWDAAVGQNDITLVKGESYKFAFTASGSPAGHVARAIVGLSVSPYDTYYEISPELSETGNSLAYTFTSPVDTTQGQVAFQLGGSADGWHFCMDDVSLLGGVPPEVYEPDTGPRVRVNQVAYLPGGPKNATLVTASTAVLPWELKSAAGAVVAHGRTVPRGTDVASGQNVQSIDFGAYRGRGEGYTLVADGETSRPFDIGTAAYERLRLDSLKYYYTQRSGIAIRDDLRPGYARPAGHLDVAPNQGDANVPCQPGLCDYTLDVTGGWYDAGDHGKYVINGGISTWELLSTYERSLLARTGQPGKLRDGTLAIPESGNKVPDVLDEARWELEFLLKMQVPDGQPLAGMAHDKVHDEQWTGLPLLPSQDPQKRELHAPTTQATLNLAATAAQAARLYRPYDREFAAKALKVARKAWAAALAHPAVYPDPNDGTGGGAYPDDNATDEFYWAAAELYLTTGERQFADHVVNSPLNTADIFGSLGFDWARTAALGRLDLATVPSRLPGRDKLRRSVIEGADTYLATLRSEPYGMPYAPPGNLYDWGSNSQILNNAHVIATAYDLTGAAKYRDGALQSMDYILGRNALNMSYITGYGEANSHNQHSRWYAHELDPTLPNPPDGTLAGGPNSSIKD

SEQ ID NO: 88 - protein, 795 aa, Kineococcus sp. TRM81007
MHPARKRRARALTAATALLAPVSLLLTPTAAHAADQIVNGAFDQGTTGWTIYPNPSVQDGAGCATVPASTAAYGAAIQQEIPLVAGESYKLTFRARTTPATEGPVRVVVQGGADVNHQQFLPADKPAFASAFGTHEYYFTAERDYPNAQLAFQQDVVNPVGYTLCVDDVVLEGGAERPPYVPETGPRVRVNQETYLPDGPKGATLVTDATTSLPWELKDAAGTTVAKGTTTPRGVDPTAGLNVHTIDFSRYAEGGTGYALVADGETSHPFDIGEAAWERLRDDAMTFFYTNRSGTPISDELTPGYGREAGHVGVAPNQGDTAVPCQSLDDDSQEILVAQGDEPWTCDYTTDVTGGWYDAGDHGKYVVNGGIAVAQLLQQYERSLHAPTADRGALADGTLRIPEAGNGVPDVLDEARWELEWFLKMQVQPGRPLEGMVFHKVADVDWTGLPLDPAADPQPRVLYRPSTAATLNVAATAAQGARLFAEFDPAFSERLLAAARTAYAAARANPELYAPAPDATLDPNPGSGPYDDDDVSDEFYWAAAELYLTTGEPQFRKDVLRSPVHTADVFPAGGFDWGHVGALGRLDLATVPSRLGKHELRQVRRSVTEAADRLLAAQATQPFGQAYAPEDGQYAWGSNAQILSNLQVLGTAFDLTGKDAYRDGVVRSTDYLLGRNALNNSYITGYGDVFSRNQHSRMYANQLDPASPNPPAGTVAGGPYSPTATSGDPITAALFPDGCVAQFCYVDDIGSWSTNEITINWNAPMAWVASFVADQDSGCDAGRGHGRGHGGGHRG

SEQ ID NO: 89 - protein, 1117 aa, Cellulomonas persica
MVSWFPRRARTAALSAGLTATALVATAAAAPLAFADPVESPHDFTDGTQGWYSYGGDSSSSIVDGEFCVVVPAGTTNPWDAAAQHDGVTFAAGDTYTVSFSAHATVPVSVNLRAGIGWPDDVSSTVALTEDEQDYAFTWTPGFSGDGNTSFQLGAQAQDYTFCLDDFQISGEVELIPDTSFDGVLPEGWSANGWNVTSEPGEGEPLCFEVLDGSGQYAGLVLNGLPIEADANYRLTYTASSSNAATIRTVIGENAEPWRTAFVDNTQLTTDLQEHVLAFTSPVTFPAESSDPAVGVGQVALQMGARGDFTFCITSLSLQKVATPPPPYEPDVRGPIRVNQLGYLPGGPKNATVVSDASEPLDWQLQDAAQQVVASGQATPAGLDPSSQLDVQTIDFSAFQTPGTGYTLVVGEDRSDPFAISATLYDQLRYDALNYFYPVRSGIAVDVPDDRYDRPAGHVSGPDGAVNKGDLDVACLTAAEDGASWSYGTWTCPQGYSLDVVGGWYDAGDHGKYVVNGGISVAQLLGTYERTLHRKNVVAGALGDGTLDVPADESGNKVPDVLDEARWQLEFLLAMQVPAGSGMTVSATDTRSLDGLVHHKIHDVGWTGLGLLPSDDPQQRRLHRPSTAATLNLAAAAAQGSRLFEKYDKAFAKELLKAAKTAYAAAERVPDLYAPVTAGQNGGGPYDDTDVSDEFYWAAAELFLTTGDPAFRKDVLASPDHTGDIWSRTGFSWGSVAPLARMDLATVPSKLPKKDVDAVRASVVAGADTYLAWQEAEAFGHAYPGQADGSYEWGSNSAVLNNQVVLGTAYDLTGDPRYARAVLESMDYLLGRNALNNSYITGYGTQYSSHQHSRWLVPPLPGTISGGPNSQRGTWDPVMNRLYPAGHECAPQMCYVDDIEAWSVNELTINWNAPMSWVASFVADVAAKGLPAAPVVTTQPQDATVAPGATASFVVAATGSPTPTVRWEQRDAKGRWKPVHGATSTTLTVKASPATDGAQFRAVFTNPSGSATTDAATLRITRQAPTITVHPASASGKQGTKVTLRAAATGYPTPRIQWQVLEKGSTSWVDVRRACGTSLQFVIGPPTDGDRYRAVFTNTAGSATTREATITLARGPR

SEQ ID NO: 90 - protein, 1176 aa, Cellulomonas fulva
MAAPLASAAPIDQVHDFSDGPQGWYSYPDTAPSSVVDGEYCVVVPGGSANPWDIAIQHDGLTFEAGTDLTASFRAHATADVSVNLRAGTGWPDDVASTVAVTTVAQDFAFSWEPGFSGDGNVSFQLGGKSADYTLCLDDFSISGAQELVPDTSFDGVLPEGWTNEGWTVTSEPGDGEPLCFEVPGTSGTYSGLVLNGLPIEEGGNYELRYTASASNGATIRTVVGENAAPWRTAFVDSTELSTELTEHVLPFTSGVTFPAQSSDPAVGVGQVALQMGDRGDFTFCITSLSLRKVATPPPAYEPDTRGPVRVNQVGYLTNGPKSATVVSDATDPLAWQLQDADEHVVAEGTATPAGTDPTSQLAVQTIDFREFTTPGEGYTLVVGDDRSDPFAIGADLYDQLRYDALNYYYPVRSGIAVDVPDDTYDRPAGHVDGPDGAVNKGDVDVACLTAADDGASWSYGSWTCPEGYARDVVGGWYDAGDHGKYVVNGGISVAQLMSLYERSLHTDHATDGALADGTLDVPADESGNGVPDVLDESRWELEFFLAMQVPQGSGMTVSADDDRSLDGLVHHKIHDVGWTGLGLLPSADPQQRRLHRPSTAATLNVAATAAQGARLFREYDADFADRLLEAAQTAYAAAQRVPDLYAPASAGSNGGGPYDDTDVSDEMYWAAAELYLTTGDDEYEADVLASEHSDDDIWSRSGFSWGSVAALGRLDLATVPSDLPTRDEVRASVVEGAEKYLGWQEAEAFGTAYPGSDDGQYEWGSSSSVLNNQVVLATAYDLTDDPRFSDAVAESMDYLLGRNALNNSYVTGYGTQYSTHQHSRWLVPPMPGTVSGGPNSRRGTWDPVMNTLYPAGHECAPQACYVDDIEAWSVNELTINWNAPLSWVASFLADLGEGVAPQAPAFTQQPHGVTVALGATATFTVQVTGTPTPTVAWQWRTGAGTWQTIPRATGTTLSVVASAATDGRQYRAVLTSTAGTATSSVATLVVKAQRPVVTKHPTSASAAVGRSVTLRASASGYPTPTVRWQLRKPGSSSWKNVAGGTRTWLTVRLSTANTGTQYRAVFTNRGGSATSRAATVRLAQAAPRFTSHPASTTVRAGQKATFTVTVAAYPKATLTWYVRTKGSASWKAVTGARWSTLSVTATRALDGAQYRAVARSSAGSAWSKVATLTVR

SEQ ID NO: 91 - protein, 1103 aa, Cellulomonas sp. PhB150
MVSFLPGRRRATWAGAATAGLATLALVVAPMAAANPIGEVHDFADGPQGWFSYGGTSSSSVDDGELCVVVPAGTTNPWDVAAQHDDVTLAADSPVTVSFRAHATADVSVNLRAGIGYPDDVSSSVALTSAPTDYSFTWTPGFSGTGNTSFQLGGQASDYTFCLDDFSISGDDELLGTTTFAGDTLPAGWSLPADWAVTSAAGDPAFCVAVPASAGQYDGLVYNGVPVEDGGNYALSFTASASNGATVRALVGENGGAYRTAYVANPALTAELASYDYPFTSSLAFPADEGAVGQVALQLGARGDYTFCVSEVSLRKTATPPPAYEPDTRSRVRVNQVGYLPQGPKRATVVTDAAAPVAWELHAAGGALVASGKAALAGTDPTSGLKVQTVDFSTVTAKGAGFTLVADGETSDPFAIKTDIYAQLRYDALNYFYPARSGIAIDAPGTVYDRPAGHVSGPDGAVNKGDLDVACLTAADDGASWSYGTWTCPAGYARDVVGGWYDAGDHGKYVVNGGIAVQQLLSTYERTLYATDAAKGALKDGTLEGPADESGNGVPDVLDEARWELEFFLAMQVPAGSGMTVSSTDSRSLDGMVHHKIHDVGWTGLGLLPSDDPQQRRLHRPSTAATLNVAATAAQGARLFEKYDKAFAAKLLAASRTAYTAALRVPDLYAPASAGANGGGPYDDTEVSDEFYWAAAELYLTTKSSTYKKDVEKSAHRTDDIWTEGGFNWGSTAALGRLDLATVPSDLRTQARTSVVAGAKKYLTWQKAQAFGTAYPGSNGTYDWGSNSMVVNNQVVLATAFDITRDKKFSRAVLESMDYLLGRNALNNSYVTGYGTQFSSHQHSRWMVPPPNGSLAGGPNSLRSTWDPVMNTLFPESNPCTPQRCYVDDIEAWSVNEITVNWNSALSWIASFVADQGAAPQPVAPKITKQPTSVSGALKAKVTLSAAASGVPAPTVRWQRRAPGATAWKDVRGATSTTLKVVMTRAEHRAGYRAVFTSSAGSATTATATVTLRKARPVITSQPKDVTVRSGARATFRVAYDAYPAAHVQWWAKKGKTWVKVPGARAQTLTVKVTGTSSKHYRVVVKSSQGQVTSRTAHVTIRR

SEQ ID NO: 92 - protein, 927 aa, Herpetosiphon giganteus
MSADHSRTVIGIRSTRSFWARGLFVLWLIIALGCQQAIVPATSQIHAQPPQTVGNLLQNGDFSAGLAPWWATNTVTADAASGALVATITNAGSNPWDAIVGQNGLNLQAGQTYTVTFRIRAASTGTAVLKLQKEASPYTNYFSQDVALTTSDQTHQFVFTSGFDDAGAAFQFQMGGQGNNVLTIDDVQVLGETGPAEPSSNLVQNGNFVGGIEPWWTSGDLSIDTADGACLTINTAGSNPWDVQLGQHTIALQAGASYQLKFAAKSTTPVTLPVRLQQNGEPYTGYFAADPGLNAAWTEFVFTFTSAYSDAASLLFQIGGLGTPTICFNHVSLEMLETGIRVNQASYLPTMSKVASLAHPATEPLSWELHNAADTVVLSGQTTVYGEEAASAEHVHLIDFSSFQIVGEGYYLQVGSEQSYPFAIEDGLYLRLKYDALAYFYHNRSGIAITMPYAGDEQWTRPAGHIGVAPNQGDLAVTCFAGTDNQGQTWPGCDYELDVSKGWYDAGDHGKYVVNSGISVWTLLNQYERAQQRGAESLAQFADGSMNIPENNNGVPDLLDEVRWNMEFMLGMQIPTTAPVSKTGMVHHKVHDAQWTGLPMAPHDDPQMRYLYPPSTAATLNLAATAAQCSRIWRGIDEAFADRCLVAAERAWQAAVAYPNEFASDHFAGGGGYGDRTLNDEWYWAAAELYVTTGSATYREAIEESPYYLKLDIGGGSAMNWGSVASLGTLSLALVPNHLSSADRQTARAAVIAAADQFVAAQQASGYGIPYNPGSQYPWGSNSSILNNMIVIGLAGDFTGETSYADAISQGMDYLLGRNPLNRSYISGYGSVSLTNPHHRFWAKQINPEYPGTPPGVVAGGPNSGIEDPYAQAELTGCAALKCYVDHIDSWSTNEVTINWNSPLAWVAAYLDDYATGSVQYLPLITK

SEQ ID NO: 93 - protein, 991 aa, Ruminococcus sp.
MSKSFKRVTSGLLALSVVASMGSMNAFAGQQLGQNDFDSGIGLPWHTCETNPAKQEFDISGGTYNVKIVNNDGPEDRWDLQLRHRGLKIVSGHKYKLHWEVNSTSAGEIYTKIGDYDSVEAWHNNMDDSKGFDQTWDCVPIKAGDNEFEVEFTAQKSIDVAEWAFHYGGKGQYQKVDCFPNGTELKFDNLSLIDLTSDENDYDTTNEMGVVRPESNVRLNQVGYYTNLNKKASYVTDASSSLTFEVRDAKSGAVKYTGKTTVKGEDPDSGTGTTSGSKLKDSGKYVHILDFSSVKDAGTYKIFVKDTTGVSGTQVLLAKGANDTKLSGDKLMWTNPQTKKTYVMNESHDFVISDDVYGTQLLKDSINYYYQNRSGVPIESQYITSGDKSELSHTQYGHNPDTAYVQSEWKKAYASEFDGDKGKTIDGTGGWYDAGDHGKYVVNGGISVWTLQNAFEFTKKNGNTDKWTNKTIAIPENGDKNPDILDEARVELEWMFKMIDEDGFVYHKLHDHKWTGLATHCWDYEKKWKTTRIVKPATYAATFNMIACAAQAARLWEPYDKSFAQECLQHAEASWKAIYAKKDNWNVKEGDSSKDSYFAPLDQAIGGGAYGDTYVVDDAYWAACELYSTTGKSDYLSFLKSYKNPNDKTENDKAYSLTYNLGGGENNGSFSSFNWGCTSGLGTLSLYLSDKVSSSEKATITKSITAVADEYLKEMAKQGMGIPYRGATFTDAINIGEGVSVTGYEWGSNSFVINNAMVLAYAYDATKATASDKGGKYINGASEALDYIFGRNGLGFSYVSGYGDKTLEWPHHRFWSKGVDPAFPKAPNGVLSGGPGAGMQDPYIGGLGYKRGTLANQKCYVDSAEAWSVNEVTINWNAPLVWMTTFMLDEAPKFNGGTPDPVVVQCPTVKAEVQGKQFRLKWEAVKGATAYAVALYQNGAWRCKAQVSGSTLTYTSPKGVKSGKYQVAICPKVNGQWVNKSIKSKSITVTI

SEQ ID NO: 94 - DNA, 2580 bp, Aspergillus welwitschiae
  source 1..2580: mol_type = genomic DNA
atgaggttcactttgatcgaggcggtggctctgactgccgtctcgctggccagcgctgatgaattggcctactcccctccgtattacccctccccttgggccaatggccagggtgactgggcggaagcataccagcgcgctgttgatatcgtctcgcagatgacattggctgagaaggtcaatttgactacgggaactggatgggaattggaattatgtgttggtcagactggaggtgttccccgattgggaattccgggaatgtgtgcacaggatagccctctgggtgttcgtgactccgactacaactctgcgttccccgccggtgtcaacgtggccgcaacctgggacaagaatctggcttaccttcgtggccaggctatgggtcaggagtttagtgacaagggtgctgatatccaattgggtccagctgccggccctctcggtagaagtcccgacggcggtcgtaactgggagggcttctcccccgacccggccctcagtggtgtgctctttgcagagacaatcaagggtattcaggatgctggtgtggttgcaacggctaagcactacatcgcctacgagcaggagcatttccgtcaggcgcctgaagctcaaggctacggattcaatattaccgagagtggaagcgcgaacctcgacgataagactatgcatgagctgtacctctggcccttcgcggatgccatccgtgcaggtgccggtgctgtgatgtgctcgtacaaccagatcaacaacagctatggctgccagaacagctacactctgaacaagctgctcaaggctgagctgggtttccagggctttgtcatgagtgattgggcggctcaccatgccggtgtgagtggtgctttggcgggattggacatgtctatgccgggagacgtcgattacgacagtggcacgtcttactggggtaccaacttgaccatcagtgtgctcaacgggacggtgccccaatggcgtgttgatgacatggctgtccgcatcatggccgcctactacaaggtcggccgtgaccgtctgtggactcctcccaacttcagctcatggaccagagatgaatacggcttcaagtactactatgtctcggagggaccgtatgagaaggtcaaccagttcgtgaatgtgcaacgcaaccatagcgagttgatccgccgtattggagcagacagcacggtgctcctcaagaacgatggcgctcttcccttgactggaaaggagcgcttggtcgcccttatcggagaagatgcgggttccaatccttatggtgccaacggctgcagtgaccgtgggtgcgacaatggaacattggcgatgggctggggaagtggcactgccaactttccctacttggtgacccccgagcaggccatctcgaacgaggtgctcaagaacaagaatggcgtattcactgcgaccgataactgggctattgatcagattgaggcgcttgctaagaccgccagtgtctctcttgtctttgtcaacgccgactctggtgagggttatatcaatgtcgacggaaacctgggtgaccgcaggaacctgaccctgtggaggaacggcgacaatgtgatcaaggctgctgctagcaactgcaacaacacgatcgttattattcactctgtcggcccagtcttggttaacgagtggtacgacaaccccaatgttaccgctattctctggggtggtcttcccggtcaggagtctggcaactccctcgccgacgtgctctacggccgtgtcaaccccggtgccaagtcgcccttcacctggggcaagactcgtgaggcctaccaagattacttgtacaccgagcccaacaacggcaacggagcgccccaggaagacttcgtcgagggcgtcttcattgactaccgcggatttgacaagcgcaacgagactcctatctatgagttcggctatggtctgagctacaccaccttcaactactcgaaccttcaggtggaggttctgagcgcccctgcgtacgagcctgcttcgggcgagactgaggcagcgccgactttcggagaggtcggaaatgcgtcggattacctctaccccgatggactgcagagaatcaccaagttcatctacccctggctcaacagtaccgatcttgaggcgtcttctggggatgctagctatgggcaggatgcctcagactatcttcccgagggagccaccgatggctctgcgcaaccgatcctgcctgccggtggtggtgctggcggcaaccctcgcctgtacgacgagctcatccgcgtgtcggtgactatcaagaacaccggcaaggttgcgggtgatgaagttcctcaactgtatgtttctcttggcggccctaacgaacccaagatcgtgctgcgtcaattcgagcgtatcacgctgcagccgtcggaagagacgcagtggagcacgactctgacgcgccgtgaccttgcgaactggaatgttgagacgcaggactgggagattacgtcttatcccaagatggtgtttgtcggaagctcctcgcggaagctgccgctccgggcgtctctgcctactgttcac

SEQ ID NO: 95 - DNA, 2583 bp, Aspergillus luchuensis
  source 1..2583: mol_type = genomic DNA
atgaggttcactttgattgaggcggtggctctcactgctgtctcgctggccagcgctgatgaattggcttactccccaccgtattacccatccccttgggccaatggccagggcgactgggcgcaggcataccagcgcgctgttgatattgtctcgcagatgacattggctgagaaggtcaatctgaccacaggaactggatgggaattggagctatgtgttggtcagactggcggggttccccgattgggagttccgggaatgtgtttacaggatagccctctgggcgttcgcgactccgactacaactctgctttcccttccggtatgaacgtggctgcaacctgggacaagaatctggcatacctccgcggcaaggctatgggtcaggaatttagtgacaagggtgccgatatccaattgggtccagctgccggccctctcggtagaagtcccgacggtggtcgtaactgggagggcttctcccccgacccggccctaagtggtgtgctctttgcagagaccatcaagggtatccaagatgctggtgtggtcgcgacggctaagcactacattgcctacgagcaagagcatttccgtcaggcgcctgaagcccaaggttatggatttaacatttccgagagtggaagcgcgaacctcgacgataagactatgcacgagctgtacctctggcccttcgcggatgccatccgtgcgggtgctggcgctgtgatgtgctcctacaaccagatcaacaacagctatggctgccagaacagctacactctgaacaagctgctcaaggccgagctgggtttccagggctttgtcatgagtgattgggcggctcaccatgctggtgtgagtggtgctttggcaggattggatatgtctatgccaggagacgtcgactacgacagtggtacgtcttactggggtacaaacctgaccgttagcgtgctcaacggaacggtgccccaatggcgtgttgatgacatggctgtccgcatcatggccgcctactacaaggtcggccgtgaccgtctgtggactcctcccaacttcagctcatggaccagagatgaatacggctacaagtactactatgtgtcggagggaccgtacgagaaggtcaaccactacgtgaacgtgcaacgcaaccacagcgaactgatccgccgcattggagcggacagcacggtgctcctcaagaacgacggcgctctgcctttgactggtaaggagcgcctggtcgcgcttatcggagaagatgcgggctccaacccttatggtgccaacggctgcagtgaccgtggatgcgacaatggaacattggcgatgggctggggaagtggtactgccaacttcccatacctggtgacccccgagcaggccatctcaaacgaggtgctcaagaacaagaatggtgtattcaccgccaccgataactgggctatcgatcagattgaggcgcttgctaagaccgccagtgtctctcttgtctttgtcaacgccgactctggcgagggttacatcaatgtcgacggaaacctgggtgaccgcaggaacctgaccctgtggaggaacggcgataatgtgatcaaggctgctgctagcaactgcaacaacaccattgttatcattcactctgtcggcccagtcttggttaacgaatggtacgacaaccccaatgttaccgctattctctggggtggtctgcccggtcaggagtctggcaactctcttgccgacgtcctctatggccgtgtcaaccccggtgccaagtcgccctttacctggggcaagactcgtgaggcctaccaagattacttggtcaccgagcccaacaacggcaatggagccccccaggaagacttcgtcgagggcgtcttcattgactaccgcggattcgacaagcgcaacgagaccccgatctacgagttcggctatggtctgagctacaccactttcaactactcgaaccttgaggtgcaggttctgagcgcccccgcgtacgagcctgcttcgggtgagactgaggcagcgccaacttttggagaggttggaaatgcgtcgaattacctctaccccgacggactgcagaaaatcaccaagttcatctacccctggctcaacagtaccgatctcgaggcatcttctggggatgctagctacggacaggactcctcggactatcttcccgagggagccaccgatggctctgcgcaaccgatcctgcctgctggtggcggtcctggcggcaaccctcgcctgtacgacgagctcatccgcgtgtcggtgaccatcaagaacaccggcaaggttgctggtgatgaagttccccaactgtatgtttcccttggcggccccaacgagcccaagatcgtgctgcgtcaattcgagcgcatcacgctgcagccgtcagaggagacgaagtggagcacgactctgacgcgccgtgaccttgcaaactggaatgttgagaagcaggactgggagattacgtcgtatcccaagatggtgtttgtcggaagctcctcgcggaagctgccgctccgggcgtctctgcctactgttcactaa

SEQ ID NO: 96 - DNA, 2583 bp, Aspergillus sclerotioniger
  source 1..2583: mol_type = genomic DNA
atgaggttcagttggatcgaggtggcggctctgacagccgcctcggtggtcagcgctgatgaattggcgtactctccccctttctacccttctccctgggccaatggcaagggtgactggacagacgcgtacaagcgtgcggtcgacattgtttcgcagatgacactggccgaaaaggtcaatctgacaacgggaactggatgggaattggaaaagtgtgtcggtcagaccggcggtgttcctcgattgggaataccgggcatgtgtggtcaggatagtcctctgggtgttcgtgactctgactacaactcggctttccctgctggcatcaacattgccgcttcctgggacaaggacctcgcgtacctccgtggcaaggccatgggccaggagttcagtgacaagggtgctgatattcaattgggccccgttgctggtcccctcggtagaagtcccgacggcggtcgtaactgggaggggtttgctccagacccggccctgactggtgtgctctttgcagagaccatcaagggtatccaggatgctggcgtggttgcgacggctaagcacttcatcggctatgagcaagagcatttccgccaggcacctgaggctcaaggatatgggtacaacatcaccgagagtggcagcgcgaacatcgacgataagaccatgcacgagctgtacctctggccctttgcggatgctatccgcgcaggtgttggcgcggtcatgtgctcctacaaccagatcaacaacagctacggttgccagaacagctatgctctgaacaagctccttaagtctgaactggaattccagggcttcgtgatgagcgactgggaggcccaccacagtggagtcggtgctgctttggccggtctggacatgtctatgcccggagatgtcacctacgacagtggcacgtcttactggggtgccaacctgaccatcagtgtcctgaacggcacggttccccagtggcgtgttgatgatatggctgtccgtatcatggccgcctactacaaggtcggtcgggatcgtctgtggacgcctcccaacttcagttcatggacccgcgatgaatacggctacaagtactactattcctccgagggaccgtatgagaaggtcaaccagtttgtcaacgtgcagcgcaaccatagcgggttgatccgccgtattggagctgacagcacggttcttttgaagaacgagaacgttctgcccctgactggcaaggagcgcctggtcggtcttatcggagaagacgcgggctccaacgcttacggcgccaacggctgcagtgaccgtggatgcgacaacggtactctggctatgggctggggcagtggtacctcgaactacccttacttgatcactcccgagcaggcgatccaggctgaggtgatcaagaacaagggcaatgtatttgccgtgaccgacaactgggccattgaccagattgaggcactcgccaagcaatccagtgtctctcttgtctttgtcaacgccgactctggtgaaggttacatcgatgtcgatggcaacatgggtgaccgcaacaacattacgctctggaggaacggtgacaacgtggtcaaggccgctgccaacaactgcaacaacaccattgtcatcatccactccgtcggcccggtcctcgtcaccgagtggtacgaccaccccaatgtcaccggcatactgtgggctggcttgcccggccaggagtctggcaactcgctcgccgatgtgctctacggccgtgtcaacccgggtgccaagtcgcccttcacctggggtaagaccagggagtcctaccgcgactacctgatcaccgagcccaacaatggcgatggagccccacaggaagacttcaccgagggcatcttcatcgactaccgcgggttcgacaagcgcaatgagaccccaatctacgagttcggctatggcctgagctacaccaccttcaactactcgaatttgaacgtgcaggtcctgaatgcctcgtcgtacactccttcttctggcgagactgaggctgccccgacttttggagagatcggcaacgcgtcggactacctctaccctagcggactgaagaagattaccgacttcatctacccctggcttaacagcacggacctcctggaagcttccggcgacgccagctacggtctgaacgcgtcggagtatatccccgagggaggcaccgatggctctgcccagccgatcctggctgctggtggtggccctggcggtaaccccggtctgtatgacgagctgattcgcgtttcggcgaccatcaagaacaccggcaagcttgcgggtgacgaggttcctcaactgtacgtttcgcttggaggtcccgatgacgccaaaattgtgttgcgcaagttcgaccgcatctctctcaagccatcgcaggaggttgagtggagcacgactctgacacgacgcgacctggcaaactgggatgttgcggcgcaggactggaccatcacgtcttatcccaagacggtgtatgtcggtagctcttcgcggaagctgccgctccgcgcgtcactgcctactgtccaatag

SEQ ID NO: 97 - DNA, 2586 bp, Aspergillus tanneri
  source 1..2586: mol_type = genomic DNA
atgaggtttggttggtttgaggcggcggtcgtgaccgctgcctcagtggtcagtgcccaggatgatcttgctttctctcctccgtactatccttccccgtgggccaatggccagggagaatgggccgacgcgtatgaacgtgccgtcgacatcgtctcccagatgacgttggcggagaaggttaacctcactactggaacagggtggatgttggataaatgtgtgggtcagacaggaagtgttcccagactcggactattaagtctctgcttgcaagacagtcccttgggtattcggttttcggattacaattcggcattccccgcaggtgttaatgtcgccgcaacatgggacaagcaactggcgtacctccgcggtagggcaatgggtgaggaattcagcgacaagggaatcgacgttcaattaggccctgctgctgggcctctcggcagacaccccgatggtggtcggaactgggaaggtttctcccctgaccctgcccttaccggtgtacttttcgcagaaaccataaaaggcatccaggatgctggtgtgatcgcgacggccaagcattacattttgaatgaacaggagcatttccgtcaagttcccgaagcccttgcggctggattcaatatttcggactccctgagctccaatctagacgacaaaaccatgcatgagctgtacctctggcccttcgcagatgcagtgcgcgcgggtgtgggtgctgtgatgtgctcctacactcagatcaataacagttacggctgccagaatagcgaaactctgaataaacttctcaaggcggagcttggcttccagggttttgtcatgtcggactggagtgcgcaccacagcggtgtcggctctgccttggcggggctggatatgtcaatgcccggggatatcgccttcaatgacggcaattcgtacttcggggcaaacttgacgattggcgtcctcaacggaaccatccctcagtggagagtggacgacatggctgtccgcatcatggctgcttactataaggttggccgggatcgccaccacgcgcctcccaacttcagctcatggaccagggacgaatacggctacgagcacgctgcagtttcggagggcgcgtatgaaagggtgaaccaatttgtcgacgtgcagcgggaccatgcggaaatcatccgtcgtgtgggtgccgagagcattgttctcttgaagaacgagaacgctctgccgttgagtgggaaggagaagaaggtcgttatcctcggagaagatgcgggatccaacccttggggtgccaatggctgcgaaaaccgtggatgtgacaatgggacccttgccatggcttggggcagtggcactacagagttcccctacctcgtcactcccgaacaggcgatccagaacgaggtcctccgaggtcgtggtaatgtatttagtgtcaccgacaacggggctctgagtcagatggcggcgcttgcctctcaatctagtgtcgctcttgtctttgtcaatgccgactcgggtgagggattcatcaccgtggatggaaacgagggagatcgcaagaacctcactctctggaagaacggagagaacgtgatcaaaaccgctgcccaaaactgcaacaacaccgtcgtgatcatccactccgtcggagccgttctggttgacgaatggtatgaccaccccaatattaccggcattctttgggcgggtctgcctggtcaggagtccgggaactccctcgccgacgtgctgtacggccgtgtcaaccctggcggtaagacccccttcacctggggtcgaacccgcgaatcgtatggggcacccctggtcaccgatgcgaacaacggccacggtgcgccccagtcggacttcgaagaaggggtgtttattgactaccgtcatttcgacaagttcaacgagactcccatctacgagtttggctacggccttagttacaccactttcggctactcgggactgcaaattgagcccctgaacgcgcccaagtacgtccccaactccgggaagaccgaagcggcgccaactttcggcgatgttgcagaggtctcagactatgtgtatccaaagggactgcggagaatccgtgagttcatctacccctggctgaattccaccaatctgaagaagtcctccggcgatgccacttatggaggggaggactcgacgtacatccccgatgacgccaccgatggttctcctcaggaccttctgcctgccagcggtggtcccggaggcaatccaggtctctaccaggatctggtgcgggtgtcggttacgatcacgaacacgggcaacgtggccggctacgacgtgcctcagctttatgtttcccttggtggccccaacgagccgaaggtggtactccgcaaattcgatcgatttaagctggatccgtcgcagcaggtggtgtggtcgaccacgctgaatcgtcgcgacctgtccaactgggacgtgacggggcaggactgggtcatcaccgagtatcccaagaaggtcttcgttggtagctcgtcccggaaattgccgctacatgcttccttgccggagatggagtaa

SEQ ID NO: 98 - DNA, 2586 bp, Aspergillus glaucus
  source 1..2586: mol_type = genomic DNA
atgaagctcggctggttggagtttgttgccgtcacggcctcggtggcccaggctaaggacctcgcgtactctcccccctactatccctcgccatgggccgatggtcaacccgccgaatggtccaacgcgtacaaacgcgccgtcgacatcgtctccaacatgactttggcggaaaaggtcaacttgaccactggtactggctggcaattggaagaatgtgttggtcagactggtagtgttccacgacttggtatctggggtatctgtttgcaggactcgcctcttggtatccgttatggcgatcacagctctggcttccccgctggtctcaacgtcgccgcgacctgggaccgcaagctcgcctacctccgtggtgaggctatgggtcaggaattcagcgacaaaggaattgacgtccaattgggtcccgttgctggccctatcggcagatctcccgacggtggtcgcaactgggagggtttcgcccccgacccggttctgaccggtgtgctcatggccgagactatcaagggtattcaggatgccggtgtcatcgctactgctaagcactacatcggcaacgagcaggagcatttccgtcaggtttccgaggctctcgactatggttacaacatcactgagactgccagctccaacatcgacgacaagaccatgcacgagctctacttgtggcctttcgccgatgctgttcgtgctggtgtcggttccgtcatgtgctcgtacaaccagatcaacaacagctatggttgccagaacagtcatttgctgaacaagctgctcaagcacgagctgggcttccagggcttcgttatgaccgactggggtgctcaccatagcggtgtcgcctccacccttgctggtactgacatgtcgatgcccggtgatatctcgtttgacgatggtatgtcgtacttcggtcccaacctgactgttgctgttctcaacggtactgttcccgagtggcgtgtcgacgacatggccgttcgtatcatgtctgctttctacaaggttggccgtgaccgccagcgcacgcctcccaacttcagctcctggaccaacgacgagtacagctacgcccactccgccgtccaggaaggctggaagcaggtcaaccagcacatcaatgtccagcgcaaccactccgagatcatccgtgaggttggcgaagccagcactgttctcttgaagaacaagggtgctcttcctctgactggcgacgagggctccgttggtattctaggcgaagatgccgggtccaacgcgtatggtgccaacggctgcgaggaccgtggatgcgacaatggtacccttgcgatggcctggggtagcggttccgccgaattcccttacctggttactcccgagcaggctatccagaacgagattctcaaccgggacgtcaagcaccccgtcttcgccgtcactgacaactgggctttggatcagatggcctccattgcctctcaatctgatgtttctcttgtcttcgtcaacgccgatgctggtgagggtttcctcgtcgtggatggtaatgagggtgaccgcaataacatcaccctctggaagaacggtgagaatgtcatcaagactgtcagcgagaactgcaacaacaccattgtgatcatgcacaccgtcggacccgtcctcatcgaccagtggtacgacaaccccaacatcaccgccattgtctgggctggtctgcctggccaggaatccggaaacgccattgccaacgttctctacggccgtgtcaaccctggtggcaagagccccttcacctggggtaagagccgcgaggcatacggtgccccgctcctcaccgagacaaacaacggcatcggcgctcctcaggtcgactttactgagggtcagttcatcgactaccggcgcttcgacaagtacaacgagacccctatctacgagtttggctacggtctgagttataccaccttcaagtactccaacctccacgtccaggccctgaacgcctccaagtacgttcccaccactggcaagaccagtgccgcgcccaagcttggtgaggttggcaaggcttcgaactacgtcttccccaatggattcgagcgcaccaccaagttcatctacccctggttgaactcgaccgatctgaagaagtccgccaacgaccccgagtacggcctcgagatctcaaagtacatccccgagaacgctcaggacgaatcctcccaggcccgtttgccagccagcggcggccaaggaggcaacaccggtctctacgacgaactcttccgcgtctccgccacgatcaagaacaccggcaaggtcgcaggtgacgaagttccccagctgtacgtctctctcggcggccccaacgagcccaaggttgtcctgcgcaactttgaccgcatcgccctccagcctggtcaggaggtcgtgtggtcgaagactcttacccgtcgtgacctttccaactgggacgttgccgctcaggactgggctatcacgcagcataccaagaaggtgtttgttgggagctcgtcgcgcaagctgcctttgcgggcttcgttacctcgcgtgcagtag

SEQ ID NO: 99 - DNA, 2589 bp, Penicillium argentinense
  source 1..2589: mol_type = genomic DNA
atgaagctcgggttgctcgaagccgccgtcttgacggccgcctcggccgccactgcgtcggacctggcatactcgcccccgtactatccgtccccgtggatgaccggcgagggcgactgggccgaggcctaccgccgcgccgtcgacttcgtctccaacctcacactggctgaaaaggtcaacttgaccactggtgcgggctgggagcaagaacgctgcgtcggggagacgggtggtattccccgactgggaatgtggggcatgtgcttgcaagactcgcccctgggcatccgcgacagtgactacaactccggtttcgccgcgggcgtcaatgttgccgccacatgggacaagagactcgcctaccagcgtggtctggcgatgggcgaggagcaccgcgacaagggtgtcgatgtgcagctcggtcctgtggccggcccactgggacgaagccccgatggcggccgtggctgggaagggttctcgcctgatcctgtcctcaccggtgttatgatggcggagactatcaagggtattcaggatgctggtgtcattgcttgcgccaagcactatatcggaaacgagcaggaacacttccgccagtcgggcgaggctcagggctatgggtacaacatcactgagagtgtgagctccaacatcgacgacaagactatgcacgaactgtacctctggccctttgtcgactctatccgcgccggtgtcggctctgtcatgtgctcgtacaatcagatcaacaacagctatggatgccagaacagcgagaccttgaacaggttgctcaaggctgagctgggtttccagggcttcgtcatgtcggattggggtgcgcaccatagcggtgtcagcgctacccttgctggtctggatatgtccatgcccggtgatgtcatcctcggtagcccgtactccttctggggcacgaacctgaccatctccgtgctgaacggtaccgtccccgaatggcgtatcgacgatatggccgtccgcatcatgtcggcctactacaaggtcggccgtgaccgtgtccgtgtccctcccaacttcagctcctggactcgtgatgagtatggctttgagcacttcatggtcagcgagaactacatcaagctcaacgagcgcgtcaatgtccagcgcgaccacgccgcgggtatccgcaagcttggctctgacagcaccgttctgctgaagaacaagggtgctctgcccttgactcacaatgagaagttcgttggtattctgggtgaggatgctggttccaaccctgctggcgctaatggctgtgcggaccgtggttgcgatgacggcacccttgctatgggctggggtagtggtactgctaacttcccttacctgattacccccgagcaagccattcagaacgaggttctgaactatggcaatggccagacaaatgtgtttgccgtgaccaacaacaccaacacggagcagattgctgccattgctgcgcagtcgagcgtcgctctcgtcttcgtaaacgccgactccggcgaaggcttcatcaatgtcgacggcaacgagggtgaccgcaagaacctaaccctctggaagaacggcgaacacctcatcaaaacagctgcggcaaactgcaacaacaccgtcgtcatcatgcacacccccggcgccgtcctaatcagcgactggtacgagcacgacaacatcaccgccatcctctgggccggtctaccgggccaagaaagcggccgcagtatcacagacattctctacgggcgcgtgaacccaggcggcaaaacacccttcacctggggaaagactcgcaaagactacggcgcaccgctcctcacacaacccaacaacggccatggtgcgccgcagcaagacttcacagagggagtcttcatcgactaccgccgcttcgacaaggagcaggaagagcctgtctacgaattcgggtacggactcagctacacgaatttcgaattcagcgatctgcacatcaagtcccttaacgcagacaagtatgtcccgacaacggggacaaccaagcctgcccccgtcttcggcaaaatcggccaggcatccgactatctcttcccgaagggcatccaccgtgtcacccaatacctctacccctacctgaacagcacaaacctcaagtcctcctccggcgacccctactacggcgctaagacagaggagtacatccccgccggcgcaaccaacggctccgcgcaaacccgcctcccatccagcggtgccaacggcggtaatgcgggtctcttcgaggatctgtaccaggttaccgtgacaattaccaacaccggctctgtgcagggcgacgaagttccccagttgtacgttagtctgggcggggagaataaccccgttaaggtcctgcgtgcgtttgacaggatcaccattgcgcctggccagaaggcacagtggactactactcttacccgccgcgatttgtcgagttgggatgttgcaaaacagaattgggtagttactcctgcgcagaagaaggtgtatgttggaaattcgtcgcggagacttccgcttgagtcggagttgcccgcttcgaaatga

SEQ ID NO: 100 - DNA, 2583 bp, Aspergillus wentii
  source 1..2583: mol_type = genomic DNA
atgaggttcggttggttggaggtagccgctcttacggctgcctccgtggtcagtgccaaggatgacctggcattctcccctcccttctacccctctccgtgggcaaacggtgagggtgaatgggcagactcgtacaagcgcgctgtcgagtttgtttcgaacttgaccttggctgagaaggtcaaccttacaactggttctggctggcagcaagagagatgtgttggtgagacgggcgaagtttctagacttggattctggggtatttgtttgcaggattctcccctgggtattcgttttggcgaccactcctccggcttcccctccggcctcaacgttgctgcaacctgggacaaaaagcttgcctacctccgtggtaaggcaatgggcgaggaattccgtgacaagggcattgatgttcaactgggacctgtcgctggtccccttggcgctttccctgatggtggtcgaaactgggagggtttcgctcccgaccctgtgctgactggtttcctgatgtcggagactatcaagggtatccaagatgctggtgttattgctactgctaagcattacatcggtaatgagcaggagcatttccgccaaagcggcgaggctaagggttatggttacaacattactgaaagttcgagctcaaacattgatgacaagacaatgcacgagttgtatctctggccttttgctgatgccgttcgtgctggtgtcggcgcgttcatgtgctcatacaaccagatcaacaacagctatggctgctccaacagctacctgatgaacaaactcctcaagtctgaactaggcttccaaggatttgtcatgagtgattggggtgcacaccacagcggtgttggcgctgctttggctggtttggatatgtccatgcctggtgacactgtcatgggcgacccctacaccttctggggaaccaacctgaccatctcggttctcaacggcaccgtccccgagtggcgtgtggatgacatggccgtccgtatcatggctgcttactacaaggtgggccgcgatgaggttcgcacccctcccaacttcagctcgtggaccaccgaagaattcggatacgcgcattatgctgctcaggaaggatacgagaagaccaactggaatgtcaatgtccgccgcaaccacgccaaggtcatccgcgaaatcggttcggccagcacggttcttctgaagaacaatggcgtgctccccctgaccggtgatgaggactatgtcggaattctgggcgaggacgccggagctaacccctatggtgccaacggctgtgaagaccgtggctgcgacaatggtactctcgctatggcttggggcagtggtagtgcggaattcccttaccttgtgactcccgagcaggccatccagaacgaggttctcaagggcgagggctctgtgttcgctgtgaccgataactgggctctcgagcagatggcttctattgcttcgcagtcctctatctctctcgtctttgtcaatgccgactccggagaaggtttcctcaatgtggacggaaacatgggtgaccgcaagaacttcaccctctggaaaaatggcgagaatgtgatcaagactgtcactgagaactgcaacaacaccgttgtggtcatgcacactgttggcccggtcctgatcaaggactggtatgacaaccctaacatcactgccattgtctgggctggtttgcctggccaggaaagcggaaactctctcgctgatgtgctttacggccgtgtcaaccctagtggcaagagcccattcacctggggcaagactcgcgaggcttatggtccttctctgctcaccacccagaacaacggcaatgacgctccccagcaagatttcacccagggtgtcttcattgactaccgccgatttgacaagttcaacgagactcccatctacgagttcggatacggtctgagttacaccaccttcaactactccaacctcgaagttcgatccctgaatgcatcgcggtacaccccaaccactggcaagactgatgccgcgccttctctgggcgaggctggcaaggcctcggactttttgttccccaagggactgaaccgcatcatcggctacatttatccgtggttgaactcgaccgacctgaagtcggcgtccggagagaagaactatggcatgaaggcttctgagtacattcccgagggagccaccgatggatccgcccaggagctcctgccggccggcggtggccctggtggtaaccctggtctgtatgaagacctcatcgaggtttctgctactattaccaacactggcaaggttgctggtgacgaagtcccacagctgtatgtctcccttggtggtgccgacgaccccgttcttgtcctccgtcagttcgaccgtctccacatcgaggctggaaagcaggcagtgtggaagacgactcttacccgccgtgacctcgccaactgggacgttgcggcgcaagactggaccatcacgaagagcgcgaagaaggtgtacgtgggcagctcttcgcggaacctgcccctcaaggccaagcttcccaccgttcagtag

SEQ ID NO: 101 - protein, 916 aa, Penicillium vulpinum
MSTIIAPVSKELFEYLRTHAFPEICDSFLPHAFFYQQRFLCCLCEILSSIFTAMKFEWLTVAGLTAAANANPLAYSPPYYPSPWMTGAGEWSDAYTRAVDFVSNLTLAEKVNLTTGAGWEQERCVGETGGIPRLGMWGMCMQDSPLGVRLSDYTSGFPSGINVAATWDKRLAYQRGMAMGEEHRDKGVDVQLGPVAGPLGKYPEGGRNWEGFSPDPVLTGVMMAETIKGMQDAGVIACAKHFIGNEQEHFRQSGEAQGYGYNISESVSSNIDDKTMHELYLWPFVDSIRAGVGSIMCSYNQINNSYGCANSYALNKLLKGELGFQGFVMSDWGAHHSGVSSTLAGLDMSMPGDTYLGSPSSFWGANLTISVLNGTVPEWRIDDMAVRIMAAYYKVGRDRFRTPPNFSSWTRDEYSFEHAAVSEGWAKVNERVNVQRDHAQIIRKIGSDSTVLLKNKGGALPLTHGEKFISILGEDAGSNAYGANGCGDRGCDNGTLAMGWGSGTANFPYLITPEQAIQNEVLEYSVGKTSVFAVTDNWALTEMAALASQADVALVFVNADSGEGYINVDGNEGDRKNLTLWKNGEEIIKTASQHCNNTIVVIHSTAAVLISDWYDNDNITAIVWAGLPGQESGRSLVDVLYGRINPGGKTPFTWGKTRKDYGPPLLTVPNNGADAPQDNFEDGVFIDYRRFDKDNTEPIYEFGYGLSYTNFSFSDLKVTPLASSEYNEYKATTGKTKKAPVLGKAGKVSDNLFPEGIKPVRQYLYPWLNSTDLRASSGDPAYGMDSKDYLPEGATDGSPQDLLPSSGASGGNPDLFKNLYQVTATITNTGSVTGDEVPQLYVSLGGDDEPSKVLRQFDRVTIAPGQTLQWTTTLTRRDVSNWDVASQNWVISDAPKKVYVGNSSRKLPLSADLPPI

SEQ ID NO: 102 - protein, 1230 aa, Aspergillus terreus
MKLSILEAAALTAASVVSAQVTFTLPVNLVLDGALTVRKQDDLAYSPPYYPSPWADGHGEWSNAYKRAVDIVSQMTLTEKVNLTTGTGWELERCVGQTGSVPRLGIPSLCLQDSPLGIRMSDYNSAFPAGINVAATWDKTLAYQRGKAMGEEFSDKGIDVQLGPAAGPLGRSPDGGRNWEGFSPDPALTGVLFAETIKGIQDAGVIATAKHYILNEQEHFRQVGEAQGYGFNITETVSSNVDDKTMHELYLWPFADAVRAGVGAVMCSYNQINNSYGCQNSLTLNKLLKAELGFQGFVMSDWSAHHSGVGAALAGLDMSMPGDISFDSGTSFYGTNLTVGVLNGTIPQWRVDDMAVRIMAAYYKVGRDRLWTPPNFSSWTRDEYGFAHFFPSEGAYERVNEFVNVQRDHAQVIRRIGADSVVLLKNDGALPLTGQEKTVGILGEDAGSNPKGANGCSDRGCDKGTLAMAWGSGTANFPYLVTPEQAIQNEVLKGRGNVFAVTDNYDTQQIAAVASQSTVSLVFVNADAGEGFLNVDGNMGDRKNLTLWQNGEEVIKTVTEHCNNTVVVIHSVGPVLIDEWYAHPNVTGILWAGLPGQESGNAIADVLYGRVNPGGKTPFTWGKTRASYGDYLLTEPNNGNGAPQDNFNEGVFIDYRRFDKYNETPIYEFGHGLSYTTFELSGLQVQLINGSSYVPTTGQTSAAQTFGKVEDASSYLYPEGLKRISKFIYPWLNSTDLKASTGDPDYGEPNFEYIPEGATDGSPQPRLPASGGPGGNPGLYEDLFQVSVTVTNTGKVAGDEVPQLYVSLGGPNEPKRVLRKFERLHIAPGQQKVWTTTLNRRDLANWDVVAQDWKITPYAKTIFVGTSSRKLPLAGRLPRVHLVSNQKMKHDVIFFQTPRLLPRQRLNYSQSLRDTAHSHGTTLPSVRPSPETKKTNHQPHGTRRKTEKKNMATTARASSPPGTRPFQPPTAALLVYPATLIIGSLFSVLSPTAQGARASASDDAATAAAPVNYFARKNNIFNVYFVKIGWVWTTLAFAAILLTQPAYTAPSAQRPRRLAQAAARYALATLVWWLTTQWFFGPAIIDRGFVLTGGKCEARAEGTHAASSLPVSPLGMVSAAACKAAGGAWTGGHDVSGHVFMLVLATAMLGFEMGGVFGVEGGKGVGVWSRRFVGAVLGLSWWMLLMTAIWFHTWFEKLTGLLIALGTVYTVYILPRRVVPWRNVVGIPGV

SEQ ID NO: 103 - protein, 882 aa, Stachybotrys microspore
MALASTFLSLYIFLFAYIFTGFACADPDSLYQRQDDGLLTSPPDYPSPWADPEANGWEQAYAQARDFVSQLTLLEKVNLTTGVGWMSERCVGNTGSIPRLGLRGLCLQDGPLGMRFTDYNSAFPAGVVAGATWSRHLWHDRGRMMGEEQYGKGSDVLLGPASGPIGRAPTGGRNWEGFSVDPYHSGVAMAQVVRGIQGAGVIATAKHFVANEQEHFRQAPEAVGYGFNITESLSSNLDDKTLHELYAWPFQDAVRAGVGSIMCSYNQINNSYGCQNSKLLNGILKDEYGFQGFVMSDWQAQHAGAASAAAGLDMSMPGDTVFNTGYSFWGGNLTLGVINGTVPEWRIDDMALRIMAAFFKVGRTVGGQPDINFSSWTRETLGYIHPMAQENLERVNFQVDVRGNHANHIRESAAKGSVILKNNGVLPLSSPKFVVVIGEDAGGNPAGPNGCPDRNCDNGTLAMAWGSGTAQFPYLVTPDQALQRQALEDGTRYESILANNQWTAVQNILREPNTTTIVFADADSGEGFIDVDGNRGDRRNLTLWKDGDALIKNVSSLTSNVIVVLHTVGPVLLTEWYDNPNITAIVWAGVPGQESGNSLTDILYGRRSPGRSPFTWGRTRESYGADLLYEPNNGEGAPQQDFSEGVFIDYRHFDRETAADPENTPIYEFGYGLSWSTFEYSNLQVAKRNVRPYQPTTGNTISAPVFGAPVDPDLSQYTFPPGIRYIYRFIYPYLNTSSSGEDASGDPEYGQEADQFLPPGAIDGSPQPRHPAGGQPGGNPQLWDVLYTVTATITNTGDRMTDEVPQLYISHGGDGEPVRVLRGFDRIERIAPGESVQFRADLTRHDLSNWDVVSQNWVITDDEKTVWVGSSSRNLPLAAPLR

SEQ ID NO: 104 - protein, 857 aa, Rasamsonia emersonii
MRNGLLKVAALAAASAVNGENLAYSPPFYPSPWANGQGDWAEAYQKAVQFVSQLTLAEKVNLTTGTGWEQDRCVGQVGSIPRLGFPGLCMQDSPLGVRDTDYNSAFPAGVNVAATWDRNLAYRRGVAMGEEHRGKGVDVQLGPVAGPLGRSPDAGRNWEGFAPDPVLTGNMMASTIQGIQDAGVIACAKHFILYEQEHFRQGAQDGYDISDSISANADDKTMHELYLWPFADAVRAGVGSVMCSYNQVNNSYACSNSYTMNKLLKSELGFQGFVMTDWGGHHSGVGSALAGLDMSMPGDIAFDSGTSFWGTNLTVAVLNGSIPEWRVDDMAVRIMSAYYKVGRDRYSVPINFDSWTLDTYGPEHYAVGQGQTKINEHVDVRGNHAEIIHEIGAASAVLLKNKGGLPLTGTERFVGVFGKDAGSNPWGVNGCSDRGCDNGTLAMGWGSGTANFPYLVTPEQAIQREVLSRNGTFTGITDNGALAEMAAAASQADTCLVFANADSGEGYITVDGNEGDRKNLTLWQGADQVIHNVSANCNNTVVVLHTVGPVLIDDWYDHPNVTAILWAGLPGQESGNSLVDVLYGRVNPGKTPFTWGRARDDYGAPLIVKPNNGKGAPQQDFTEGIFIDYRRFDKYNITPIYEFGFGLSYTTFEFSQLNVQPINAPPYTPASGFTKAAQSFGQPSNASDNLYPSDIERVPLYIYPWLNSTDLKASANDPDYGLPTEKYVPPNATNGDPQPIDPAGGAPGGNPSLYEPVARVTTIITNTGKVTGDEVPQLYVSLGGPDDAPKVLRGFDRITLAPGQQYLWTTTLTRRDISNWDPVTQNWVVTNYTKTIYVGNSSRNLPLQAPLKPYPGI

SEQ ID NO: 105 - protein, 856 aa, Penicillium waksmanii
MKLGWLEAATLAAASVASASDLAYSPPHYPSPWSTGDGEWAEAYRRAVEFVSNLTLPEKVNLTTGAGWEQEKCVGETGGIPRLGMWGMCMQDSPLGIRDSDYNSGFAAGVNVAATWDKRLAYQRGLAMGEEHRDKGVDVQLGPVAGPLGRSPDGGRVWEGFSPDPVLTGVMMAQTIKGIQDAGVIACAKHFIGNEQEHFRQAGEAQGYGYNISESVSSNIDDKTMHELYLWPFVDSVRAGVGSVMCSYNQINNSYGCSNSYTLNKLLKGELGFQGFVMSDWGAHHSGVGDALAGLDMSMPGDVILGSPYSFWGTNLTISALNGTIPEWRLDDMAVRIMAAYYKVGRDRVRVPPNFSSWTRDEYGYEHFIVSENQIKLNERVNVQRDHASGIRKLGSDSTVLLKNKGALPLTHNERFVAILGEDAGSNPAGANGCSDRGCDDGTLAMGWGSGTANFPYLITPEQAIQNEYLNYGNGQTNVFAVTNNSNTEQIAAMASQATVSLVFVNADSGEGYINVDGNEGDRKNLTLWKNGEQLIKTAAENCNNTIVIMHTPSAVLVGDWYDNENITAILWAGLPVSPIFSMVASTPARRPPFTWGKTRDAYGAPLLTKPNNGQGAPQQDFSEGVFIDYRQFDKADEEPIFEFGFGLSYTKFEFSDVHVTPLKADKYTKTTGRTKPAPVLGKIGEASDYLFPSGIKRVTQYLYPWLNSTNLKESSGDPYYGEKAEKYIPAHARDGSAQQLLPASGPSGGNAGLFEDLFQVTATVTNTGSVVGDEVAQLYVSLGGEGDPVKVLRAFDRITIAPGQNAQWTTTLTRRDLSNWDVASQNWVISDAPKKVYIGNSSRHLPVSIELPSTK

SEQ ID NO: 106 - protein, 801 aa, Aspergillus udagawae
MTLAEKVNLTTGTGIFMGPCAGQTGSALRFGIPNLCLHDSPLGVRNSDHNTAFPAGITVGATFDKDLMYDRGAEMGEEFRGKGINVLLGPSVGPIGRKPRGGRNWEGFGADPSLQAIGGAQTIKGIQSKGVIATIKHYIGNEQEMYRMSNIGQRGYSSNIDDRTLHELYLWPFAEGVRAGVGAVMAAYNDVNSSACSQNSKLLNEILKDELGFQGFVMTDWLGQYGGVSSALAGLDMAMPGDGAVPLLGDAYWGSELSRSILNGSVPVSRLNDMVTRIVATWYKMGQDGDYPLPNFSSNTQDATGPLYPGALFSPSGVVNQYVNVQADHNITARAIARDAITLLKNDDNILPLRKNDSLKIFGADAGPNPDGLNSCADQGCNKGVLTMGWGSGTSRLPYLVTPQQAIANISSNATFYITDSFPSNLAVGSGDIALVFINADSGENYITVEGNPGDRTSAGLNAWHNGDKLVKDAAAKFSKVVVVIHTVGPILMEEWIDLPSVKAVLVAHLPGQEAGWSLTDILFGDSSPSGHLPYTIPRSESDYPSSVGLLSQPLVQIQDTYTEGLYIDYRHFLKANITPRYPFGHGLSYTTFSFSQPTLSVRTAVGSAYPPTRAPKGPTPSYPTTIPNPSEVAWPKNFDRIWRYLYPYLDDPAGAAKNSSKTYPYPTGYTTVPKPAPRAGGAEGGNPALFDVAFAVSVTVTNTGKRPGRAVAQLYVELPNTLGVETPSRQLRQFAKTKVLAPGARETLTMEITRKDISVWDVVVQDWKAPVQGQGVKFWLGESVLDMRAVCEVGGACTII

SEQ ID NO: 107 - protein, 753 aa, Aspergillus transmontanensis
MLLTVDAQRTIIRLSLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTIHELYLWPFADAVRAGVGAVMCSYNQINNSYGCENSETLNKLLKAELGFQGFVMSDWTAHHSGVGAALAGMDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLINEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASQYTPTSGMTEAAKNFGEIGDASEYVYPEGLERIHEFIYPWINSTDLKASSDDSNYGWEDSEYIPEGATDGSAQPRLPASGGAGGNPGLYEDLFRVSVKVKNTGNVAGDEVPQLYVSLGGPNEPKVVLRKFERIHLAPSQEVVWTTTLTRRDLANWDVSAQDWAVTPYPKTIYVGNSSRKLPLQVSLPKAQ

SEQ ID NO: 108 - protein, 694 aa, Penicillium oxalicum
MKLEWLEATVLAAATVASAKDLAYSPPFYPSPWATGEGEWAEAYKKAVDFVSGLTLAEKVNITTGAGWEQERCVGSVMCSYNQINNSYGCSNSYTLNKLLKGELGFQGFVMSDWGAHHSGVGDALAGLDMSMPGDVILGSPYSFWGTNLTVSVLNSTIPEWRLDDMAVRIMAAYYKVGRDRHRTPPNFSSWTRDEYGYEHFIVQENYVKLNERVNVQRDHANVIRKIGSDSIVMLKNNGGLPLTHQERLVAILGEDAGSNAYGANGCSDRGCDNGTLAMGWGSGTANFPYLITPEQAIQNEVLNYGNGDTNVFAVTDNGALSQMAALASTASVALVFVNADSGEGYISVDGNEGDRKNMTLWKNGEELIKTATANCNNTIVIMHTPNAVLVDSWYDNENITAILWAGMPGQESGRSLVDVLYGRTNPGGKTPFTWGKERKDWGSPLLTKPNNGHGAPQDDFTDVLIDYRRFDKDNVEPIFEFGFGLSYTKFEFSDIQVKALNHGEYNATVGKTKPAPSLGKPGNASDHLFPSNINRVRQYLYPYLNSTDLKASANDPDYGMNASAYIPPHATDSDPQDLLPASGPSGGNPGLFEDLIEVTATVTNTGSVTGDEVPQLYVSLGGADDPVKVLRAFDRVTIAPGQKLRWTATLNRRDLSNWDVPSQNWIISDAPKKVWVGNSSRKLPLSADLPKVQ

SEQ ID NO: 109 - protein, 707 aa, Histoplasma mississippiense
MLLPIAFASLAVATEHLTESPPYYPSPWASGQGGWEDAVERARDFVSQLTLVEKVNLTTGVGWMQENCVGQVGSIPRMGLHSLCMQDGPLGIRFADYVSAFPAGVNVGATFSKELAYLRGKAMGEEHRDKGVDVVLGPVVGPLGRSPDGGRNWEGFSPDPVNSGLLVAETIKGIQSASVIACVKHFIGNEQERFRQGPEAQGYGFDISESSSSNIDDVTMHELYLWPFADAVRAGVGSVMCSYNQINNSYGCGNSYTQNKLLKAELGFQGFIMSDWQAHHSGVGSALAGLDMSMPGDTVFGTGRSYWGPNLTIAVANGTIPEWRVDDMAVRIMAAYFKVGREAAKVPVNFNSWTRDEYDYTHALVKEGYGKVNERINVRAKHASIIRQVGAASVVLLKHTGSLPLTGLEKNAAVIGEDAGPNLWGPNGCPDRRCDNGTLAMGWGSGTADFPYLVTPAEAIQNEILSKGEGSVFPIFDNWASDQIKSAASQATVSLVFVNADSGEGFISVDGNEGDRKNLTLWKGGDELIQTVASYCNNTVVVIHSTGPVLVGEWNEHPNITAILWAGLPGQESGNSIADVLYGKVNPGGRSPFTWGRTAEDYGASILKEPNEGNGAPQVDFTEGIFTDYRAFDKADIKPIYEFGFGLSYTSFSYSDLNVEVPPWTLITDYRTKITSLLAQPMALHKNFFLLVGDPVEILVFMKYYIV

SEQ ID NO: 110 - protein, 777 aa, Exophiala dermatitidis
MHELYLWPFSDAVRAGTGSVMCSYNQINNSYGCANSYTMNHLLKNELDFQGFIMSDWQAQHSGVGTALAGLDMTMPGDTLFNTGRSYWGTNLTIAVINGTVPEWRVDDMATRIMAAYYYVGRDTHYTPTNFYAWSRNTYDKIHQVDPQSPIGLVNEHINVQDRHRDIVRQVGQASNVLLKNTGGLPLTGKERQVGIFGYDAGSNPWGANGCSNRGCDNGTLAMGYGSGTAEFPYLVTPEQAITQHVLTQTDGEVFAILDNYADAQIKSLASTADVALVFANAQSGEGFITIDGNTGDRNNLSLWLGADRLIHNVTKYNKNVIVVMHTVGPVNVSAWYDNENVTGIIWAGLPGQESGNAIVDALYGLINPGGKLPFTIGRNREDYGTDILYEPNNGQFNAPQSLFSEGVFIDYRHFDQYNIEPIYEFGFGLSYTTFEYSNLVITPGNPAPYTPTSGQTEPAPVLGNASTDPSQYVFPNGTITYRPYLYIYPYLNSTDLRASSDDTDYGLPTDQYVPPGATDGSPQPLLPAGGAPGGNAGLYEVVATVSATITNTGSVEGDEVAQLYVSLGEGEPPKVLRGFDRLTIAPGASTTFTANLTRRDVSVWDTVSQNWVQVSNPTIYVGTSSRKLPLSGVLSSSGGGSGAQSSSSASGGSGGGSSSGWGYGQSSTASGSAPAPVSQLSDGQPQVPTGRPVTQVSDGQPQAPTGNPVSQISDGQPQAPAHTGGAPVSQISDGQPQVPTGTGPAVTQISDGQPQNPTGGPAVSQLSDGQPRATTA

SEQ ID NO: 111 - protein, 860 aa, Aspergillus welwitschiae
MRFTLIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSEETQWSTTLTRRDLANWNVETQDWEITSYPKMVFVGSSSRKLPLRASLPTVH

SEQ ID NO: 112 - protein, 860 aa, Aspergillus eucalypticola
MRFTLIEAVALTAVSLASADELAYSPPYYPSPWANGQGDWAQAYQRAVDIVSQMTLAEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAQTFGEVGNASDYLYPDGLQRITKFIYPWLNSTDLEASAGDSSYGQDSSDYLPEGATDGSAQPILPAGGGPGGNPRLYDELIRVSVTIKNTGKVAGDEVPQLYVSLGGPNEPKIVLRQFERITLQPSEETTWNTTLTRRDLANWNVEKQDWEITSYPKMVFVGSSSRKLPLRASLPTVH

SEQ ID NO: 113 - protein, 838 aa, Aspergillus luchuensis
MRFTLIEAVALTAVSLASADELAYSPPYYPSPWANGQGDWAQAYQRAVDIVSQMTLAEKVNLTTGTGYVLVRLAGFPADYNSAFPSGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNHYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASNYLYPDGLQKITKFIYPWLNSTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 114 - protein, 860 aa, Aspergillus heteromorphus
MRFGWIEAAALTAASVVSADELAYSPPFYPSPWANGQGDWASAYERAVAIVSQMTLAEKVNLTTGTGWELEKCVGQTGGVPRLGIPGMCGQDSPLGVRDSDYNSAFPAGINIGATWDKNLAYLRGQAMGQEFSDKGADFQLGPVAGPLGRAPDGGRNWEGFSPDPALTGVLFAETIKGIQDAGVVATAKHFIGYEQEHFREAPEAQGYGYNITESGSANIDDKTMHELYLWPFADAVRAGVGAIMCSYNQINNSYACQNSYALNKLLKGELGFQGFVMSDWEAHHSGASAAMAGLDMSMPGDVVFDSGTSYWGTNLTIGVLNGTVPQWRVDDMAVRIMAAYYKVGRDRLWTPPNFSSWTRDEYGFKYYYSSEGPYEVVNHFVDVRRNHSDLIRRIGADSTVLLKNDGCALPLTGTERKVALIGEDAGSNPYGADGCSDRGCDNGTLAMGWGSGTTEYPYLVTPEQAIQAEVIQNGGTVFAVTDNWAISQMETLAAEATVSLVFVNADSGEGYIDVDGNMGDRNNLTLWGNGDNVIKAAASNCNNTIVIIHSVGPVLVNEWYDHPNVTAILWAGLPGQESGNSLADVLYGRVNPGAKSPFTWGKTRESYQDYLITEPNNGDGAPQEDFTEGVFIDYRGFDKRNETPIYEFGYGLSYTTFNYSQLQVQALNASAYTPASGETEAAPTLGEAGNASDYLYPSGLQRITAFIYPWLNSTDLEASSGDSSYGQSSDFLPEGATDGSAQPILPAGGGPGGNPALYDDLIQVSVTIKNTGDIAGDEIPQLYVSLGGPDEPRIVLRKFDRITLQPSEEYEWTTTLTRRDLSNWDVAAQDWIVTSYPKKVYVGSSSRKLPLRASLPTVQ

SEQ ID NO: 115 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 116 - protein, 861 aa, Aspergillus caelatus
MKLGWFEVAALAAASVVSAQDDLAYSPPFYPSPWADGQGEWAEVYKRAVEIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYILNEQEHFRQQPEAAGYGFNVSDSLSSNVDDKTIHELYLWPFADAVRAGVGAVMCSYNQINNSYGCENSETLNKLLKAELGFQGFVMSDWTAHHSGVGAALAGMDMSMPGDVTFDSGTSFWGANLTIGVLNGTIPQWRVDDMAVRIMAAYYKVGRDTKYTPPNFSSWTRDEYGFAHNHVSEGAYERVNEFVDVQRDHADLIRRIGAESTVLLKNKGALPLSRKEKLVALLGEDAGSNPWGANGCDDRGCDNGTLAMAWGSGTANFPYLVTPEQAIQNEVLQGRGNVFAVTDSWALDKIAAAARQASVSLVFVNSDSGEGFLSVDGNEGDRNNITLWKNGDNVVKTAANNCNNTVVIIHSVGPVLINEWYDHPNVTGILWAGLPGQESGNSIADVLYGRVNPGAKSPFTWGKTRESYGSPLVNEANNGNGAPQSDFTQGVFIDYRHFDKFNETPIYEFGYGLSYTTFELSDLHVQPLNASQYTPTSGMTEAAKNFGETGDASEYVYPEGLERIHEFIYPWINSTDLKASSDDSNYGWEDSKYIPEGATDGSAQPRLPASGGAGGNPGLYEDLFRVSVKVKNTGKVAGDEVPQLYVSLGGPNEPKVVLRKFERIHLAPSQEVVWTTTLTRRDLANWDVSAQDWTVTPYPKTIYVGNSSRKLPLQASLPKAR

SEQ ID NO: 117 - protein, 854 aa, Madurella mycetomatis
MANLVLALLLFLSALTSAATDTPFLGYPSPWATNITDPTWAAAYAQAIAFVANLTLTEKVNLTTGTGWEADRCIGATGGIPRLGFRPFCLMDGPLGVRYTDHNSAFPAGVNTAATFSRRLMRLRGEAMGAEFRGKGIDVMLGPVAGALGRVPQGGRNWEGFSPDPYLTGVAMAETIQGIQSRGVVACAKHYILNEQEHFRGSIDVRIDDRTMHELYLWPFADAVRAGVGSVMCSYNKINGTYACENEWTTNYLLKNELGFQGFVLSDWGAQHNTLGSALGGLDMAMPGDGGPPPYRAWWGGALTEAVLRGDVPQWRLDDMAVRIMAAYFRVHTGNYTSRPDINFSAWTNSTVGPLYPAANQSYTVVNEFVDVQSDHASLIREIGAKSVVLLKNAYDLLPLRQPGPPIIAVIGDDAQDHPLGPNACPERGCLNGTLAMGYGSGTANFPYLVSPLTALTEQARADNTTLLYAPSNWDLDAAITTARNASIAIVFAAATSGENFITVDGNAGDRNNLTLWANGDALIKAVASINPNTIVVLHTPGPVILDYAEEHPNISAILWAGLPGQESGNALVDVLYGKVNPQGRSPFTWGDSVEEYGAQLMFEAENPRAPVQSFDEGVFIDYRKFSTFGGKLTYPFGFGLSYTKFRYSGLSVVRKEEVEGRFEPAEGLTGPAPTFGVVEAELGVHTAPEGFTRISPYVYPWLNNSESLVTGNSTGASEFPAAARNGSAQPVLPASGAPGGNPGLYEVLYTITASIENVGEVAGTEIPQLYVQLGGEENPFGVLRGFDEVELEPGETKNVTFELTRRDVSNWNTSTQNWEITDREKVVFVGSSVRDIRLNASLPAPVGMAWRHG

SEQ ID NO: 118 - protein, 975 aa, Aureobasidium pullulans
MSQSQDYELTPRESPRQSLDHPEDYRLSTSSRDSISSSINELDPLKTPNRPYKDDVSQTHDSVFVTRRRSTWRRYLLPSRMCCMMVLLFTAALVMLLSAGGIWVYKTEPEGGQSDPWYPAPRGGSVTAWEEAYKKAAALVGQMSVVEKVNITTGTGWEMGMCVGNTGPVERLGFPSLCLQDGPLGLRFADNITAFPAGITLGTTWNKELIYLKGRAHGREARGKGVHIALGPSMGPFGRLPAGGRNWEGFGSDPVLQGLAAAAMIRGIQEEGIIATAKHYIANEQEHFRQSWEWGTPNAISSNLDDRTLHEIYAWPFAESVKAGVGSVMCSYNLVNQSYACQNSKLLNGILKDELGFQGFIQSDWLAQRSGVASALAGLDMSMPGDGLRWMDGQSVWGGELTKAVFNGSVPMERLNDMVLRVVAAWYQFGQDDKKKWPAEKDGGGPNFSSWTNDEIGLLHPGSNDTTKGVVNKFVNVQGEGDDSHGKLARQIAAEGTVLLKNDDHVLPLNRDGKKLTKNGDKLRVAIFGEDAYNNGKGPNACADRACNEGTLAQGWGSGTVEFPYLVAPAEGIHLGFDVESVTLSDYPSNYEDLDKHPSRAADQDLCFVFINSDGGEGYVAWKDVKGDRNDLYAQKGGDELVQKVAAKCGGPTIVVVHAVGPVILEKWIDLPGVKAVLYANLPGQESGNALASIVFGDINPSGRLPYTIAKNEDDYGPTSKILYRPNAVIPQQNFSEGLYVDYRYFDKHDIEPRYEFGFGLSYTTFHLSSLFIHPLATEYSTLPAKRPESITPPHLDVSIPDAREALWPSNFIALKKYIYPYINSVDDIKTGKYPYPVGYEIAQPLSQAGGAEGGNPDLYTPLAVVQASLTNTGSLPGDCVVQLYISYPSTPIIDSLGNTVDFPVRVLRAFDKLFVTPEKRVEVSLQLTRKDLSFWDVGLQNWVLPMGDFEVQLGFSSRDIQQKGDILSFSRLLL

SEQ ID NO: 119 - protein, 1220 aa, Fusarium oxysporum
MALIICFYYPISLGEAPDSDHTLKEKILGLGLKSAVILAGALVCLFLALQWGGTKHPWSDSRVWGCLVGFGLLLTLFVYIQIRQGEAALIRPRIISQRSVFLGCLFSALYQGAMTTQSYHLPFYFQAVKGVDPQSSGVDILPHGVTVTIATLITGSIITWLGYYVPFMWAGSAIFTIGAGLLYTISQNTPLARWFGYEVLAGAGFGIAIQIPIFAVQVVLVAGDIPLGTVLIILSQALGGSVGLSISQNVFQNSLRQRLNTIADIDIQVVIAAGGTDLEHVVSADSLAHVRDAFRYGISNAFLVSTALGGVAVLASIGMERKKIKSKKEGVDLYKTARPFDVSVFSSRGSIDTKMTLAKVAFTLANLVAVGSSAKNVADGFVAAPYYPAPYGGWDEAWADSYARAKKMVDSMTLAEKTNITAGTGLFMADRVGFPQLCLNDAANGVRLADNVTVFPDGITAGATFDKKLMYERGVAIGKEARGKGVNVWLGPSVGPIGRKPKGGRNWEGFGADPSLQGIGARETIKGVQEQGVIATVKHLIGNEQEMYRRQTTDVVSPAYSANIDDRTMHELYLWPFAEAVKVGVGATMTAYNRVNGTISSEHSYLINALLKEELGFQGFVMTDWLSQITGVQSAIAGMDMSMPGDTIIPLFGNSLWMYELTRSALNGSVPMSRLNDMATRIVATWYQFEQDKDFPSVNFDTNTYNRVGPLYPAAWPNSPSGVVNQYVQVQDDHDEIARQIAQDAITLLKNDEKLLPLSTKSSLKVFGTGAQTNPDGANACVDRSCNKGTLGQGWGSGTVDYMYLDDPIGAIKKAAGDVTFYNTDKFPSVPSPSDDDVAIVFVTSDSGENQYTVEGNNGDRNADKLNVWHNGDALIKAAAAKYKNVVVVIHTVGPVLVDQWIDLPSVKSVLVAHLPGQEAGKSLTNILFGDASPCGHLPYSITKKEDDMPESVTKLIDSGFIDAPQDTYSEGLFIDYRWLNKEKIQPRYAFGHGLSYTNFTYTNATIKRGTQLSQYPPKRPAKGKVLDYSQDIPDYKEAIKPSSFKTVWRYLYSWLSESDAKSAAAKAETSKYPYPDGYSTAQKTALPKAGGVSGGNPALWDEAYTLSVVVTNAGSKFSGKASVQAYVQFPSVAGYETPVIQLRDFEKTKVLEPGSSETVQLTLTRKDLSVWDVKAQDWLVLDGEFKIWLGSASDKLDAVCFTDDLGCEHDVKGPVSYDS

SEQ ID NO: 120 - DNA, 1437 bp, Cellulomonas wangleii
  source 1..1437: mol_type = genomic DNA
atgacccccccaccccagctccgacgcgggcgccccctggtcgcacgcggcgcctccgccatcgcgatcgtcgcgctcgcggtgacggccgccgcggccatcccggcgcaggccgccggctcgacgctccaggaggccgccgcccagagcggccgctacttcggcaccgcgatcgccgcgaacaagctcagcgactcgacctacgtgaccatcgcgaaccgtgagttcaacatgatcacggccgagaacgagatgaagatggacgcgacggagccgaaccagaaccagttcaactacagccagggcgaccggatcctcaactgggccaagcagaacggcaagcaggtccgcgggcacgcgctcgcgtggtactcccagcagccggagtggatgaagcgcatggagggctccgcgctgcgcagcgcgatggtcaaccacgtcacccaggtggcgacgcactacaagggtcagatctacgcctgggacgtcgtgaacgaggcgttcgccgacggcagctccggcggacgccgcgactccaacctggagcgcaccggcagcgactggatcgaggtggccttccgcgccgcgcgcgccgccgacccggccgcgatcctctgctacaacgactacaacatcgacgactggtcgcacgccaagacccagggcgtctaccggatggtccgtgacttcaagcagcgcggcgtcccgatcgactgcgtcggcctgcagtcgcacttcaacccgcagagcccggtcccggccaactaccagacgacgatcgagagcttcgccgccctcggcgtcgacgtgcagatcaccgagctcgacatcgagggctccggctcggcgcaggccgagaacttccgcaaggtgacccaggcctgcctcaacgtcgcccgctgcaccggcatcacggtgtggggcgtgcgtgacacggactcgtggcgcgcctcgggcaccccgctgctgttcgacggcaacggcaacaagaagcaggcgtacacctcggtgctcaacacgctgaacgcggcgagcccgaccacgccgccgcccaccacgccgccgccgacgaccccgccgccggtgacgcccccgcccacgaccccgccgcccaccacgccgccgccgacgaccccgccgccgacgaccccgccgccggtgacgggcagctgctccgccgcactgaccatcgccaacgcgtggcccggtggctaccagggcacggtcacggtgacggccggctcgtcgtccctcaacgggtggcgggtgaccctgccgggtggcgtctccacgaacaacgtgtggaacggcgtggtctccggcaacgtcgtgagcaacgcgccctacaacggctccgtcggggccgggcagtcgacgaccttcgggttcatcgggaacggcaacgccccgtcgccgacctccctcacctgctcctga

SEQ ID NO: 121 - DNA, 1419 bp, Cellulomonas shaoxiangyii
  source 1..1419: mol_type = genomic DNA
atgctccgacgagggagcaggcgcggccccggcgctcgaggcgcagccgtcctcgccgccctgaccctcgcggcgaccgccgcggccgccatcccggctcaggccgccggctcgacgctgcaggacgcggccaacgaccgcggccgctacttcggcacggcgatcgccgcgaaccgcctcagcgactcgacctactcgaccatcgcgaaccgtgagttcgacatgatcacggccgagaacgagatgaagatggatgcgacggagccgtcgcagaaccagttcaactacaccaacggcgaccgcatcgtgaactgggcgctccagaacggcaagcaggtccgcgggcacgcgctggcgtggcactcgcagcagccgggctggatgcagaacatgtccggcacggcgctgcgcaacgccatgctcaaccacgtcacgcaggtcgccacgcactaccgcggcaagatctacgcctgggacgtcgtgaacgaggccttcgccgacggctcgtccggcgcccgccgcgactccaacctgcagcgcaccggcaacgactggatcgaggcggccttccgcgccgcccgggccgccgaccccggcgcgaagctctgctacaacgactacaacaccgacgactggacgcacgcgaagacgcaggccgtgtacaacatggtccgcgacttcaaggcgcgcggcgtcccgatcgactgcgtcggcctgcagtcgcacttcaacgcgcagagcccggtgccgagcaactaccagacgaccctgtcgagcttcgccgcgctcggcgtggacgtgcagatcaccgagctcgacatcgagggctcgggctccgcgcaggcggagagctaccggcgcgtcgtccaggcgtgcctcaacgtctcccgctgcaccggcatcacggtgtggggcgtgcgggacaccgactcgtggcgcgcctcgggcacgccgctgctcttcgacggccagggcaacaagaaggcggcgtacaccgcggtcctcgacacgctgaacagcggacccgggaccaacccgacgacgcctccgcccaccagccccccgccgacgaccccgccgccggtcaccccgccgccgacgacgcccccgcccacgaccccgccgccggtcaccccgccgcccagcggcacctgctccgcggcactgacgatcgcgaacagctggggcggcggctaccaggccaccgtcacggtgcgggccggttcgtccgctctcaacggatggcgggtcaccctgcccggcggcgtcacgacgtcgaacctgtggaacggcgtcctcgccggcagcgtcgtgacgaacgcgccgtacaacggctccgtcggagccggccagtcgacgtccttcgggttcatcggcaacggcagcgcacccgccgcgggagcgctgagctgcagctga

SEQ ID NO: 122 - DNA, 1446 bp, Micromonospora sp. WMMA1998
  source 1..1446: mol_type = genomic DNA
atggacaaggtgctcgcccgcggcagcgggagcccgaccgcccgataccggccgcgtgccgccctgctgtcggccgccgtcggcgcggcgctggtcgcggccacggtcgcgatggcgaccagcgccagcgccgggaccaccctgggcgcggcggcggcggaacagggccgctacttcggcaccgcggtggccgcgaacaagctgtcggacgccacgtacgtcggcatcctgaaccgcgagttcactatggtcacccccgagaacgagatgaagtgggacgccaccgagccgtcccagggccagttcagctacaccaacgccgaccggatcgtcgcccacgcccaggccaacggcatgcgggtacgcggccacgcgctggcctggcactcgcaacagcccggctgggcgcagaacctgtccggcagcgcgctgcgccaggcgatggtcaaccacatcacccaggtcgccacccactacaagggcaagatctacgcctgggacgtggtcaacgaggcgttcgacgacggcagcggcggccggcgcgactccaacctccagcgcaccggcaacgactggatcgaggtggcgttccgcaccgcgcgcgccgccgacccgggcgccaagctctgctacaacgactacaacaccgacaactggacctgggccaagacccagggcgtctacaacctggtcaaggacttcaaggcccggggcgtgccgatcgactgcgtcggcctgcagtcgcacttcaacagcggctcgccgtacccgagcaactaccgcaccacgttgcagaacttcgccgccctcggcgtcgacgtgcagatcaccgaactcgacatcgagggttcgggcagcgcccaggccaccacgtacgccaacgtggtcaaggactgcctggccgtgtcgcggtgcaccggcatcaccgtctggggaatccgggacagcgactcctggcgggccggcggcaccccgctgctcttcgacggcaacggcaacaagaaggcggcctacaccgccgcgctcgacgcgctcaacgccggcggcacgccgccgccgaccaccacgccgccgaccaccacgcccccgaccacgactccgccgacgaccacgccgcccaccaccgcgccgcccacgactcccccgcccaccggcggcgggtgcaccgcgtcgctcaccaccaaccagtggcccggcgggttcgtcaccaccgtgcgcgtcaccgccggcgccggcgcgctcaacggctggacggtgacgctcaccgtgccgtccggctcggcggtcaccaacacgtggagcgcccaggccagcggcgccagtggcgcggtgacgttccgcaacgtcgcctacaacggccaggtcggtgccggcggcagcaccgagttcggcttccaggggaccggcacggccccctccggcacgcccacctgcgcggcgggctga

SEQ ID NO: 123 - DNA, 1425 bp, Xylanimonas cellulosilytica
  source 1..1425: mol_type = genomic DNA
atgaaccatcagcaccaccgtcgtaggttgcgcagcgtcgcagccgtcgcgctcgcctccctggtcgtcgccacgggagtggtgaccgcccaggcggccggatccacgctccaggaagcggcgggcagccgctacttcggcacggccatcgcggccaacaagctctcggactcgacctactcgaccatcgcgaaccgtgagttcgacatgatcacggccgagaacgagatgaagatggacgccaccgagccgtctcagggcagcttcaacttcaccaacgccgacaggatcgtcgactgggccaccgccaacggcaagcggatgcgcggtcacgccctcgcgtggcactcgcagcagccggggtggatgcagaacatgtcgggcaccgcgctgcgcacggcgatgctcaaccacgtcaccgaggtcgctgcgcactacaagggcaagatctacgcctgggacgtcgtcaacgaggccttcgccgacgggtcctcgggcgcccgccgcgactcgaacctgcagcgcacgggcgacgactggatcgaggccgcgttcagggccgcccgggccgccgacccgtccgccaagctctgttacaacgactacaacacggacaactggaactgggagaagacccaggccgtgtacgccatggtcaaggacttcaaggaacgcggcgtgccgatcgactgcgtcgggctccagtcccacttcaactcgggcagcccctacccgagcaactaccgcacgacgctgcagaacttcgcggcgctcggcgtcgacgtccagatcaccgagctggacatcgagggctcgggcagcacgcaggccgacacgtacgccaaggtggtcgccgactgcctcgcggtgagccgctgcaccggcatcacggtgtggggcgtgcgcgactccgactcgtggcgcgcgagcggtacgccgctgctgttcgacgcgtcgggcaacaagaaggccgcctacacgtcggtgctgaacaccctcaacggcggtgcgacgccgacgccgacgccgacgccgacgccgacgccgaccccgactcccacgccgactccgaccccgacgccgacgccgactcccacgccgacgccgacgccgacgccgactcccacgccgacgccgacgtccggtccgtgcaccgccacgatgacgatcacgaactcgtggcagggcggcttccagggtgaggtcacggtcaaggcgggcagtgcccgcacgtcctggtcgacgtcgttcacctcggccgccacggtccaagtctggaacggggtgcactcgacctccggctcggtgcacacggtgtcgaaccagccgtacaacggcacgctggccgccggtgcgtcgacgacgtacggcttcacggccaccgggacggcgccgagcagcccgcccgcggtgacctgctcgtga

SEQ ID NO: 124 - DNA, 1443 bp, Verrucosispora sp. WMMD573
  source 1..1443: mol_type = genomic DNA
atgaacaacgcccacgcccgcgccagcgggcggccggctacccgactgcgaccgcgcgccgcgctggtctcggccgcggtcggtctcgccctcgtcgccaccagcatggtggtgacctccagcgcccaggccgccgagtccaccctcggtgcggccgcggcccagtccggccggtacttcggtgccgcggtggcggcgaacaagctgtccgactccacctacgtcggcatcctgaaccgcgagttcaactcggtcacggccgagaacgagatgaagatcgacgcgctcgagccgcagcagaacaacttcacgttcggcaacgcggaccggatcgtcaaccacgccctgtcccggggctggaaggtccgggggcacaccctggcctggcactcgcagcagcctggctggatgcagcagatggaaggcacggcgctgcgtaacgcgatgctgaaccacgtcacccgggtcgcgacctactaccgcggcaagatcgactcgtgggacgtggtgaacgaggcgttcgacgacggcaacaacggcgcgcgccgtaactcgaacctgcagcgcaccggtaacgactggatcgaggccgcgttccgggccgcccgggccgccgacccgggcgccaagctctgctacaacgactacaacaccgacaactggacctgggccaagactcaggccgtctaccggatggtgcaggacttcaagcagcgtggcgtgccgatcgactgcgtcggcctccagtcgcacttcaacagcggctcgccgtatccgagcaactaccgcaccacgctgtccagcttcgccgcgctcggcgtggacgtgcagatcaccgagctggacatcgaaggctccggcagcacccaggccaacacctaccgcaacgtggtcaacgactgcctcgcggtgccccgctgcaacggcatcaccgtgtggggaatccgggacaccgactcctggcgttccggtggcaccccgctgctcttcgacggcaacggcaacaagaagcaggcgtacgacgcgacgctccaggcgctgaactcgggcggcaccaccccgccgcccacgaccccgccgccgcccacgaccccgccgcccaccacgccgccgcccacgaccccgccgccgaccaccccgccgcccggcgggtccggctgcacggcaacggtgtccctgaaccagtggaacggtggtttcgtcgccacgatccgcgtcaccgccggttccgcgagactgaacggctggtcggtgggcatcgggattccgggcggtagctcggtgaccaacacctggaacgcccaggccagcggcaacagcggcaacgtgacgttccgcaacgtcagctacaacggcaccgtgaacgccggtgccaccacggagttcggcttccagggcaccggtaccggtccatccggtacccccacctgcaccggtagctga

SEQ ID NO: 125 - DNA, 2043 bp, Saccharothrix syringae
  source 1..2043: mol_type = genomic DNA
atgtccagaactgctgtcacagctgccggcaggtcggaggcacgaggccggtcgcggcgcgccgcggtggtggtcggtgcgatcgggttgctgagcgcggcggccgtggtgctgccgaacgtggccaccgccggcaccacgctgggcgcgtccgccgcggagagcgggcgctacttcggcacggcggtcgcggccaacaagctctcggactcgacctacgtgggcatcctcaaccgtgagttcgacatggtcacggctgagaacgagatgaagatggacgccacggagccgaaccagaaccagttctcgttcggcaacggtgaccggatcgtcaaccacgcccgcaaccagggcaagcgggtccgtggccacgcgttggcgtggcactcgcagcagccgggctggatgcagaacatgtccggcacggcgttgcgcaacgcgatgctcaaccacgtcacccaggtcgccacgtactacaagggcaagatctacgcctgggacgtggtcaacgaggcgtacgccgacggcagctccggcggtcggcgtgactcgaacctccagcggaccggcaacgactggatcgaggccgccttccgggccgcccgcgccgccgacccgaacgcgaagctgtgctacaacgactacaacaccgacaactggtcgcacgccaagacccagggcgtgtaccggatggtgcaggacttcaagtcgcgcggtgtgccgatcgactgcgtcggcttccaggcgcacttcaacagcggcaacccggtgccgtcgaactaccacaccaccctgcagaacttcgccgacctcggcgtggacgtccagatcaccgagctggacatcgagggctccggcagcacccaggcccagcagtaccagggcgtggtccaggcgtgcctcgcggtgacccgctgcaccggcatcacggtgtggggcatccgcgacagcgactcgtggcgttcctcgggcaccccgctgctgttcgacggctcgggcaacaagaaggccgcctacacctcggtcctcaacgccctcaacgcgggcagcaccgcgccgccgtccaccaccacgacgacgaccccgccgaacacctcggactgccgcgccggctacgtcggcctgacctacgacgacggccccaacggcagcaccaccacgcagctgctcaacgcgctgcggtcggccggcctgcgcgccacgttcttcaaccagggcaaccgggtccagcagaaccccggcctggccaaggcgcagcgcgacgccggcatgtgggtcggcaaccacagctggagccacccgcacatgacccagctgagccagtcgcagatggcctcggagatctcccagacccagcaggccatccagtcggccaccggtgaggcgccgaagctgttccgcccgccctacggcgagaccaacagcacgctcaagtcggtcgaggcccagtacggcctgaccgaggtgctgtggagcgtcgactcgcaggactggaacaacgccagcaccgcgcagatcgtgcaggccgcgtcgaccctgcagaacggcggcgtgatcctgatgcacgacggctaccagacgacgatcaacgccatcccccagatcgcggccaacctcgccagccgcggcctgtgcgccggcatgatctccacgtcgaccggccaggccgtcgcgccgaacgacaacccgcccaccaccggcccgacgaccaccaccacgacgacgtcccagcagcccggcggctcgtgcaccgcgacctaccggaccacccagcagtggggcgaccgcttcaacggcgaggtgaccgtccgggcgggcgcctccgcgatcaccagctggacggccaccgtcacggtgacctccccgcagaaggtgtcggccacctggaacggcacgccgagctgggactccagcggcaacgtcatgaccatgaagcccaacggcaacggaaacctcgccgcgggcgcgagcacgacgttcggcttcaccgtgatgaccaacggccagtgggcggcgccgaccgtctcctgccgcacgccgtga

SEQ ID NO: 126 - DNA, 1410 bp, Cellulomonas palmilytica
  source 1..1410: mol_type = genomic DNA
atgaccccctcatccatgtccagacgcgcgcgcttcgcggccgcgctcgcagtcgtcacgctcggggcgaccatcgctgcgacgatccccgcccaggccgcgggaagcacgctgaaggatgccgcagcgcagagcggccggtacttcggcaccgcgatcgccggcttcaagctgagcgactcgacgtactcgtccatcgcgaaccgtgagttcaacatgatcacggccgagaacgagatgaagatggacgcgacggagccgtcgcagaacaacttcaacttctccagcggcgaccagatcctcaactgggccgtccagaacggcaagcgcgtgcgcggccacgccctggcctggcactcgcagcagcccagctggatgcagggcatgtccggcagcgcgctgcgcagcgccatgctcaaccacgtcaccaaggtcgccgagcactacaagggcaaggtctatgcctgggacgtcgtgaacgaggcgttcgacgacagcaacggcgggcgccgcgactccaacctgcagcgcaccggcaacgactggatcgaggccgcgttcaaggccgcccgcgccgccgacccgaacgccaagctctgctacaacgactacaacaccgacaactggacctgggccaagacgcagggcgtctacaacatggtcaaggacttcaaggcccgtggcgtgccgatcgactgcgtcggcttccagtcgcacttcaacgcgcagagcgcctacaacagcaactaccgcaccacgctgtcgagcttcgcggcgctgggtgtcgaggtccagatcaccgagctcgacatcgagggctccggctcgcagcaggcggacacgtaccgtcgcgtcgtcgaggactgcctcgccgtcaaggcctgcaccggcatcacggtgtggggcgttcgtgactccgactcgtggcgctcctacggcacgccgctgctgttcgacaacaacggcggcaagaaggccgcgtacacctcggtcctcaacgcgctgaacgccgcggacccgacggacccgacgaccgaccccacgcaggacccgacggacgacccgacccaggacccgacggacgacccgacggacgacccgacggacccggagacgacgaacccgccggacccgaccggtaagtgctccgcggcgctcacgatcgtgaactcctggccgggcggctaccaggccacggtgacggtcaaggccggctcctcgtcgatcaacggctggcgggtcaccctgcccagcagtgtgaacacgaacaacctgtggaacggcgtcctgtccggtggcgtggtgaccaacgccccgtacaacggctcggtcggtgccggccagtcgacgaccttcgggttcgtcggcaacggcagcgccccgggcgcaggcaacctgacctgcgcctga

SEQ ID NO: 127 - DNA, 1374 bp, Micromonospora carbonacea
  source 1..1374: mol_type = genomic DNA
atgagacgcaagagagccctcctgacgacggtgaccctcgcggtcaccggcgcactcaccgccggcgtgctggtgacgatggcccccgccgccagcgccgggacgaccctgcgggctgccgcggccgagaagggccgctacttcggcgccgcggtcgcgacgggcaaactctccaacagcacgtacacgacgattctcaaccgcgagttcaacagcgtcgtggccgagaacgagatgaagtgggacgccaccgagccgcagcagggccagttcaactacagcggcggcgaccgcctcgtcagccacgcccgggccaacgggatgagcgtgcggggccacgccctgctctggcaccagcaggagccgggctgggcgcagggcatgtccggcagcgccctgcgcagcgcgatgatcaaccacgtcacccaggtcgccacccacttcaaggggcagatctacgcctgggacgtggtgaacgaggcgttcgccgacggcaacagcggcggccggcgtgactcgaacctccagcgcaccggcaacgactggatcgaggcggcgttccgcgccgcgcgggccgccgacccgggcgcgaagctctgctacaacgactacaacaccgacggggtcaacgcgaagtcgaccggcatctacaacatggtgcgcgacttcaagtcccggggcgtgccgatcgactgcgtcggcttccagtcccacctgggcaccacgctggccagcgactaccaggccaacctccagcgcttcgccgacctcggcgtcgacgtgcagatcaccgagctggacgtcatgaccggcggcaaccaggcgaacatcttcggcgcggtgacccgggcgtgcatgaacgtgtcgcgctgcaccggcatcacggtgtggggcgtgcgggactgcgactcgtggcgggggtccgacaacgccctgctgttcgactgcaacggcaacaagaagccggcgtacgactccgtcctcaacgccctcaatgccggcaccggcatccccaacccgacgaccaccccgccgaaccccaccaccactccgccgaacccgacgactccgccgccgggtggggccgggtgttcggcgacggtgtcggcgaattcgtggacgggtggtttcgtggccacggtgaaggtgaccgctggttctggtggtacccggggttggaacgtgagtgtgacgttgccgggtggtacgagtgtcacgggtacgtggtcggcgacggccagtggtagttcggggacggtgcggttcgccaacgtggactacaacggtcagctcgccgccggtcaggtgaccgagttcgggttccagggcaacggcaccgcgcccacccagacccccacctgcaccgccagctga

SEQ ID NO: 128 - protein, 469 aa, Cellulomonas palmilytica
MTPSSMSRRARFAAALAVVTLGATIAATIPAQAAGSTLKDAAAQSGRYFGTAIAGFKLSDSTYSSIANREFNMITAENEMKMDATEPSQNNFNFSSGDQILNWAVQNGKRVRGHALAWHSQQPSWMQGMSGSALRSAMLNHVTKVAEHYKGKVYAWDVVNEAFDDSNGGRRDSNLQRTGNDWIEAAFKAARAADPNAKLCYNDYNTDNWTWAKTQGVYNMVKDFKARGVPIDCVGFQSHFNAQSAYNSNYRTTLSSFAALGVEVQITELDIEGSGSQQADTYRRVVEDCLAVKACTGITVWGVRDSDSWRSYGTPLLFDNNGGKKAAYTSVLNALNAADPTDPTTDPTQDPTDDPTQDPTDDPTDDPTDPETTNPPDPTGKCSAALTIVNSWPGGYQATVTVKAGSSSINGWRVTLPSSVNTNNLWNGVLSGGVVTNAPYNGSVGAGQSTTFGFVGNGSAPGAGNLTCA

SEQ ID NO: 129 - protein, 437 aa, Promicromonospora iranensis
MTRTLTSPRHRRSLRAISLATLTAVVLAGGMAATTAQAAGSTLQAAATEKGRYFGTAIAANKLSDSTYTTIANREFNMVTAENEMKIDALEPNQNQFNWTNGDRIVSWARSNGKQVRGHTLAWHSQQPGWMQNMSGTALRNAMLNHVTQVATHYRGQIHSWDVVNEAFQDGSSGARRDSNLQRTGNDWIEAAFRAARAADPNAKLCYNDYNTDDWTHAKTQAVYNLVRDFKARGVPIDCVGLQSHFNAQSPVPSNYQTTISSFAALGVDVQITELDIEGSGSAQADNYRKVVQACLAVSRCTGISVWGVRDTDSWRASGTPLLFDGNGNKKPAYTATLDTLNGGTSNPQPGGCTVAVTRSTDWSDRFNVSLAVSGSSTWRVSIQLQGGQTLQNSWNATVSGSSGTLTATPNGAGNNFGITVYKNGNTNLPTATCSTT

SEQ ID NO: 130 - protein, 480 aa, Micromonospora ferruginea
MDKVFARTSHSTAARPRTRAAVVSMLAGAAAVAATVAVATSASAGTTLGASAAEQGRYFGTAVAANKLSDATYVGILNREFNMVTPENEMKWDATEPSQNQFTYSSSDRIVAHAQANGMRVRGHALAWHSQQPGWAQSLSGTALRNAMVNHITQVATHFKGKIYAWDVVNEAFDDGNGGRRDSNLQRTGNDWIEVAFRTARAADPGAKLCYNDYNTDNWTWAKTQGVYNMVKDFKARGVPIDCVGFQSHFNSGSPYPGNYRTTLQNFADLGVDVQITELDIEGSGSAQATTYGNVVKDCLAVARCNGITVWGIRDSDSWRASGTPLLFDGSGNKKAAYTSTLNALNAGGTTPPTSTPPTSTPPTSTPPTTPPPTTTPPTTPPPAGACTASLTTNQWQGGFVTTVRVTAGGSALNGWSVSLTLPSGSSVTNTWSAQASGAGGAVTFRNVDYNRQVGAGGSTEFGFQGTGTAPSGTVSCTAG

SEQ ID NO: 131 - protein, 444 aa, Actinoplanes friuliensis
MLSAASAGVVLAASIAVVGTAEAASTLGASAAQTGRYYGAAIAAGRLGDSTYTRILNTEFNSVTPENEMKWDATEPSQGRFTYTNGDRILNQGLSNGSKVRGHALLWHAQQPGWAQALSGSALRNAAINHVTQVATHYKGKIYAWDVVNEAFADGGSGGRRDSNLQRTGNDWIEAAFRAARAADPAAKLCYNDYNTDGINAKSTGIFNMVRDFKSRGVPIDCVGFQSHLGTGIDSTYQANLKRFADLGVDVQITELDIEQGGNQANIYSTVTKACLAVSRCTGITVWGIRDTDSWRTGANPLLFDGSGNKKPAYTAVLNALNAGGTTNPTTPPVTSPPASPTTPPPSGSGCTATVSLNSWSGGYVATVKVTAGSSAVTGWTVSATLPSGGALTGVWSATNTGTTGAVSFRNVEYNGRIPAGGSTEFGFQGTGTGPSAAPACRVG

SEQ ID NO: 132 - protein, 759 aa, Actinoplanes ianthinogenes
MIVAGIAGLVAAGLGVVYTQTADAATTLSASANQTGRYFGTAIPVSKLGDSAYTTILKTEFNAVTPENEMKWDATEPSQGSFNYTNGDKILNQGRAQGAKVRGHALLWHSQQPTWAQSLSGSALRTAAINHVTQVATHYKGKIYAWDVVNEAFADGGSGGRRDSNLQRTGNDWIEAAFKAARAADPAAKLCYNDYNTDGVNAKSTGVYTMVKDFKARGVPIDCVGFQSHLGTTVPADYQANLQRFADLGVDVQITELDVAQGGNQANIYASVTKACLAVSRCTGITVWGIRDSDSWRTGENPLLFDNSGTKKAAYTSVLNALNAGGTKPATSATTTPPGTAAPSAGAGCTAKLTAGEVWGDRYNSTITVSGASTWTVVVTITAPQKVSTVWNGTATYGGGSGQIMTVKPNGNGNSFGFTTMNNGNSTARPTITSCVAGGSTTSATAAPTTVPASAPSSCALPSKYRWTSTGALATPKSGWASLKDFTVVPYNGKHLVYATTHDTGSSWGSMNFSPFTNFSDMASAGQNAMSSGAVAPTLFYFAPKKIWVLTYQWGGSAFSYRTSSDPTNANGWSAAKPLFTGSISGSGTGPIDQTIIGDGTNMYLFFAGDNGKIYRASMPIGNFPGNFGSSYTTIMSDTTNNLFEAPQVYKVQGQNQYLMIVEAIGSNGRYFRSFTATSLSGSWTPQAATESNPFAGKANSGATWTNDISHGELVRVTADQTMTVDPCHLQLLYQGRSPSSGGDYGLLPYRPGVLTLQR

SEQ ID NO: 133 - protein, 475 aa, Antribacter gilvus
MKDLSPHRAGKFPWRSLAGAAGALAVAASLAVGVSTSAQAAGTTLQAAAAESGRYFGTAIAANKLSDSTYTTIANREFNMITAENEMKLDATEPSQNQFNYTNGDRIVNWATSNGKQVRGHTLAWHSQQPGWMQNMSGTALRSAMLNHVTQVATHYRGKIHSWDVVNEAFQDGSSGARRDSNLQRTGNDWIEAAFRAARAADPNAKLCYNDYNTDDWTHAKTQAVYNLVRDFKARGVPIDCVGLQSHFNSQSPVPSNYQTTLSSFAALGVDVQITELDIEGSGTAQADNYRRVVQACLAVSRCTGITVWGVRDTDSWRSSGTPLLFDGSGNKKAAYTSTLNALNAGGTTNPPTTPPPTTPPPTTPPPTTAPPTTPPPTQPGACTAAYRLVNSWQGGFQAEVVVTAGSARTGWTTSFTLPGGTSISQLWSGTLTSSGSTHTVRNVSWNGNLGAGQSTTYGFTGTGSAPSSATVSCS

SEQ ID NO: 134 - protein, 653 aa, Nonomuraea jiangxiensis
MRTNALPPPRTRRSFGGFRRAVAVGVLALAGTIAPLALSVPADAAETTLGAAAMQSNRYFGAAIAAGKLNESAYTTIANREFNMVTPENEMKIDATEPNRGQFTFTNADRIYNWAVQNGKRVRGHTLAWHSQQPGWMQSLSGSTLRQAMIDHINGVMAHYRGNIYAWDVVNEAFADGNSGGRRDSNLQRTGNDWIEVAFRTARAADPAAKLCYNDYNIENWTWAKTQGVYNMVRDFKSRGVPIDCVGLQAHFNSGSPYNSNFRTTLSSFAALGVDVQITELDIQGASATTYANVTNDCLAVPRCTGITVWGVRDSDSWRSGDTPLLFDGSGNKKPAYTSVLNALNSVNPNPDTTPPSTPGTPAASNVTSSGATLTWAASTDTGGSGLAGYNVYREQGTTDPQLGQSATNSITLTGLTAGTQYQVYVRARDGAGNLSGNSQLVTFTTQTGGGTDTTPPSTPGTPAASNVTASGATLTWTASTDTGGSGLAGYDVYREQGATDPLLGQSATNSITLTGLTAGTQYQVYVRARDGAGNLSGNSQPATFTTTGGGTGGSCTVTPTTQTQWPSGYVIDPVRVTAGTSAISGWTVTFTLPAGHTVTGSWNTQLTVSGQTVTARNAAHNGNLGPGASTAFGFQVSRPNGNTSLPSGYTCA

SEQ ID NO: 135 - protein, 634 aa, Kibdelosporangium aridum
MSRTVGRHAALLALLGTFLLPGTADAGTTLGASAAEKGRYFGAAVAAHKLGDSTYVGILNREFNMVTPENEMKIDATEPNQNQFSFGNADRIVNHAVSQGMRVRGHTLAWHSQQPGWMQNMSGSALRQAMLNHVTRVASYYRGKIYAWDVVNEAFADGSSGARRDSNLQRTGNDWIEAAFRAARAADPNAKLCYNDYNTDDWSHAKTQAVYRMVQDFKSRGVPIDCVGLQSHFNNNSPYPSNYRTTLSSFAALGVDVQITELDIEGAPPTTYGNVVRDCLAVARCNGITVWGIRDSDSWRASQTPLLFNNSGGKKPAYDAVLSALNSGSIPPPGGSNCSAGYVGLTFDDGPNSSTTPQLLNALRSAGVRATFFNVGQRVQQNPALTRSQIDAGMWVGNHSWTHPHLTQMTAAQITSELSQTQQALQQATGQTPRLFRPPYGETNSTVRQVQAQLGLTEVMWTVDSQDWNNASTAQIVQAASTLQPGGIILMHDGYQTTVNAIPQIVANLSSRNLCAGMISTNGQVVAPGTEPGNGSCTASYRTTQQWGDRFNGEVTIKAGSAAITSWTSTVTITAPQRVSTTWNGTASWDSSGTVMTMKPNGNGNLAAGASTTFGFTVMANGQWAQPSVSCGSP

SEQ ID NO: 136 - protein, 472 aa, Actinomadura madurae
MRANVIPKSRIRIRKRAILAGALGVLATAALVAPSPAAAAESTLGAAAAQNGRYFGTAIASGKLNDSVYTTIANREFNSVTAENEMKIDATEPQRGRFDFSAGDRVYNWAVQNGKQVRGHTLAWHSQQPGWMQSLEGGTLRQAMIDHINGVMAHYTGKIVQWDVVNEAFADGSSGARRDSNLQRTGNDWIEVAFRTARAADPAAKLCYNDYNIENWTWAKTQAVYSMVRDFKQRGVPIDCVGFQSHFNSGSPYNGNFRTTLQSFAALGVDVAITELDIQGASATTYANVTNDCLAVSRCLGITVWGVRDTDSWRSEQTPLLFDGNGNKKAAYTAVLNALNGGTTTPPDGAGTIKGDGSGRCLDVPNASTTDGTQVQLWDCHGGTNQQWTYTDASELRVYGDKCLDAAGTGNGARVQIYSCWGGDNQKWRLNSDGSVVGVQSGLCLDAAGTANGSAIQLYSCWNGGNQRWTRT

SEQ ID NO: 137 - protein, 484 aa, Nonomuraea indica
MRVDAMSPPTARRPLGARRRTLITGVLGALGLITALAPAIPADAAASTLGAAAAQSGRYFGTAIASGKLGDSAYTTIAGREFDMVTAENEMKIDATEPNRGQFTFTAGDRVYNWAVQNGKRVRGHTLAWHNQQPGWMQSLSGSSLRQAMINHINGVMGHYKGKIYAWDVVNEAYADGNSGGRRDSNLQRTGNDWIEVAFRTARAADPAAKLCYNDYNIDNWTWAKTQGVYNMVRDFRARGVPIDCVGLQSHFNANSPYNSNYRTTIQSFAALGVDVQITELDIQGGSATTYANVVRDCLAVPRCTGISVWGVRDSDSWLGAGATPLLFDGNGNKKAAYTAVLDVLNGGSTTPPPGDAGQIRNTASGRCVDVPNSATADGTAVQLWDCNGQSNQRWTRTAAGELKYGDKCLDAGGTGNGARIQIYSCWGGDNQKWRLNSDGTIVGVQSGLCLDAVGGGTGNGTGLQLYGCWGGGNQRWDYNPGTA

SEQ ID NO: 138 - protein, 680 aa, Saccharothrix syringae
MSRTAVTAAGRSEARGRSRRAAVVVGAIGLLSAAAVVLPNVATAGTTLGASAAESGRYFGTAVAANKLSDSTYVGILNREFDMVTAENEMKMDATEPNQNQFSFGNGDRIVNHARNQGKRVRGHALAWHSQQPGWMQNMSGTALRNAMLNHVTQVATYYKGKIYAWDVVNEAYADGSSGGRRDSNLQRTGNDWIEAAFRAARAADPNAKLCYNDYNTDNWSHAKTQGVYRMVQDFKSRGVPIDCVGFQAHFNSGNPVPSNYHTTLQNFADLGVDVQITELDIEGSGSTQAQQYQGVVQACLAVTRCTGITVWGIRDSDSWRSSGTPLLFDGSGNKKAAYTSVLNALNAGSTAPPSTTTTTTPPNTSDCRAGYVGLTYDDGPNGSTTTQLLNALRSAGLRATFFNQGNRVQQNPGLAKAQRDAGMWVGNHSWSHPHMTQLSQSQMASEISQTQQAIQSATGEAPKLFRPPYGETNSTLKSVEAQYGLTEVLWSVDSQDWNNASTAQIVQAASTLQNGGVILMHDGYQTTINAIPQIAANLASRGLCAGMISTSTGQAVAPNDNPPTTGPTTTTTTTSQQPGGSCTATYRTTQQWGDRFNGEVTVRAGASAITSWTATVTVTSPQKVSATWNGTPSWDSSGNVMTMKPNGNGNLAAGASTTFGFTVMTNGQWAAPTVSCRTP

SEQ ID NO: 139 - protein, 471 aa, Cellulomonas cellasea
MNRSGLRPPSSTHRRRTGAVLVAALAVTLAGATLPAQGAGSTLQAAAAESGRYYGTAIAANKLSDSTYTTIANREFNMITAENEMKMDATEPSQNQFNYSSGDRIVSWARSNGKRVRGHALAWHSQQPGWMQNMSGTALRNAMLNHVTQVATHYKGQIYAWDVVNEAYADGSSGARRDSNLQRTGNDWIEAAFRAARAADPAAKLCYNDYNTDNWSHAKTQGVYTMVRDFKSRGVPIDCVGFQAHFNSGNPVPSNYDVTLRNFAALGVDVQITELDIEGSGSSQAQQYAGVHQACLSVARCTGVTVWGVRDTDSWRASGTPLLFDGSGNKKAAYTSTLNALNAGGTTTPNPTPNPTTPQPTPTTTTPPVTGTGSCTATYSEGQKWGDRFNGVVTIRANSAITSWTSTVTVSQAQRITSTWSGTPSWDSSGKVMTMRPAGNGTLAAGQTTSFGFTVLHGGDWTWPRVTCSAS

SEQ ID NO: 140 - protein, 457 aa, Couchioplanes caeruleus
MHKPRTPLRALGVAAAAAVVAAGSVVALTSTAEAATTLGASAAATGRYFGTAVAANKLSDSTYVGILDREFNAVTPENEMKWDATEPNQNQFNYSSADRIVSHAQAQNMRIRGHALAWHQQQPGWAQNLSGTALRNAMLNHVTTVAAHYKGQIYAWDVVNEAFDDGGNGARRDSNLQRTGNDWIEAAFRAARAADPGAKLCYNDYNTDGQNAKSNAVYAMVQDFKSRGVPIDCVGFQSHFNAQSPVPSDYQANLQRFANLGVDVQITELDIEGSGQTQADNFGRVVKACLAVSRCTGITVWGIRDSDSWRASGTPLLFDSSGNKKAAYTSTLNALNSGGTTPPSDPPTSPSTPPSTPTDPGTGACTATISLNQWNGGFVATVRVSAGSSALSGWSVSTTLPSGAAVTNSWSSQSSGSSGTVRFANVSYNGSVAAGGSTEFGFQGTGTGPSSATCSAS

SEQ ID NO: 141 - protein, 473 aa, Glycomyces tritici
MITSTPRRRRRVWSTIAAVATASLAATTALVMLPGTAQAGTTLGASAAESGRYFGAAVSTQYLSESAYANTLGAEFNSVVAENAMKWDATEPNPGQFNFSGGDQLVNWAQARGMKVRGHTLVWHAQQPGWAQNLTGQNLRNAMLNHIAGVANHYEGDVFAWDVVNEAFEWDGSRRQSNLQRQLGNGWIEEAFRAADAADPTATLCYNDYGTDSINAKSTAIYNMVRDFKSRGVPIDCVGFQTHIAHNENLSSYQANLQRFADLGVDVEITELDVGQGSGQAATYGTVTNACMAVARCKGITVWGITDKYSWRDDNPLLFDGNYQKKQAYHTVLAALNNGNPGGGGGKHITSAASGRCLDVPNQSTTNGTQLQIYDCWTGANQQWTVANGEISVYSGGSKKCLDASGGGTANGTAAIIWSCHGGANQRWNVNSNGTITNAASGLCLDVAAAATANGSKVQLWSCTGGSNQRWTV

SEQ ID NO: 142 - protein, 475 aa, Actinoplanes auranticolor
MTVIAARARLLLGAACAVVIAVSSVAVATLAHADLTGPPGTTLKAAAERSGRYFGAAMGRDRLTDNGFLTIANREFDMMTAVNEMKPDATEPNRGQFDFRAGDAIYNWATQRGMRFRGHTLAWHAQQPRFWGSLSGSALRQAMIDHINGVMAHYKGKLYAWDVVNEAFAENGSRRSSNLQATGNDWIEAAFRAARAADPGVQLCYNDYNIENWTYAKTQGVYNMIRDFKARGVPIDCVGLQTHFTGGSSLPGNFQQTLSSFAALGVDVALTEADVTNASSSQYQGLTQACMNVPRCVGITTWGIRDSDSWRGNENPLLFDRNSNPKPAYTSVLNALNAASTTVPGTSTPPSTPPSTPPSTPPSTPPSTPPVTGQPGACTATYRTASTWNGGYQGEVTVANNGSAALTGWTVQLTLAGGQTVANVWNGINTGTSGTISVRNAAYNGSVGANASTSFGFLVNGSTGTAPGSLTCTSP

SEQ ID NO: 143 - protein, 456 aa, Sphaerisporangium rubeum
MVVIGTAVVVGAGVCAGAGAADAAGTLGAAAAGSGRDFGTAVQAGRLGEAAYVETLDREFTSVTPENEMKWDAVEPARGTFVFTAADRIVEHARARGMKIRGHTLVWHPGLPGWLTNLSSAEFRIAVNNHIATVMGHWRGQIDSWDVVNEAFQDGSGALRSTIFRSRLGDGYIEEAFRAARAADPAAKLCYNDYNIEDADAAKTRAVYAMVRDFKERGVPIDCVGVQSHFNASMPFPANYRRTIEQFAALGVDVQITQLDVDGGAAQAATYRAAVEACVAVPRCTGITVWGVPDHYSWRAPNTPLLFDRDYQKKPAYFAVLEALNAAGGEPTPAPTPTPSPTPTPTPTVTPVPVSCRVTAELWARSRTGYVIKPVTVRNTGRAAVSGWTVTFTLPQGHVVTGSWDAVLTVAGGTVTARNAGHNGTLAPGATATFGFQVRRASGGTALPSGYACRAG

SEQ ID NO: 144 - protein, 557 aa, Phytoactinopolyspora halotolerans
MITRHRRQAILASMALVGATLVVPIGAAQAQDTLRGAADAQGLRMGAAVANGPLSSDAQYRNILGTEFNSVTAENSMKWESLQPSQGQFTFSQGDAIVDFAQANNQAVYGHTLVWHNQTPSWVQNLSGQQLDDAMQTHITTVLDHYEGQVEAWDVANEVIDDGANLRNSFWLQGLGEGYIADAFRYADAADPNAKLYINDYNIDGINAKSNAYYDLVSGLLNQGVPIDGIGLQAHMILGQVPSSLEDNIRRFAQLGLEVRITELDIRMDLPVTQAKLEQQRQDYAAVVDACMSVDGCVGVTTWGFTDAHSWVPDQFGGQGAALPFDENYNKKPAYYGILDTLDGGTPNDTTPPTQPGAPQISDVTSNSASLTWSASSDSGGSGLAGYSVYREQSGNDQLLASPSTNSVTLTGLDPQTQYSVYVVARDGAGNTSSPSTAASFTTQEGPGGGGACDVEYTANNWGGSEGFTASVTITNTGSSQLNGWTLGFTFPGDQSVREGWSATWSQSGADVTAESIGWNDALAPGGSTTVGFNGTYSGSNPEPTEFTLNGEPCSVS

SEQ ID NO: 145 - protein, 466 aa, Glycomyces paridis
MHTRKRRRALIAGVVTVATAALGIGLSTQFASAQTTLRGAADEAGIDIGIAVDANQLQNNATYRNLVATEFNSLTAENAMKWDATEPSDNSWNFSGADAIVDFAEDNDQTVHGHTFVWHSQTPQWVQNLSASAMQAAMVDHINTLANRYEGRVDSWDVVNEVVSDNNGAMRDSFWRNTLGDGYITTAFQTARAADPNADLYINDYSIEGDNAKSDRIYTIAQGLNSQNLIDGVGFQSHLILGQVPSTMEANLQRFIDLGLKVRITELDIRIQTPADANELQQQANDYERVVSICAELADCSGVTVWGLRDADSWVPGVFPGYGAPLLFDDNFGKKPAYNAVLEALGGDGSEEPTTTDPGTTTPPPTGDGDCTAEIDVVNDWGSGWQGNVLITADNGAVNGWTLTWTWPSGQSITSSWNATVTSSGSSVTASDVGWNANIAAGQTMNAWGFVGSGSSAAPQVTCTAD

SEQ ID NO: 146 - protein, 486 aa, Streptomonospora alba
MRLVRHGGHETPRRTRAGSARRVAGAALATFLGAAVLAPAGPAAADSPLRDHAAQQGFEIGAALGVDHLQNDSQFADLAATEFNAATAENSMKWESTEPSRGQFDFSGADAFMDFAQQNNQKVRGHTLVWHSQLPSWVENGNFSQSELRSVMEDHIDEVAGRYAGEVAYWDVANEIFEGDGSWRNSVFYDTLGPDFVADALRMTAEADPNAELWLNDYSIDGINAKSDAYYNLIQDLQAQGVPIDGIGLQAHLINGQVPGDLQQNIQRFADLGIKVAITELDVRIDMPASQSELQQQAQDYRAVMDACLAVNGCVGVTVWGIVDKYSWVPDTFEGEGAPLLFNDDYQPKPAYDAVHEALGGSSDDDDDDDNGGGEDPPPTGPCEVSYSVANEWNSGFTGQVTVTNGGSSALNGWDLQFDFSGGQQITNGWNADWSQSGSTVTASNTEWNGSVPAGGSVDIGFNASHSGSNPEPGSFALNGESCSVS

SEQ ID NO: 147 - DNA, 1413 bp, Synthetic construct
  source 1..1413: mol_type = other DNA
atgacgccaagttcaatgtcccgtcgggcccgcgttgcgtcagctcttgccatcgtgacgttgggcgctactatcgctactacgattcctgcccaagccgccggctctacattacaagcggctgcttctgaaagtggtcgttatttcggtacagcgattgctgcgtttaaattgaatgattcaacgtacagctccattgcgaatcgggagttcaatatgattacagcagaaaatgaaatgaaaatggacgcgactgagccgtctcagaataatttttcgtattcgagtggcgatcagattcttaattgggcacggtctaatggtaagcgtgttcgtggccatgcattggcctggcattcgcagcaaccggggtggatgcaaaatatgtccggtacacagttacgcaatgccatgttgaatcatgtgacgcaggttgctacgcattataaaggcaagatttatgcatgggatgtcgttaatgaagcctatgcggactctggtggggggcgtcgtgacagtaatcttcaacggaccggtgatgattggattgaagctgcttttcgcgctgcgcgggcggcagatccaggtgccaaactttgctataatgattataatacagacaattggacgtgggctaaaacacagggtgtctacaacatggttaaagattttaaagctcgtggggtgccgattgattgcgttggctttcaatctcatttcaattccgggtcgccgtatccgtcaaattatcgtacaaccttacaaaatttcgccgccctgggtgtggaagttcagattaccgaattagatatcgaaggttcggggcaacaacaggcacagacttatgccaatgtcgtcgctgattgcttggcagtgaaggcttgcaccggtatcacagtttggggcgtcagagactcagactcttggcgcagctccgggacgccgttgttgtttgatggcagcggtaataaaaaggctgcgtatacctctaccttggacgctcttaaccggggtggagtcccgactgatccgaccactccgccgacagaccccacgacaccacccactgatcccacgacaccgcctaccgatcctaccactccgcctacggatcctacaaccccacccacggatccaactggccgttgcaccgcgagccttgcgattgccaacgcctggccgggtggatatcaggccacggtcacggtcaaagcagggtccagtagtatcaatggatggcgcgttacattaccttctggcgtttccactagtaacctgtggaatggcgtcctggcgaacggagtggtcacgaatgccccttataatggctctgttggcgcggggcaatccacgacctttggctttgtcggcaatggctcggcgcccagtgctggtagcgtgacttgtgcctga

(19) United States
(12) Patent Application Publication
(10) Pub. No.: US 2025/0059497 A1
(43) Pub. Date: Feb. 20, 2025

(54) PRODUCTION OF ALCOHOL AND ALCOHOL PRECURSORS BY GENETICALLY MODIFIED ETHANOLOGENIC BACTERIA

(71) Applicant: Chemical Evolution Ltd., Calgary (CA)

(72) Inventors: Dustin LILLICO, Calgary (CA); Trevor RANDALL, Calgary (CA); Alejandra ENRIQUEZ, Calgary (CA); Markus WEISSENBERGER, Calgary (CA)

(21) Appl. No.: 18/781,807

(22) Filed: Jul. 23, 2024

(30) Foreign Application Priority Data
Aug. 4, 2023 (CA) 3208555

(51) Int. Cl. C12N 1/20; C12N 9/42; C12N 15/74; C12P 7/06

(52) U.S. Cl. CPC C12N 1/20; C12N 9/2437; C12N 9/2445; C12N 15/74; C12Y 302/01004; C12Y 302/01021; C12P 7/065

(57) ABSTRACT

A genetically modified ethanologenic organism which comprises:
at least one endoglucanase (cen-like) polynucleotide sequence selected from the group consisting of:
   an endoglucanase A-like (cenA-like) polynucleotide sequence which has at least 70% sequence coverage to SEQ 1; and at least 70% sequence identity to SEQ 1;
   an endoglucanase B-like (cenB-like) polynucleotide sequence which has least 70% sequence coverage to SEQ 3; and at least 70% sequence identity to SEQ 3; and
   an endoglucanase C-like (cenC-like) polynucleotide sequence which has at least 70% sequence coverage to SEQ 5; and at least 70% sequence identity to SEQ 5;
a β-glucosidase 1 (bgl1) polynucleotide sequence which has at least 70% sequence coverage to SEQ 9 or SEQ 20; and at least 70% sequence identity to SEQ 9 or SEQ 20; and
an exoglucanase (cex-like) polynucleotide sequence which has least 70% sequence coverage to

[symbol]

### REFERENCE TO AN ELECTRONIC SEQUENCE LISTING

The contents of the electronic sequence listing (20241106_SequenceListing_ST26_23156203US1.xml: Size: 286,790 bytes; and Date of Creation: Nov. 6, 2024) is herein incorporated by reference in its entirety.

### FIELD OF THE INVENTION

The present invention relates to a method to produce alcohols and alcohol precursors through genetically manipulated ethanologenic bacteria that degrades cellulose and ferments the remaining sugar source.

### BACKGROUND OF THE INVENTION

Biofuel is increasingly becoming a necessity in order to wean off the human consumption of fossil fuels in aspects of everyday life, transport and home heating being the largest two industries of focus. As an alternative energy source to oil and coal, the main feedstock for the production of bioethanol and other bioalcohols is starch which can yield its sugar much more readily than cellulose. This is due to the difference in structure as starch contains glucose molecules connected through α-1,4 linkages and cellulose comprises of glucose molecules attached through β-1,4 linkages. The β-1,4 linkages allow for crystallization of the cellulose, leading to a more rigid structure, which is more difficult to break down.

The limitation that comes from solely concentrating on the production of bioethanol by extracting sugars from starches prevents the utilization of the larger portion of biomass which comes in the form of lignocellulosic biomass (contains lignin, cellulose, and hemicellulose) present in almost every plant on earth. A delignification reaction allows the recovery of cellulose from those lignocellulosic plants. Further degradation of the cellulose generates cellobiose and/or glucose which can be utilized for the production of ethanol using various biology-based processes.

Seen as a sustainable alternative to gasoline and with the goal of alleviating many countries' dependence on foreign oil, the bioethanol industry is still hampered by its dependence on corn or sugar cane as main sources of fuel, as they are both rich in starch. It is estimated that about 45% of all corn production in the U.S. is directed to ethanol fuel production. This is a situation which has disastrous consequences when the prices of gasoline decline significantly low making corn-based bioethanol unsustainable on a price viewpoint.

Across the world, many other large ethanol-producing countries, including China and Brazil, have shown some struggles in ethanol production from biomass as many companies are carrying large debts from the implementation of such processes in conjunction with large processing plants having to be shut down or decrease production.

In Asia, palm oil prices have recently increased to their highest levels in years, which, in turn, will hamper the ability of Indonesia and Malaysia to produce local biofuel. Oil palm trunk is a valuable and plentiful resource in those countries to generate biofuels and biochemicals. Oil palm trunk contains a large amount of starch which is more readily solubilized in water, compared to cellulose. The starch can then be heated and hydrolyzed to glucose by amylolytic enzymes without pre-treatment. However, the conventional Oil palm trunk treatment requires high capital and operational costs and is therefore prohibitive to market entry. Moreover, the treatment carries a high probability of microbial contamination during starch processing which significantly reduces purity and poses additional complications in refining procedures.

In Europe, the biofuel industry (both biodiesel and bioethanol production) depends heavily on food-based feedstock like virgin vegetable oils (i.e., rapeseed, palm oil, soy) for biodiesel and corn, wheat, and sugar beet for bioethanol. Simultaneously, concerns have been raised that making fuel out of crops displaces other crops and can inflate food prices. These concerns are leading to policy changes that incentivize a shift away from food-based biofuels.

The pivot from starches to cellulose for the production of glucose is preferable as it will cease to use a food source to generate glucose. However, the costs to do so are currently prohibitive. Cellulosic ethanol as it is called relies on the non-food component of a plant to be used to generate ethanol. This would allow the replacement of the current more widespread approach of making bioethanol by using corn or sugarcane. The diversity and abundance of these types of cellulose-rich plants would allow for food resources to remain largely intact and capitalize on the waste generated from these food resources (such as cornstalk) to generate ethanol. Other cellulose sources such as straws, algae and even trees fall under cellulose-rich biomass sources which can be used in generating ethanol if a commercially viable process is developed.

The reason why starches are preferred to cellulose-rich sources to generate ethanol is that the extraction of glucose from cellulose is substantially more difficult and resource intensive. To better understand the conditions associated with this increased difficulty, it is worthwhile describing the structural similarities and differences between starch and cellulose.

Cellulose and starch are polymers which have the same repeat units of glucose. However, the differences between starch and cellulose can be seen in the way the repeating glucose monomers are connected to one another. In starch, the glucose monomers are oriented in the same direction. In cellulose, each successive glucose monomer is rotated 180 degrees in respect to the previous glucose monomer. This, in turn, ensures that the bonds between each monomeric glucose differs between starch and cellulose. In starch, the bonds (otherwise known as links) are referred to as α-1,4 linkages, in cellulose these bonds are referred to as β-1,4 linkages (see FIG. 1).

The difference between these bonds impacts the chemical characteristics of starch and cellulose. Starch can dissolve in warm water while cellulose does not. Starch can be digested by humans, cellulose cannot. In general, starch is structurally weaker than cellulose partly due to its geometrical make-up which is less crystalline than cellulose. Starch is, at its core, a method for plants to store energy; therefore extracting sugars from starch is much easier than to do so from cellulose as the latter's core function is to provide structural support.

As the main component of lignocellulosic biomass, cellulose is a biopolymer consisting of many glucose units connected through β-1,4-glycosidic bonds (see FIG. 1). Glucose has two isomers: α-glucose (present in starches as branched polymers) and β-glucose (present in cellulose connected via α-1,4-glycosidic bond with one β-glucose monomer rotated by 180 degrees relative to its neighbour). A cellulose molecule can comprise between hundreds to thousands of glucose units. Since the cellulose molecules are linear, due in part to intermolecular hydrogen bonding, neighboring cellulose molecules can be very closely packed and, in turn, provide the structural strength necessary to support plants.

### Hydrolysis of Cellulose

The hydrolysis of cellulose is the rate limiting step in the conversion of cellulose into biofuel. The processes currently using cellulose as a starting material for bioethanol production require the conversion of cellulose into cellobiose, further processing to generate glucose, and the final generation of ethanol. The fermentation of glucose using ethanologenic organisms is what leads to the production of ethanol. While that last step in biofuel production has been mastered for some time, the rate limiting step of cellulose hydrolysis is the most crucial one which hinders a wider acceptance of biofuels. The difficulty in overcoming this conversion of cellulose into glucose lies with the fact that cellulose has a crystalline structure which renders its conversion to glucose quite difficult because of the close packing of multiple cellulose polymers. This close packing imparts cellulose its inherent stability under a variety of chemical conditions. For example, cellulose polymers are generally insoluble in water, as well as a number of organic solvents. Furthermore, cellulose is also generally insoluble when exposed to weak acids or bases.

In general, there are two main approaches to hydrolyze cellulose: chemical and enzymatic. The chemical method resorts to the use of concentrated strong acids to hydrolyze cellulose under conditions of high temperature and pressure. Many different types of acids, such as HCl and H₂SO₄, have been used in the past to achieve this. The use of one of these acids usually results in at least one of the following drawbacks: corrosion of the reaction vessel, difficulty of disposing of the discharged reactants, and others. The biofuel industry is generally reticent to use chemically hydrolyzed cellulose because of the presence of toxic by-products in the resulting glucose. These by-products, if introduced in the fermentation step, will negatively affect the delicate balance of the fermenting yeast.

### Cost of Enzymatic Hydrolysis

It is known that the costs to extract biofuel from cellulose are higher than when doing so from starch. It is estimated that, on average, depending on location and availability of biomass, the cost for cellulose conversion is about 50% more that starch conversion to glucose. This means that there currently is a clear barrier to producers for using cellulose rather than corn or other starch resources to generate glucose from biomass.

It is generally understood that roughly half of the total cost of producing biofuel from cellulose stems from the price of the enzymes (cellulases and hemicellulases). The generation of enzymes for enzymatic hydrolysis of cellulose is a time-consuming process and large volumes of enzyme are required to render the process commercially viable. One possible approach is to improve the rate of the hydrolysis reaction which, in turn, would result in a decrease in the overall cost of the process.

The enzymatic approach to hydrolyzing cellulose uses enzymes to carry out the hydrolysis reaction. Enzymes, such as cellulases (comprising endo-1,4-β-glucanases: exo-1,4-β-glucanases; and β-glucosidases) are used for the conversion of cellulose into glucose, however each requires extensive controls in place to maximize the reaction rates the enzymatic approach is expected to provide. Conditions such as temperature, pH, salinity; concentration of substrate and product are all factors that may affect enzyme activity with even small deviations of these parameters from the enzyme's optimal conditions resulting in loss of function. Overall, with many controls needing to be taken into consideration with the enzymatic hydrolysis of cellulose along with strict enzyme-specific functional conditions, such a method can render the process cost prohibitive in some cases and/or limit their implementation.

The enzymatic hydrolysis of cellulose is, as seen from the above, limited by the structure of cellulose itself but also by the approaches taken to degrade it into a biofuel. The production of a robust, low-cost process from cellulose has not yet been achieved. In this sense, genetically modified organisms have been employed for part of the entirety of the cellulose to ethanol pathway.

U.S. Pat. No. 4,496,656A describes a process for production of cellulase according to the present invention thus comprises culturing a cellulase-producing microorganism belonging to Cellulomonas uda CB4 in a cellulose-containing medium and recovering the cellulase produced from the culture broth. According to the present invention, because the bacteria belonging to Cellulomonas uda CB4 is capable of producing a cellulase having a high activity, not found in the reports of the prior art, in a culture medium, it is possible to produce a cellulase having a high crystalline cellulose decomposing activity comparable to those produced from a mold within a short cultivation period of two days.

In the paper titled “Expression of a cellulase gene in Zymomonas mobilis” by Misawa et al. (J. Biotechnology, 1988, 7 (3), 167-177), it was reported that a cellulase (CMCase) gene of Cellulomonas uda CB4 was introduced into Zymomonas mobilis NRRL B-14023 on pZA22, a cloning vector for Zymomonas, by conjugal transfer. Z. mobilis carrying this gene synthesized cellulase immunologically identical with that of C. uda CB4, by transcriptional read-through from the promotor of the chloramphenicol resistance (chloramphenicol acetyltransferase) gene within pZA22. A strong promotor containing a translation initiation signal was obtained from Z. mobilis NRRL B-14023 chromosomal DNA. By gene fusion between this Zymomonas promotor fragment and the truncated cellulase gene, the activity of cellulase synthesized in Z. mobilis reached 0.78 units per ml culture, six-fold higher than that by transcriptional read-through from the promotor of the chloramphenicol resistance gene.

In the paper titled “Expression of an endoglucanase gene of Pseudomonas fluorescens var. cellulosa in Zymomonas mobilis” by Lejeune et al. (FEMS Microbiology Letters, 1988, 49 (3), 363-366), the authors reported that by using a broad host-range, mobilizable plasmid vector, the endoglucanase gene eglX from Pseudomonas fluorescens var. cellulosa was cloned and expressed in the bacterial ethanologen Zymomonas mobilis. The enzyme was intracellular in this new host. It was produced throughout the growth phase and was not repressed by glucose. We postulate that transcription of the eglX gene was initiated from a promoter of the vector in Z. mobilis.

In the paper titled “Heterologous Expression and Extracellular Secretion of Cellulolytic Enzymes by Zymomonas mobilis” by Linger et al. (Appl Environ Microbiol. 2010 October: 76 (19): 6360-6369), the authors reported that by using a technique known as consolidated bioprocessing (CBP), the initial steps toward achieving a single organism to convert pretreated lignocellulosic biomass to ethanol in the fermentation host Zymomonas mobilis were investigated. This was achieved by expressing heterologous cellulases and subsequently examining the potential to secrete these cellulases extracellularly. Numerous strains of Z. mobilis were found to possess endogenous extracellular activities against carboxymethyl cellulose, suggesting that this microorganism may harbor a favorable environment for the production of additional cellulolytic enzymes. The heterologous expression of two cellulolytic enzymes. E1 and GH12 from Acidothermus cellulolyticus, was examined. Both proteins were successfully expressed as soluble, active enzymes in Z. mobilis although to different levels. While the E1 enzyme was less abundantly expressed, the GH12 enzyme comprised as much as 4.6% of the total cell protein. Additionally: fusing predicted secretion signals native to Z. mobilis to the N termini of E1 and GH12 was found to direct the extracellular secretion of significant levels of active E1 and GH12 enzymes. The subcellular localization of the intracellular pools of cellulases revealed that a significant portion of both the E1 and GH12 secretion constructs resided in the periplasmic space. Our results strongly suggest that Z. mobilis is capable of supporting the expression and secretion of high levels of cellulases relevant to biofuel production, thereby serving as a foundation for developing Z. mobilis into a CBP platform organism.

In the paper titled “Evaluation of Cellulase Production by Zymomonas mobilis” by Todhanakasem and Jittjang (Bioresources, 2017, 12 (1). 1165-1178), the authors report the use of Z. mobilis as a potential microbe for consolidated bioprocessing to convert lignocellulosic biomass to fermentable sugars while at the same time producing ethanol. To achieve this goal. Z. mobilis must be evaluated for the production of cellulolytic enzyme. This work reports on the potential of intracellular and extracellular crude extracts from Z. mobilis ZM4 and TISTR 551 to hydrolyze various cellulosic materials including carboxymethylcellulose (CMC), delignified rice bran, microcrystalline cellulose, and filter paper. Crude intracellular extracts from ZM4 and TISTR 551 showed high endoglucanase activity with CMC substrates at an optimal pH of 6 to 7 and temperature range of 30 to 40° C. The endoglucanase activity from the crude extracts was significantly higher than the exoglucanase activity. Of the high crystalline celluloses substrates tested, the best results were obtained for the hydrolysis of delignified rice bran by crude intracellular enzyme extracts of Z. mobilis TISTR 551.

In the paper by Vasan et al. titled “Cellulosic ethanol production by Zymomonas mobilis harboring an endoglucanase gene from Enterobacter cloacae” (Bioresource Technol. 2011, 102 (3), 2585), the authors report the use of a 2.25-kb fragment conferring cellulase activity from Enterobacter cloacae (isolated from the gut of the wood feeding termite, Heterotermes indicola), cloned in Escherichia coli. The cloned fragment contained a 1083-bp ORF which could encode a protein belonging to glycosyl hydrolase family 8. The cellulase gene was introduced into Zymomonas mobilis strain Microbial Type Culture Collection centre (MTCC) on a plasmid and 0.134 filter paper activity unit (FPU)/ml units of cellulase activity was observed with the recombinant bacterium. Using carboxymethyl cellulose and 4% NaOH pretreated bagasse as substrates, the recombinant strain produced 5.5% and 4% (v/v) ethanol respectively; which was threefold higher than the amount obtained with the original E. cloacae isolate. The recombinant Z. mobilis strain could be improved further by simultaneous expression of cellulase cocktails before utilizing it for industrial level ethanol production.

In light of the state of the art, it is clear that there is an unmet need for a process to generate alcohol and alcohol precursors from a lignocellulosic biomass material that is not reliant on enzymatic cocktails due to their cost. In that respect, the use of a single organism that contains cellulolytic as well as fermentation capabilities is much more highly attractive as it directly targets the still high costs of bioethanol production.

### SUMMARY OF THE INVENTION

Preferred embodiments of the present invention are related to novel organisms for alcohol and alcohol precursor production using cellulose-based materials. More particularly, the invention encompasses methods for the genetic modifications of an ethanologenic organism which allows the organism to produce alcohol and alcohol precursors in association with cellulose degradation through the introduction of genes isolated from prokaryotic and/or eukaryotic organisms. These genes associated with cellulose degradation are then integrated into an ethanologenic organism. Through the inclusion of cellulose degrading genes into an organism that natively does not contain them, this invention extends metabolic pathways beyond those present natively in the ethanologenic organism itself for the purpose of producing ethanol.

According to a first aspect of the present invention, there is provided polynucleotides and their corresponding polypeptide sequences from microorganisms that allows for the expression of cellulose hydrolytic proteins in bacteria for the purpose of producing an alcohol or alcohol precursor. Gene and promoter sequences were introduced into the ethanologenic organism using techniques such as two-step allelic exchange or using expression vectors. Natural promoters and ribosomal binding sites (RBS) from the bacterial expression strain were attached upstream of the gene's start codon to induce gene expression within the bacterium. Methods to transfer these genes from cloning vectors into the organisms of interest are also briefly outlined and are modelled after the protocol seen for the knock in or knock out of genes in a different bacterium, Pseudomonas aeruginosa, using the aforementioned two-step allelic exchange, however these defined methods are not meant to limit the scope of the invention and thus CRISPR and other undefined cloning methods can also be used.

According to a preferred embodiment of the present invention, there is provided a genetically modified ethanologenic organism which comprises an endoglucanase A-like (cenA-like) polynucleotide sequence, wherein said cenA-like polynucleotide sequence has at least 70% sequence coverage to SEQ 1, and at least 70% sequence identity to SEQ 1.

According to a preferred embodiment of the present invention, there is provided a genetically modified ethanologenic organism which comprises an endoglucanase A-like (cenA-like) polynucleotide sequence selected from the group consisting of: SEQ1: SEQ 21: SEQ 22: SEQ 23: SEQ 24: SEQ 25: SEQ 26; and SEQ 27.

According to a preferred embodiment of the present invention, SEQ 1, upon transcription and translation, provides an endoglucanase A-like (CENA-like) polypeptide SEQ 2. According to a preferred embodiment of the present invention, there is provided a genetically modified ethanologenic organism which comprises a CENA-like polypeptide sequence which has at least 70% sequence coverage to SEQ 2, and at least 30% sequence identity to SEQ 2.

According to a preferred embodiment of the present invention, there is provided a genetically modified ethanologenic organism which comprises an endoglucanase A-like (CENA-like) polypeptide sequence selected from the group consisting of: SEQ 2: SEQ 28: SEQ 29: SEQ 30: SEQ 3: SEQ 32: SEQ 33; SEQ 34: SEQ 35: SEQ 36: SEQ 37: SEQ 38: SEQ 39: SEQ 40: SEQ 41: SEQ 42: SEQ 43: SEQ 44; and SEQ 45.

According to a preferred embodiment of the present invention, there is provided a genetically modified ethanologenic organism which comprises an endoglucanase B-like (cenB-like) polynucleotide sequence, wherein said cenB-like polynucleotide sequence has at least 70% sequence coverage to SEQ 3, and at least 70% sequence identity to SEQ 3.

According to a preferred embodiment of the present invention, there is provided a genetically modified ethanologenic organism which comprises an endoglucanase B-like (cenB-like) polynucleotide sequence selected from the group consisting of: SEQ 3: SEQ 46: SEQ 47: SEQ 48: SEQ 49: SEQ 50; and SEQ 51.

According to a preferred embodiment of the present invention, SEQ 3, upon transcription and translation, provides an endoglucanase B-like (CENB-like) polypeptide SEQ 4. According to a preferred embodiment of the present invention, there is provided a genetically modified ethanologenic organism which comprises a CENB-like polypeptide sequence which has at least 70% sequence coverage to SEQ 4, and at least 25% sequence identity to SEQ 4.

According to a preferred embodiment of the present invention, there is provided a genetically modified ethanologenic organism which comprises an endoglucanase B-like (CENB-like) polypeptide sequence selected from the group consisting of: SEQ 4: SEQ 52: SEQ 53: SEQ 54: SEQ 55: SEQ 56: SEQ 57: SEQ 58: SEQ 59: SEQ 60: SEQ 61: SEQ 62: SEQ 63: SEQ 64: SEQ 65: SEQ 64: SEQ 65: SEQ 66; SEQ 67: SEQ 68: SEQ 69: SEQ 70: SEQ 71: SEQ 72: SEQ 73: SEQ 74; and SEQ 75.

According to a preferred embodiment of the present invention, there is provided a genetically modified ethanologenic organism which comprises an endoglucanase C-like (cenC-like) polynucleotide sequence, wherein said cenC-like polynucleotide sequence has at least 70% sequence coverage to SEQ 5, and at least 70% sequence identity to SEQ 5.

According to a preferred embodiment of the present invention, there is provided a genetically modified ethanologenic organism which comprises an endoglucanase C-like (cenC-like) polynucleotide sequence, wherein said cenC-like polynucleotide sequence is selected from the group consisting of: SEQ5; SEQ 76; and SEQ 77.

According to a preferred embodiment of the present invention, SEQ 5, upon transcription and translation, provides an endoglucanase C-like (CENC-like) polypeptide SEQ 6. According to a preferred embodiment of the present invention, there is provided a genetically modified ethanologenic organism which comprises a CENC-like polypeptide sequence which has at least 70% sequence coverage to SEQ 6, and at least 25% sequence identity to SEQ 6.

According to a preferred embodiment of the present invention, there is provided a genetically modified ethanologenic organism which comprises an endoglucanase C-like (CENC-like) polypeptide sequence selected from the group consisting of: SEQ 6: SEQ 78: SEQ 79: SEQ 80: SEQ 81: SEQ 82: SEQ 83: SEQ 84: SEQ 85: SEQ 86: SEQ 87: SEQ 88: SEQ 89: SEQ 90: SEQ 91: SEQ 92; and SEQ 93.

According to a preferred embodiment of the present invention, there is provided a genetically modified ethanologenic organism which comprises a β-glucosidase 1 (bgl1) polynucleotide sequence, wherein said bgl/polynucleotide sequence has at least 70% sequence coverage to SEQ 9 or SEQ 20, and at least 70% sequence identity to SEQ 9 or SEQ 20.

According to a preferred embodiment of the present invention, there is provided a genetically modified ethanologenic organism which comprises a β-glucosidase 1 (bgl1) polynucleotide sequence selected from the group consisting of: SEQ 9: SEQ 20: SEQ 94: SEQ 95: SEQ 96: SEQ 97: SEQ 98: SEQ 99; and SEQ 100.

According to a preferred embodiment of the present invention, SEQ 9 or SEQ 20, upon transcription and translation, provides a β-glucosidase 1 (BGL1) polypeptide SEQ 10. According to a preferred embodiment of the present invention, there is provided a genetically modified ethanologenic organism which comprises a BGL1 polypeptide sequence which has at least 70% sequence coverage to SEQ 10, and at least 35% sequence identity to SEQ 10.

According to a preferred embodiment of the present invention, there is provided a genetically modified ethanologenic organism which comprises a β-glucosidase 1 (BGL1) polypeptide sequence wherein said BGL1 polypeptide sequence is selected from the group consisting of: SEQ 10: SEQ 101: SEQ 102: SEQ 103: SEQ 104: SEQ 105: SEQ 106: SEQ 107: SEQ 108: SEQ 109: SEQ 110: SEQ 111: SEQ 112: SEQ 113: SEQ 114: SEQ 115: SEQ 116: SEQ 117: SEQ 118; and SEQ 119.

According to a preferred embodiment of the present invention, there is provided a genetically modified ethanologenic organism which comprises an exoglucanase (cex-like) polynucleotide sequence, wherein said cex-like polynucleotide sequence has at least 70% sequence coverage to SEQ 7 or SEQ 147, and at least 70% sequence identity of SEQ 7 or SEQ 147.

According to a preferred embodiment of the present invention, there is provided a genetically modified ethanologenic organism which comprises an exoglucanase (cex-like) polynucleotide sequence, wherein said cex-like polynucleotide sequence is selected from the group consisting of: SEQ 7: SEQ 120; SEQ 121: SEQ 122: SEQ 123: SEQ 124: SEQ 125: SEQ 126: SEQ 127; and SEQ 147.

According to a preferred embodiment of the present invention, SEQ 7 or SEQ 147, upon transcription and translation, provides an exoglucanase (CEX-like) polypeptide SEQ 8. According to a preferred embodiment of the present invention, there is provided a genetically modified ethanologenic organism which comprises a CEX-like polypeptide sequence which has at least 70% sequence coverage to SEQ 8, and at least 35% sequence identity to SEQ 8.

According to a preferred embodiment of the present invention, there is provided a genetically modified ethanologenic organism which comprises an exoglucanase (CEX-like) polypeptide sequence wherein said CEX-like polypeptide sequence is selected from the group consisting of: SEQ 8: SEQ 128; SEQ 129: SEQ 130: SEQ 131: SEQ 132: SEQ 133: SEQ 134: SEQ 135: SEQ 136: SEQ 137: SEQ 138; SEQ 139: SEQ 140: SEQ 141: SEQ 142: SEQ 143: SEQ 144: SEQ 145; and SEQ 146.

According to a to a preferred embodiment of the present invention, there is provided a genetically modified ethanologenic organism which comprises:
at least one endoglucanase (cen-like) polynucleotide sequence selected from the list consisting of:
i) an endoglucanase A-like (cenA-like) polynucleotide sequence which has at least 70% sequence coverage to SEQ 1, and at least 70% sequence identity to SEQ 1;
ii) an endoglucanase B-like (cenB-like) polynucleotide sequence which has at least 70% sequence coverage to SEQ 3, and at least 70% sequence identity to SEQ 3; and
iii) an endoglucanase C-like (cenC-like) polynucleotide sequence which has at least 70% sequence coverage to SEQ 5, and at least 70% sequence identity to SEQ 5; and
an exoglucanase (cex-like) polynucleotide sequence with at least 70% sequence coverage to SEQ 7 or SEQ 147, and at least 70% sequence identity to SEQ 7 or SEQ 147.

According to a to a preferred embodiment of the present invention, there is provided a genetically modified ethanologenic organism which comprises:
at least one endoglucanase (cen-like) polynucleotide sequence selected from the list consisting of:
i) an endoglucanase A-like (cenA-like) polynucleotide sequence which has at least 70% sequence coverage to SEQ 1, and at least 70% sequence identity to SEQ 1;
ii) an endoglucanase B-like (cenB-like) polynucleotide sequence which has at least 70% sequence coverage to SEQ 3, and at least 70% sequence identity to SEQ 3; and
iii) an endoglucanase C-like (cenC-like) polynucleotide sequence which has at least 70% sequence coverage to SEQ 5, and at least 70% sequence identity to SEQ 5; and
a β-glucosidase 1 (bgl1) polynucleotide sequence with at least 70% sequence coverage to SEQ 9 or SEQ 20; and at least 70% sequence identity to SEQ 9 or SEQ 20.

According to a to a preferred embodiment of the present invention, there is provided a genetically modified ethanologenic organism which comprises:
an endoglucanase (cen-like) polynucleotide sequence which is comprising of:
   i) endoglucanase A-like (cenA-like) polynucleotide sequence which has at least 70% sequence coverage to SEQ 1; and at least 70% sequence identity to SEQ 1;
   ii) endoglucanase B-like (cenB-like) polynucleotide sequence which has at least 70% sequence coverage to SEQ 3; and at least 70% sequence identity to SEQ 3; and
   iii) endoglucanase C-like (cenC-like) polynucleotide sequence which has at least 70% sequence coverage to SEQ 5; and at least 70% sequence identity to SEQ 5; and
a β-glucosidase 1 (bgl1) polynucleotide sequence which has at least 70% sequence coverage to SEQ 9 or SEQ 20; and at least 70% sequence identity to SEQ 9 or SEQ 20; and
an exoglucanase (cex-like) polynucleotide sequence which has at least 70% sequence coverage to SEQ 7 or SEQ 147; and at least 70% sequence identity to SEQ 7 or SEQ 147.

According to a preferred method of the present invention, the ethanologenic organism is selected from the genus of Aspergillus, Mucor, Zymomonas. Escherichia, Clostridia, Bacillus, and Pseudomonas, however this list by no means is meant to limit the scope of the invention. Preferably, the ethanologenic organism is the bacteria Zymomonas mobilis.

According to another aspect of the present invention, ethanologenic bacterial cells were genetically modified by inserting a bacterial promoter sequence into the bacterial genome, immediately followed by a cellulose hydrolytic gene(s) related to the degradation of cellulose. In some embodiments, the native promoter and polynucleotide sequence could also include the promoter and ribosomal binding site (RBS) including various combinations from the following genes: Glyceraldehyde-3-phosphate dehydrogenase, type I (gap) (P_{gap}), Thiamine pyrophosphate protein TPP binding domain-containing protein (pdc) (P_{pdc}), the EF-Tu transcription factor (P_{tuf}), Phosphopyruvate hydratase (eno) (Pₑₙₒ), 2-Dehydro-3-deoxyphosphogluconate aldolase/4-hydroxy-2-oxoglutarate aldolase (eda) (P_{eda}), glucose-6-phosphate dehydrogenase (zwf) (P_{zwf}), ROK family protein (frk) (P_{frk}), Carboxymethylenebutenolidase (clcD1) (P_{cleD1}), as well as Glucosamine-fructose-6-phosphate aminotransferase (glmS) (Pgims), however this list of possible promoters is in no way meant to limit the scope of the invention. In preferred embodiments, the promoter and RBS polynucleotide sequence to regulate cellulose degradation for ethanol production is the native promoter for the Glyceraldehyde-3-phosphate dehydrogenase, type I (gap) gene (P_{gap}), obtained from Z. mobilis.

According to a preferred embodiment of the present invention, the utilized promoter sequence is followed by the polynucleotide sequence for genes related to the production of enzymes associated with cellulose degradation, such as endoglucanase A-like (cenA-like), endoglucanase B-like (cenB-like), endoglucanase C-like (cenC-like), exoglucanase (cex-like), or β-glucosidase 1 (bgl1) and any combination thereof. In a preferred embodiment of the invention, at least one of each type of enzyme (an endoglucanase, an exoglucanase, and a β-glucosidase) are all used in concert to fully degrade cellulose to glucose.

According to a preferred embodiment of the present invention, cenA-like genes can be acquired from, but by no means limited to, the following bacterial species: Cellulomonas uda, Cellulomonas palmilytica, Cellulomonas gelida, Cellulomonas xiejunii, Neisseria sicca, Sanguibacter suaedae, Isoptericola dokdonensis, Sanguibacter antarcticus, Cryptosporangium aurantiacum, Isoptericola halotolerans, Nocardiopsis lucentensis, and Pseudoduganella lutea. These listed organisms are exemplary only and are in no way meant to limit what organisms these genes can be acquired from, nor to limit the scope of the invention.

According to a preferred embodiment of the present invention, cenB-like genes can be acquired from, but by no means limited to, the following bacterial species: Cellulomonas uda, Cellulomonas gelida, Micromonospora luteifusca, Micromonospora orduensis, Asanoa ishikariensis, Catelliglobosispora koreensis, Roseateles terrae, Thermobifida halotolerans, Catellatospora tritici, Micromonospora noduli, Anaerolineae bacterium, Reinekea marinisedimentorum, and Evansella caseinilytica. These listed organisms are exemplary only and are in no way meant to limit what organisms these genes can be acquired from, nor to limit the scope of the invention.

According to a preferred embodiment of the present invention, cenC-like genes can be acquired from, but by no means is limited to, the following bacterial species: Cellulomonas uda, Cellulomonas palmilytica, Cellulomonas fulva, Zhihengliuella halotolerans, Saccharothrix ecbatanensis, Cellulomonas terrae, Ruania rhizosphaerae, Alteromonadaceae bacterium Bs31, and Catellatospora tritici. These listed organisms are exemplary only and are in no way meant to limit what organisms these genes can be acquired from, nor to limit the scope of the invention.

According to a preferred embodiment of the present invention, cex-like genes can be acquired from, but by no means is limited to, the following bacterial species: Cellulomonas uda, Cellulomonas palmilytica, Cellulomonas cellasea, Saccharothrix syringae, Promicromonospora iranensis, Glycomyces paridis, Micromonospora fulviviridis, Couchioplanes caeruleus, Streptomonospora alba, Phytoactinopolyspora halotolerans, Cellulomonas wangsupingiae, Xylanimonas cellulosilytica, Actinoplanes lutulentus, or Micromonospora saelicesensis. These listed organisms are exemplary only and are in no way meant to limit what organisms these genes can be acquired from, nor to limit the scope of the invention.

According to a preferred embodiment of the present invention, bgl1 genes can be acquired from, but by no means is limited to, the following eukaryotic species: Aspergillus niger, Aspergillus luchuensis, Penicillium vulpinum, Sanghuangporus baumii, Penicillium rolfsii, Trichoderma gamsii, Halenospora varia, Daldinia childiae, Fusarium solani, Glaciozyma antarctica, Monascus purpureus, and Trichophyton interdigitale. These listed organisms are exemplary only and are in no way meant to limit what organisms these genes can be acquired from, nor to limit the scope of the invention.

According to a preferred embodiment of the present invention, the cenA-like cenB-like, cenC-like, cex-like, and bgl1 genes are obtained from the organism Cellulomonas uda (cenA-like B-like C-like cex-like) and Aspergillus niger (bgl1), respectively. In other embodiments, a native promoter sequence can be used to bolster the production of proteins and/or molecules utilized in the production of an alcohol or alcohol precursor through the hydrolysis of cellulose.

According to another embodiment of the present invention, the ethanologenic bacterial cells have been genetically modified to have improved degradation of cellulose through the addition of both prokaryotic and eukaryotic gene(s) which when transcribed and translated generate cellulose hydrolytic enzymes. In some embodiments, the transcription of these genes is driven by a promoter sequence, and can include, but is not limited to, any combination of the P_{gap}-cenA-like, P_{gap}-cenB-like, P_{gap}-cenC-like, P_{gap}-cex-like or P_{gap}-bgl1 gene cassettes which have been integrated into a bacterial cell.

### BRIEF DESCRIPTION OF THE ACCOMPANYING FIGURE

The invention may be more completely understood in consideration of the following description of various embodiments of the invention in connection with the accompanying FIGURE, in which:

FIG. 1 is a schematic representation of the structure of cellulose.

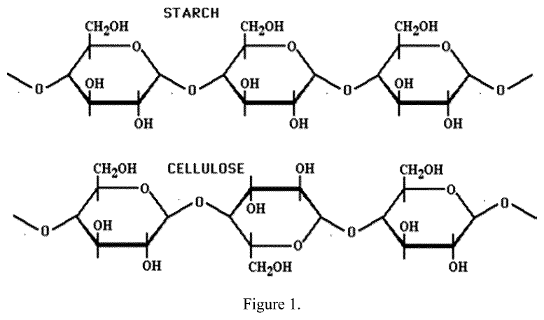

### DETAILED DESCRIPTION OF THE INVENTION

It is known to the person skilled in the art that some species of ethanologenic bacteria contain and express endogenous endoglucanase or exogenous endoglucanase enzymes with functional cellulase activities. According to a preferred embodiment of the present invention, there is provided a bacteria with enhanced innate capabilities to increase the efficiency of cellulose degradation by said bacteria through the incorporation of exogenous endoglucanase enzymes. Furthermore, a preferred embodiment of the present invention expands the array of cellulase-containing enzymes of said bacteria through the addition of genes which allow further cellulose degradation activities.

According to a preferred embodiment of the present invention, there are provided genetic modifications to ethanologenic organisms which allow for the degradation of cellulose to glucose with the purpose of producing an alcohol or alcohol precursor from an inexpensive cellulosic feedstock using bacterial species modified with a cenA-like polynucleotide (SEQ 1) or, upon transcription and translation of said cenA-like polynucleotide, a CENA-like polypeptide (SEQ 2).

According to a preferred embodiment of the present invention, there are provided genetic modifications to ethanologenic organisms which allow for the degradation of cellulose to glucose with the purpose of producing an alcohol or alcohol precursor from an inexpensive cellulosic feedstock using bacterial species modified with a cenB-like polynucleotide (SEQ 3) or, upon transcription and translation of said cenB-like polynucleotide, a CENB-like polypeptide (SEQ 4).

According to a preferred embodiment of the present invention, there are provided genetic modifications to ethanologenic organisms which allow for the degradation of cellulose to glucose with the purpose of producing an alcohol or alcohol precursor from an inexpensive cellulosic feedstock using bacterial species modified with a cenC-like polynucleotide (SEQ 5) or, upon transcription and translation of said cenC-like polynucleotide, a CENC-like polypeptide (SEQ 6).

According to a preferred embodiment of the present invention, there are provided genetic modifications to ethanologenic organisms which allow for the degradation of cellulose to glucose with the purpose of producing an alcohol or alcohol precursor from an inexpensive cellulosic feedstock using a bacterial species modified with a cex-like polynucleotide (SEQ 7 or SEQ 147) or, upon transcription and translation of said cex-like polynucleotide, a CEX-like polypeptide (SEQ 8).

According to a preferred embodiment of the present invention, there are provided genetic modifications to ethanologenic organisms which allow for the degradation of cellulose to glucose with the purpose of producing an alcohol or alcohol precursor from an inexpensive cellulosic feedstock using a bacterial species modified with a bgl1 polynucleotide (SEQ 9 or SEQ 20) or, upon transcription and translation of said bgl1 polynucleotide, a BGL1 polypeptide (SEQ 10).

According to a preferred embodiment of the present invention, there are provided genetic modifications to ethanologenic organisms which include any combination of cenA-like (SEQ 1), cenB-like (SEQ 3), cenC-like (SEQ 5), cex-like (SEQ 7 or SEQ 147) and bgl1 (SEQ 9 or SEQ 20) allowing for increased degradation of cellulose to glucose with the intent to produce an alcohol or alcohol precursor from inexpensive feedstock as a source of cellulose.

According to a preferred embodiment of the present invention, there are provided genetic modifications to ethanologenic organisms that contain a combination of polynucleotide sequences including the exoglucanase cex-like polynucleotide (SEQ 7 or SEQ 147), the β-glucosidase bgl1 polynucleotide (SEQ 9 or SEQ 20), and at least one of the endoglucanase genes cenA-like (SEQ 1), cenB-like (SEQ 3) or cenC-like (SEQ 5) polynucleotides.

### Definitions

Within the context of this invention all terms and technical parameters described fall within their commonly known meanings as known by individuals within the region of science that the proposed invention is associated with unless otherwise stated. Furthermore, unless otherwise indicated, all techniques utilized within this invention are commonly conducted within the fields of molecular biology, cell biology, biochemistry, and microbiology.

An alcohol or alcohol precursor is any molecule that is obtained as an intermediate, by-product, or co-product of the conversion of a cellulosic material to an alcohol. It is known to those skilled in the art that said alcohol precursor can be an oligosaccharide, di-saccharide, monosaccharide, organic acid and their corresponding salts, aldehyde, ketone, etc.

Biofuels within the context of this invention is defined as any fuel for which is produced using biological material.

Bioethanol within the context of this invention is defined as ethanol that is produced from lignocellulosic biomass.

Cellulosic ethanol is defined within the context of this invention as ethanol that is produced through the degradation of cellulose.

Cellobiose is defined within the context of this invention as the dimer of glucose formed from the degradation of cellulose.

Cellulose hydrolysis within the context of this invention is defined as the degradation of cellulose via hydrolytic processes.

Hydrolysis within the context of this invention is defined as a chemical or microbiological reaction which facilities the breaking of chemical bonds between molecules.

A monomer is defined within this invention as a single molecule that can, under specific conditions, be combined with other molecules, including itself, to generate more complex structures called polymers.

A polymer is defined within the context of this invention as a material comprised of repeating subunits of the same or different molecule.

A biopolymer is defined within the context of this invention as a polymer that is typically produced by the cells of living organisms. By way of example, cellulose is considered a biopolymer.

A polynucleotide within the context of this invention is defined as the collection of individual nucleotides in any organization or size.

A polypeptide within the context of this invention is defined as the combination of multiple peptides of any organization or size that relates to the amino acid sequence. The term polypeptide and protein within the context of this invention can be used interchangeably.

A vector within the context of this invention refers to the composition of a polynucleotide within the intended purpose of introducing nucleic acids into one or more organism types. Vectors are further defined based on their functional purpose and can be designated as expression vectors, cloning vectors, plasmids, or shuttle vectors.

The term expression within the context of this invention refers to the generation of a polypeptide sequence which is produced based on its polynucleotide sequence or gene.

The term promoter in the context of this invention is used to describe the nucleic acid sequence for the regulation and binding of polymerases for the purpose of the transcribing of a gene. This promoter can be native to an organism, or a non-endogenous promoter can be introduced into an organism to alter the regulation of gene expression.

The term gene refers to a sequence of DNA that encodes for a specific polypeptide sequence. A gene can include both sequences between coding regions (introns) and the encoding sequence itself (exon).

The term recombinant in the context of this invention refers to the modification or alteration of a sequence associated with either a polypeptide or polynucleotide sequence. Recombination can be utilized for altering expression and coding segments of a gene of interest that would produce a non-native or non-naturally occurring product.

The term homology or homologous refers to the level of similarity between two or more polypeptide or polynucleotide sequences.

The terms transfection, transformation, or introduced refer to the addition of polynucleotide sequence(s) that would normally be considered exogenous to organism. This may include the addition of a polynucleotide directly to the genome of an organism or the transfer of a plasmid and/or vector.

Within the context of this patent the terms native or natural refers to polypeptide and/or polynucleotides present within the organism prior to any modification. These native or naturally occurring polypeptides and/or polynucleotides would be present or produced by the organism without any external alterations.

The term metabolic pathway refers to the subsequential biochemical reactions involved in the formation of a biologically relevant product within an organism.

Within the context of this invention the terms knock-in and knock-out refer to the addition or removal of DNA sequences within an organism and can also be interchangeable with the terms insertion and deletion, respectively.

The term promoter within the context of this invention refers to a sequence of DNA that is responsible for the initiation of DNA transcription and thus polypeptide formation.

A coding sequence within the context of this invention refers to a sequence of polynucleotides or DNA that facilitates the generation of a protein through transcription and translational processes.

Genetic modification or related statements herein refer to the alteration of the genetic code of an organism which includes the insertion or deletion of DNA sequences within an organism. Within the context of this invention, genetic modification could include insertion and maintenance of an expression vector into the organism, or the direct modification of the organism's genome by directly adding or deleting genes through a process like two-step allelic exchange or CRISPR.

The term ribosomal binding site (RBS) refers to the region within a polynucleotide sequence that allows for the appropriate binding of the ribosome to facilitate the translation of a polynucleotide sequence to produce the corresponding polypeptide sequence, which includes the terms protein, enzyme, and plasmid.

The term cloning vector herein refers to a polynucleotide sequence or plasmid that can be replicated within a host organism for storage or amplification purposes. A cloning vector may contain all the necessary regulatory sequences needed to facilitate the transcription and translation of a protein.

The term unmodified promoter is defined as a promoter sequence by which has been unaltered or exists within the host organism itself.

The term two-step allelic exchange is referred to the process by which a gene of interest is either interested or deleted from an organism through specific selective conditions. The insertion or deletion of a specific gene of interest is done so through the utilization of distinct polynucleotide sequences which allow for the exchange of genetic material between two sources.

The term CRISPR cloning is defined as a process by which the gene of interest is inserted or removed from an organism's genome using the CRISPR-CAS9 cloning system.

The terms upstream and downstream refer to regions of polynucleotides which are found prior to or after a specific gene of interest within a plasmid and/or genome of an organism.

The term enzyme within this invention defines a polypeptide sequence, specifically in the form of a protein, that can modify a biological molecule or take part within its generation through direct or indirect interactions. The process by which an enzyme influences the modification and/or production of a biological molecule and/or product is termed enzymatic activity.

The term open reading frame (ORF) refers to the collection of nucleotides which are found in between the start and stop codons of a polypeptide encoding DNA sequence.

The term codon(s) refers to 3 adjacent nucleotides in a polynucleotide sequence that are used by the cell to “decode” the polynucleotide sequence when the polynucleotide sequence is translated to make the polypeptide sequence and are responsible defining the order of protein residues in a polypeptide sequence based on this code. Based on a 3-letter code, and 4 different nucleotide bases, these codons include 64 different combinations that are able to be used by the cell, which with some redundancy codes for 22 possible protein residues, as well as 1 start and 3 stop codons.

A start and stop codon refer to a nucleotide codon sequence comprised of three specific nucleotides in succession of each other which allows for the identification of the initiation (start) and termination (stop) for the transcription of a polypeptide sequence.

The term encoded refers to the polypeptide sequence that is obtained from a polynucleotide sequence after the polynucleotide sequence had been transcribed and translated.

The combined term transcribed and translated (or the combined process of transcription and translation) refers to the process by which a polynucleotide sequence is used to produce a polypeptide sequence.

Metabolic engineering herein refers to the alteration of an organism's metabolic pathway potential. This can include both the deactivation and/or altering of pre-existing metabolic pathways of an organism or the inclusion of additional metabolic processes.

Sequence alignment herein refers to a bioinformatic technique by which two polynucleotide sequences or two polypeptide sequences are arranged or aligned in such a way as to identify regions of similarity between a reference sequence (the sequence that is known) and the quarry sequence (the sequence to be compared to the reference sequence). Those skilled in the art know that alignment algorithms such as, but by no means limited to, the BLAST, ALIGN, or CLUSTAL algorithms can be used to obtain this information for polynucleotide or polypeptide sequences, respectively.

Percentage sequence identity or percentage identity herein refers to the similarity between 2 sequences that have been processed through a sequence alignment, to provide insight into how similar aligned sequences are at either the nucleotide or peptide level for polynucleotide or polypeptide sequences, respectively. The percentage identity is used to determine the similarity of a query sequence to a reference sequence.

Percentage sequence coverage or percentage coverage refers to the number of aligned nucleotides or peptides in a query sequence relative to the length of the reference sequence. The percentage coverage provides an indication of how much of the reference polynucleotide or polypeptide sequence is covered by the query sequence, allowing for instance the lengths of the found genes or proteins to be compared.

The BLASTN algorithm was used herein as one method to determine the percentage identity and percentage coverage between one or even multiple different polynucleotide sequences with respect to an inputted reference sequence, allowing for the determination of the percentage identity and percentage coverage of one or many query sequences to said reference sequence. One of ordinary skill in the art will recognize that search results from a BLASTN search will be influenced by the search parameters used in the search. Therefore, for all BLASTN searches done with respect to this invention to identify other sequences which have been catalogued in the NCBI polynucleotide databases relative to a reference include the following parameters:
Search set parameters are comprising of “standard databases (nr ect)”, with the specific database used being the “Nucleotide collection (nr/nt)”, and no exclusions or limitations were placed on the search (all default parameters)
Program selection algorithm parameters include the highly similar sequences (known as the megablast algorithm) (the default parameter)
Algorithm parameters altered include the Max target sequences, which was set at 5000, otherwise all default parameters are used for relevant searches (other parameters in “General parameters”, and all parameters in “Scoring parameters” and “Filters and masking” are default)

The BLASTP algorithm was used herein as one method to determine the percentage identity and percentage coverage between one or even multiple different polypeptide sequences with respect to an inputted reference sequence, allowing for the determination of the percentage identity and percentage coverage of one or many query sequences to said reference sequence. One of ordinary skill in the art will recognize that search results from a BLASTP search will be influenced by the search parameters used in the search. Therefore, for all BLASTP searches done with respect to this invention to identify other sequences which have been catalogued in the NCBI polypeptide databases relative to a reference include the following parameters:
Search set parameters are comprising of “standard databases (nr ect)”, with the specific database used being the “Non-redundant protein sequences (nr)”, and no exclusions or limitations were placed on the search (all default parameters)
Program selection algorithm parameters include the BLASTP (known as the protein-protein BLAST algorithm) (the default parameter)
Algorithm parameters altered include the Max target sequences, which was set at 5000, otherwise all default parameters are used for relevant searches (other parameters in “General parameters”, and all parameters in “Scoring parameters” and “Filters and masking” are default). Notable default parameters include an “Expect Threshold and word size of 0.05 and 5, respectively in the general parameters, the usage of the BLOSUM62 matrix with gap costs of Existence: 11 and Extension: 1 for the Scoring parameters, and no filter or masking components selected.

The phrases “substantially similar” or “substantially identical” in the context of at least 2 nucleic acid sequences or at least 2 polypeptide sequences typically means that a polynucleotide, polypeptide, or region or domain of a polypeptide has a percentage coverage of at least 70%, 75%, 80%, 85%, 90%, 95%, 97%, 99%, or even 99.5%, and at least 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% percentage identity to the reference sequence. Some polynucleotide or polypeptide sequences that fall in this category are sequences that share genetic or protein homology to the reference sequence.

The terms genetic and protein homology, or homologous sequences refer to polynucleotide sequences or translated polypeptide sequences that have a similar or identical function in the cell. For example, 2 different proteins share a similar or identical function even though they were isolated from 2 different organisms. Polynucleotide sequences with homology are generally understood to have similar or identical biochemical functionality.

The term endoglucanase refers to a protein with a certain enzymatic activity to degrade cellulose by attacking random internal 1,4-β-linkages between the glucose units in cellulose.

The term exoglucanase refers to a protein with a certain enzymatic activity to degrade cellulose by releasing cellobiose or glucose from the nonreducing end of the cellulose polymer.

The term glucosidase refers to a protein with enzymatic activity to degrade cellobiose to glucose.

The term ethanologenic organism refers within this invention to an organism that can produce ethanol as part of its native metabolic processes.

### Enzymes and Promoters for Cellulosic Ethanol Production

Example polypeptide sequences for enzymes involved in cellulose degradation that can be integrated into ethanologenic organisms are provided in the sequence listing. The expression and production of these sequences within the cell are partially driven by the genetic polynucleotide promoter and ribosomal binding site sequences as provided in the sequence listings: SEQ 11 (P_{gap}), SEQ 12 (P_{pdc}), SEQ 13 (P_{tuf}), SEQ 14 (Pₑₙₒ), SEQ 15 (P_{eda}), SEQ 16 (P_{zwf}), SEQ 17 (P_{frk}), SEQ 18 (P_{clcD1}), and SEQ 19 (P_{glmS}). The invention is not limited to the use of these polynucleotide sequences and their respective polypeptide sequences. Those of ordinary skill in the art know that organisms of a wide variety of species commonly express and utilize homologous proteins, which contain insertions, substitutions and deletions in the polypeptide sequences of the polynucleotide sequences listed above, and effectively provide a similar function. For example, the protein sequences for CENA-like from Cellulomonas uda or Isoptericola dokdonensis or Isoptericola halotolerans; or for CENB-like from Cellulomonas uda, Asanoa ishikariensis, or Catellatospora tritici; or for CENC-like from Cellulomonas uda or Saccharothrix ecbatanensis or Ruania rhizosphaerae; or for CEX-like from Cellulomonas uda or Couchioplanes caeruleus or Promicromonospora iranensis; or for BGL1 from Aspergillus niger or Penicillium vulpinum or Halenospora varia may differ to different degrees from the polypeptide sequences seen between these organisms yet maintain similar or identical functions of the protein within the organism with respect to enzymatic function. Protein sequences comprising such variations are included within the scope of the present invention and are considered substantially or sufficiently similar to the reference polynucleotide sequences provided above and upon transcription and translation their respective polypeptide sequences. Although it is not intended that the present invention is limited by any theory by which it achieves its advantageous result, it is believed and supported by biochemical knowledge that the identity between polypeptide sequences that is necessary to maintain proper functionality is related to maintaining the tertiary structure (3D) of the polypeptide. This maintenance of the tertiary structure is associated with the specific interactive/catalytic portions of the protein sequence and will therefore have the desired activity, and it is contemplated that a protein including these interactive sequences in the proper special context will have this activity.

A person of ordinary skill in the art knows that many different amino acids contain properties between each other and can serve similar functions in the final polypeptide sequence. Thus, when one amino acid is changed with another amino acid from this group, such as a non-polar amino acid, an uncharged polar amino acid, a charged polar acidic amino acid, or a charged polar basic amino acid, some polypeptide functionality is generally maintained. For example, it is known that the uncharged polar amino acid serine may be substituted for the uncharged polar amino acid threonine in a polypeptide without substantially altering the protein structure and functionality. Whether a given substitution will affect the functionality of the enzyme may be determined without undue experimentation using synthetic techniques and screening assays known to one of ordinary skill in the art.

A person of ordinary skill in the art will recognize that changes in the protein sequence, resulting from individual single or multi-nucleotide substitutions, deletions, or additions to a polynucleotide will lead to changes in the resulting translated polypeptide sequence. Small mutations, such as the change of an amino acid from one to another, or the addition or elimination of single amino acids, or a small to moderate percentage of amino acids from the encoded polypeptide sequence can be considered “sufficiently similar” when the alteration results in the substitutions of an amino acid with a chemically similar amino acid. Thus, any number of amino acid residues in a polypeptide chain, selected from a group of integers from 1-50, can be so altered. Thus, for example, 1, 2, 3, 5, 10, 12, 20, 32, 41, or even 50 alterations can be made. Conservatively modified variants typically provide similar biological activity to the unmodified variants and typically provide similar biological activity as the unmodified polypeptide sequence from which they are derived. For example, modification of CENA-like, CENB-like, CENC-like, CEX-like, and BGL1 to yield functional proteins generally have a sequence identity of at least 40%, 50%, 60%, 70%, 80%, or 90%, preferably a sequence identity of greater than 50%, of the native protein to allow processing of its native substrate. Tables of conserved substitution provide lists of functionally similar amino acids. Amino acids in polypeptide chains that are similar to one another include the following groups: (1) Serine(S), Threonine (T): (2) Aspartic acid (D), Glutamic acid (E): (3) Asparagine (N), Glutamine (Q): (4) Alanine (A), Leucine (L), and Isoleucine (I).

### Suitable Polynucleotide and Polypeptide Sequences for CENA-like, CENB-like, CENC-like, CEX-like, and BGL1

A person of ordinary skills in the art will recognize that many different organisms will have functionally similar polynucleotide and polypeptide sequences (or homology between the sequences), however there may be differences between these sequences when compared to a reference sequence. As examples, suitable polynucleotides and their corresponding polypeptide sequences for cellulose degradation can be seen below. Note that the following sequences by no means are meant to limit the scope of the invention. In fact, any substantially similar polynucleotide sequences or substantially similar produced polypeptide sequences for the CENA-like, CENB-like, CENC-like, CEX-like, and BGL1 genes and corresponding proteins with similar function or similarity to these genes/proteins in the cellulose degradation pathway can also be used for producing alcohol and alcohol precursors including.

According to a preferred embodiment of the present invention, the endoglucanase A-like (cenA-like) polynucleotide sequence SEQ 1, is one endoglucanase utilized for producing alcohol and alcohol precursors including cellulosic ethanol. In this embodiment, cenA-like is under the transcriptional control of a native promoter and a ribosomal binding site. However, in other embodiments of the invention, cenA-like polynucleotide sequences that are homologous and/or substantially similar to SEQ 1 may also be used in the present invention to produce alcohol and alcohol precursors including cellulosic ethanol. Polynucleotide sequences for endoglucanase A-like in these embodiments have at least 70% sequence coverage, or more preferably greater than 80%, 90%, 95%, or most preferentially greater than 99% sequence coverage to SEQ 1, and sequence identities of at least 70%, or more preferentially greater than 80%, 90%, 95%, 97% sequence identity, and most preferentially greater than 99% sequence identity to SEQ 1. These polynucleotide sequences may include, but by no means limited to, the following sequences: SEQ 1: SEQ 21: SEQ 22: SEQ 23: SEQ 24: SEQ 25: SEQ 26; and SEQ 27.

According to a preferred embodiment of the present invention, the endoglucanase A-like (CENA-like) polypeptide SEQ 2 is utilized for producing alcohol and alcohol precursors including cellulosic ethanol by the cell, whereby SEQ 2 is produced from the transcription and translation of the endoglucanase A-like (cenA-like) polynucleotide SEQ 1. However, in other embodiments of the invention, polypeptide sequences that are homologous and substantially similar to SEQ 2 may also be used in the present invention to produce alcohol and alcohol precursors including cellulosic ethanol. CENA-like polypeptide sequences in these embodiments have at least 70% sequence coverage to SEQ 2, or more preferentially greater than 75%, 80%, 85%, 90%, 95%. 97%, or most preferentially greater than 99% sequence coverage to SEQ 2, and a sequence identity of at least 30% to SEQ 2, or more preferably greater than 35%, 40%, 45%, 50%, 55%, 60%, 65%, 70%, 75%, 80%, 85%, 90%, 95%. 98% sequence identity, or most preferentially greater than 99% sequence identity to SEQ 2. These polypeptide sequences may include, but are by no means limited to, the following sequences: SEQ 2: SEQ 28: SEQ 29: SEQ 30: SEQ 31: SEQ 32: SEQ 33: SEQ 34: SEQ 35: SEQ 36: SEQ 37: SEQ 38: SEQ 39: SEQ 40: SEQ 41: SEQ 42: SEQ 43: SEQ 44; and SEQ 45.

According to a preferred embodiment of the present invention, the endoglucanase B-like (cenB-like) polynucleotide sequence SEQ 3, is one endoglucanase utilized for producing alcohol and alcohol precursors including cellulosic ethanol production. In this embodiment, cenB-like is under the transcriptional control of a native promoter and a ribosomal binding site. However, in other embodiments of the invention, polynucleotide sequences that are homologous and/or substantially similar to SEQ 3 may also be used in the present invention to produce alcohol and alcohol precursors including cellulosic ethanol. The cenB-like polynucleotide sequences for endoglucanase B-like in these embodiments have at least 70% sequence coverage, or more preferably greater than 80%, 90%, or most preferentially greater than 99% sequence coverage to SEQ 3, and sequence identities of at least 70%, or more preferentially greater than 80%, 85%, 90%, 95% sequence identity, and most preferentially greater than 99% sequence identity to SEQ 3. These polynucleotide sequences may include, but by no means are limited to, the following sequences: SEQ 3: SEQ 46: SEQ 47: SEQ 48: SEQ 49; SEQ 50; and SEQ 51.

According to a preferred embodiment of the present invention, the endoglucanase B-like (CENB-like) polypeptide SEQ 4 is utilized to produce producing alcohol and alcohol precursors including cellulosic ethanol by the cell, whereby SEQ 4 is produced from the transcription and translation of the endoglucanase B-like (cenB-like) polynucleotide SEQ 3. However, in other embodiments of the invention, polypeptide sequences that are homologous and substantially similar to SEQ 4 may also be used in the present invention to produce alcohol and alcohol precursors including cellulosic ethanol. CENB-like polypeptide sequences in these embodiments have at least 70% sequence coverage to SEQ 4, or more preferentially greater than 75%, 80%, 85%, 90%, 95%. 97%, 98%, or most preferentially greater than 99% sequence coverage of SEQ 4, and a sequence identity of at least 25% to SEQ 4, or more preferably greater than 30%, 35%, 40%, 45%, 50%, 55%, 60%, 65%, 70%, 75%, 80%, 85%, 90%, 95% sequence identity, or most preferentially greater than 99% sequence identity to SEQ 4. These polypeptide sequences may include, but are by no means limited to, the following sequences: SEQ 4: SEQ 52: SEQ 53: SEQ 54: SEQ 55: SEQ 56: SEQ 57: SEQ 58: SEQ 59: SEQ 60; SEQ 61: SEQ 62: SEQ 63: SEQ 64: SEQ 65: SEQ 66: SEQ 67: SEQ 68: SEQ 69; SEQ 70: SEQ 71: SEQ 72: SEQ 73: SEQ 74; and SEQ 75.

According to a preferred embodiment of the present invention, the endoglucanase C-like (cenC-like) polynucleotide sequence SEQ 5 is one endoglucanase utilized for producing alcohol and alcohol precursors including cellulosic ethanol production. In this embodiment, cenC-like is under the transcriptional control of a native promoter and a ribosomal binding site. However, in other embodiments of the invention, polynucleotide sequences that are homologous and/or substantially similar to SEQ 5 may also be used in the present invention to produce alcohol and alcohol precursors including cellulosic ethanol. The cenC′-like polynucleotide sequences for endoglucanase C-like in these embodiments have at least 70% sequence coverage, or more preferably greater than 90%, or most preferentially greater than 99% sequence coverage to SEQ 5, and sequence identities of at least 70%, or more preferentially greater than 80%, 85%, 90% sequence identity, and most preferentially greater than 99% sequence identity to SEQ 5. The cenC′-like polynucleotide sequences may include, but by no means are limited to, the following sequences: SEQ5; SEQ 76; and SEQ 77.

According to a preferred embodiment of the present invention, the endoglucanase C-like (CENC-like) polypeptide SEQ 6 is utilized for producing alcohol and alcohol precursors including cellulosic ethanol by the cell, whereby SEQ 6 is produced from the transcription and translation of the endoglucanase C-like (cenC-like) polynucleotide SEQ 5. However, in other embodiments of the invention. CENC-like polypeptide sequences that are homologous and substantially similar to SEQ 6 may also be used in the present invention to produce alcohol and alcohol precursors including cellulosic ethanol. CENC-like polypeptide sequences in these embodiments have at least 70% sequence coverage to SEQ 6, or more preferentially greater than 75%, 80%, 85%, 90%, 95%. 97%, 98%, or most preferentially greater than 99% sequence coverage to SEQ 6, and a sequence identity of at least 25% to SEQ 6, or more preferably greater than 30%, 35%, 40%, 45%, 50%, 60%, 70%, 75%, 80%, 90%, 95%, or 98% sequence identity, or most preferentially greater than 99% sequence identity to SEQ 6. These polypeptide sequences may include, but are by no means limited to, the following sequences: SEQ 6: SEQ 78: SEQ 79: SEQ 80: SEQ 81: SEQ 82; SEQ 83: SEQ 84: SEQ 85: SEQ 86: SEQ 87: SEQ 88: SEQ 89: SEQ 90: SEQ 91: SEQ 92; and SEQ 93.

According to a preferred embodiment of the present invention, the β-glucosidase 1 (bgl1) polynucleotide sequence SEQ 9 or SEQ 20, is one β-glucosidase utilized for producing alcohol and alcohol precursors including cellulosic ethanol. In this embodiment, bgl1 is under the transcriptional control of a native promoter and a ribosomal binding site. However, in other embodiments of the invention, bgl1 polynucleotide sequences that are homologous and/or substantially similar to SEQ 9 or SEQ 20 may also be used in the present invention to produce alcohol and alcohol precursors including cellulosic ethanol. The bgl1 polynucleotide sequences for β-glucosidase 1 in these preferred embodiments have at least 70% sequence coverage, or more preferably greater than 75%. 85%, 90%, 95%. 97% or most preferentially greater than 99% sequence coverage to SEQ 9 or SEQ 20, and sequence identities of at least 70%, or more preferentially greater than 75%, 80%, 90%, or 95% sequence identity, and most preferentially greater than 99% sequence identity to SEQ 9 or SEQ 20. These polynucleotide sequences may include, but by no means are limited to, the following sequences: SEQ 9: SEQ 20: SEQ 94: SEQ 95: SEQ 96: SEQ 97: SEQ 98: SEQ 99; and SEQ 100.

According to a preferred embodiment of the present invention, the β-glucosidase 1 (BGL1) polypeptide SEQ 10 is utilized to produce alcohol and alcohol precursors including cellulosic ethanol by the cell, whereby SEQ 10 is produced from the transcription and translation of the β-glucosidase 1 (bgl1) polynucleotide SEQ 9 or SEQ 20. However, in other embodiments of the invention, BGL1 polypeptide sequences that are homologous and substantially similar to SEQ 10 may also be used in the present invention to produce alcohol and alcohol precursors including cellulosic ethanol. BGL1 polypeptide sequences in these preferred embodiments will have at least 70% sequence coverage to SEQ 10, or more preferentially greater than 75%, 80%, 85%, 90%, 95%. 97%, 98%, or most preferentially greater than 99% sequence coverage to SEQ 10, and a sequence identity of at least 35% to SEQ 10, or more preferably greater than 40%, 45%, 50%, 60%, 70%, 75%, 80%, 85%, 90%, 95%, or 97% sequence identity, or most preferentially greater than 99% sequence identity to SEQ 10. These polypeptide sequences may include, but are by no means limited to, the following sequences; SEQ 10; SEQ 101; SEQ 102; SEQ 103; SEQ 104; SEQ 105; SEQ 106; SEQ 107; SEQ 108; SEQ 109; SEQ 110; SEQ 111; SEQ 112; SEQ 113; SEQ 114; SEQ 115; SEQ 116; SEQ 117; SEQ 118; and SEQ 119.

According to a preferred embodiment of the present invention, the exoglucanase (cex-like) polynucleotide sequence SEQ 7 or SEQ 147, is one exoglucanase utilized for producing alcohols and alcohol precursors including cellulosic ethanol. In this embodiment, cex-like is under the transcriptional control of a native promoter and a ribosomal binding site. However, in other embodiments of the invention, cex-like polynucleotide sequences that are homologous and/or substantially similar to SEQ 7 or SEQ 147 may also be used in the present invention to produce alcohol and alcohol precursors including cellulosic ethanol. The cex-like polynucleotide sequences for exoglucanase in these preferred embodiments have at least 70% sequence coverage, or more preferably greater than 75%, 80%, 85%, 90%, 95%, 98% or most preferentially greater than 99% sequence coverage to SEQ 7 or SEQ 147, and sequence identities of at least 70%, or more preferentially greater than 75%, 80%, 90%, or 95% sequence identity, and most preferentially greater than 99% sequence identity of SEQ to 7 or SEQ 147. These polynucleotide sequences may include, but by no means are limited to, the following sequences; SEQ 7; SEQ 120; SEQ 121; SEQ 122; SEQ 123; SEQ 124; SEQ 125; SEQ 126; SEQ 127; and SEQ 147.

According to a preferred embodiment of the present invention, the exoglucanase (CEX-like) polypeptide SEQ 8 is utilized to produce producing alcohols and alcohol precursors including cellulosic ethanol by the cell, whereby SEQ 8 is produced from the transcription and translation of the exoglucanase (cex-like) polynucleotide SEQ 7 or SEQ 147. However, in other embodiments of the present invention, CEX-like polypeptide sequences that are homologous and substantially similar to SEQ 8 may also be used in the present invention to produce alcohol and alcohol precursors including cellulosic ethanol. The CEX-like polypeptide sequences for exoglucanase in these preferred embodiments have at least 70% sequence coverage to SEQ 8, or more preferentially greater than 75%, 80%, 85%, 90%, 95%, 97%, 98%, or most preferentially greater than 99% sequence coverage of SEQ 8, and a sequence identity of at least 35% to SEQ 8, or more preferably greater than 40%, 45%, 50%, 60%, 70%, 75%, 80%, 90%, or 95% sequence identity, or most preferentially greater than 99% sequence identity to SEQ 8. These polypeptide sequences may include, but are by no means limited to, the following sequences: SEQ 8; SEQ 128; SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; and SEQ 146.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A genetically modified ethanologenic organism which comprises at least one endoglucanase (cen-like) polynucleotide sequence selected from the group consisting of:
an endoglucanase A-like (cenA-like) polynucleotide sequence with at least 70% sequence coverage to SEQ 1, and at least 70% sequence identity to SEQ 1;
an endoglucanase B-like (cenB-like) polynucleotide sequence with at least 70% sequence coverage to SEQ 3, and at least 70% sequence identity to SEQ 3; and
an endoglucanase C-like (cenC-like) polynucleotide sequence with at least 70% sequence coverage to SEQ 5, and at least 70% sequence identity to SEQ 5.

2. The genetically modified ethanologenic organism according to claim 1, wherein:
said endoglucanase A-like (cenA-like) polynucleotide sequence is selected from the group consisting of: SEQ1; SEQ 21; SEQ 22; SEQ 23; SEQ 24; SEQ 25; SEQ 26; and SEQ 27;
said endoglucanase B-like (cenB-like) polynucleotide sequence is selected from the group consisting of: SEQ 3; SEQ 46; SEQ 47; SEQ 48; SEQ 49; SEQ 50; and SEQ 51; and
said endoglucanase C-like (cenC-like) polynucleotide sequence is selected from the group consisting of: SEQ 5; SEQ 76; and SEQ 77.

3. The genetically modified ethanologenic organism according to claim 1, which, upon transcription and translation under the control of a native or synthetic promoter and Ribosomal binding site (RBS), comprises:
an endoglucanase (CEN-like) polypeptide sequence from the group consisting of:
i. an endoglucanase A-like (CENA-like) polypeptide sequence which has at least 70% sequence coverage to SEQ 2, and at least 30% sequence identity to SEQ 2;
ii. an endoglucanase B-like (CENB-like) polypeptide sequence which has at least 70% sequence coverage to SEQ 4, and at least 25% sequence identity to SEQ 4; and
iii. an endoglucanase C-like (CENC-like) polypeptide sequence which has at least 70% sequence coverage to SEQ 6, and at least 25% sequence identity to SEQ 6.

4. The modified ethanologenic organism according to claim 3 wherein:
said CENA-like polypeptide sequence is selected from the group consisting of: SEQ 2; SEQ 28; SEQ 29; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; SEQ 44; and SEQ 45;
said CENB-like polypeptide sequence is selected from the group consisting of: SEQ 4; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; SEQ 67; SEQ 68; SEQ 69; SEQ 70; SEQ 71; SEQ 72; SEQ 73; SEQ 74; and SEQ 75; and
said CENC-like polypeptide sequence is selected from the group consisting of: SEQ 6; SEQ 78; SEQ 79; SEQ 80; SEQ 81; SEQ 82; SEQ 83; SEQ 84; SEQ 85; SEQ 86; SEQ 87; SEQ 88; SEQ 89; SEQ 90; SEQ 91; SEQ 92; and SEQ 93.

5. A genetically modified ethanologenic organism which comprises:
at least one endoglucanase (cen-like) polynucleotide sequence selected from the group consisting of:
i. an endoglucanase A-like (cenA-like) polynucleotide sequence which has at least 70% sequence coverage to SEQ 1, and at least 70% sequence identity to SEQ 1;
ii. an endoglucanase B-like (cenB-like) polynucleotide sequence which has at least 70% sequence coverage to SEQ 3, and at least 70% sequence identity to SEQ 3; and
iii. an endoglucanase C-like (cenC-like) polynucleotide sequence which has at least 70% sequence coverage to SEQ 5, and at least 70% sequence identity to SEQ 5; and
an exoglucanase (cex-like) polynucleotide sequence which has at least 70% sequence coverage to SEQ 7 or SEQ 147, and at least 70% sequence identity to SEQ 7 or SEQ 147.

6. The genetically modified ethanologenic organism according to claim 5 wherein:
i. said endoglucanase A-like (cenA-like) polynucleotide sequence is selected from the group consisting of: SEQ 1; SEQ 21; SEQ 22; SEQ 23; SEQ 24; SEQ 25; SEQ 26; and SEQ 27;
ii. said endoglucanase B-like (cenB-like) polynucleotide sequence is selected from the group consisting of: SEQ 3; SEQ 46; SEQ 47; SEQ 48; SEQ 49; SEQ 50; and SEQ 51; and
iii. said endoglucanase C-like (cenC-like) polynucleotide sequence is selected from the group consisting of: SEQ 5; SEQ 76; and SEQ 77; and
said exoglucanase (cex-like) polynucleotide sequence is selected from the group consisting of: SEQ 7; SEQ 120; SEQ 121; SEQ 122; SEQ 123; SEQ 124; SEQ 125; SEQ 126; SEQ 127; and SEQ 147.

7. The genetically modified ethanologenic organism according to claim 5, which, upon transcription and translation under the control of a native or synthetic promoter and Ribosomal binding site (RBS), comprises:
at least one an endoglucanase (CEN-like) polypeptide sequence selected from the group consisting of:
i. an endoglucanase A-like (CENA-like) polypeptide sequence which has at least 70% sequence coverage to SEQ 2, and at least 30% sequence identity to SEQ 2;
ii. an endoglucanase B-like (CENB-like) polypeptide sequence which has at least 70% sequence coverage to SEQ 4, and at least 25% sequence identity to SEQ 4; and
iii. an endoglucanase C-like (CENC-like) polypeptide sequence which has at least 70% sequence coverage to SEQ 6, and at least 25% sequence identity to SEQ 6; and
at least one exoglucanase (CEX-like) polypeptide sequence which has at least 70% sequence coverage to SEQ 8; and at least 35% sequence identity to SEQ 8.

8. The genetically modified ethanologenic organism according to claim 7, wherein:
said endoglucanase A-like (CENA-like) polypeptide sequence is selected from the group consisting of: SEQ 2; SEQ 28; SEQ 29; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; SEQ 44; and SEQ 45;
said endoglucanase B-like (CENB-like) polypeptide sequence is selected from the group consisting of: SEQ 4; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; SEQ 67; SEQ 68; SEQ 69; SEQ 70; SEQ 71; SEQ 72; SEQ 73; SEQ 74; and SEQ 75;
said endoglucanase C-like (CENC-like) polypeptide sequence is selected from the group consisting of: SEQ 6; SEQ 78; SEQ 79; SEQ 80; SEQ 81; SEQ 82; SEQ 83; SEQ 84; SEQ 85; SEQ 86; SEQ 87; SEQ 88; SEQ 89; SEQ 90 SEQ 91; SEQ 92; and SEQ 93; and
said exoglucanase (CEX-like) polypeptide sequence is selected from the group consisting of: SEQ 8; SEQ 128; SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; and SEQ 146.

9. A genetically modified ethanologenic organism which comprises:
at least one endoglucanase (cen-like) polynucleotide sequence selected from the group consisting of:
i. an endoglucanase A-like (cenA-like) polynucleotide sequence which has at least 70% sequence coverage to SEQ 1; and at least 70% sequence identity to SEQ 1;
ii. an endoglucanase B-like (cenB-like) polynucleotide sequence which has at least 70% sequence coverage to SEQ 3; and at least 70% sequence identity to SEQ 3; and
iii. an endoglucanase C-like (cenC-like) polynucleotide sequence which has at least 70% sequence coverage to SEQ 5; and at least 70% sequence identity to SEQ 5; and
a β-glucosidase 1 (bgl1) polynucleotide sequence which has at least 70% sequence coverage to SEQ 9 or SEQ 20, and at least 70% sequence identity to SEQ 9 or SEQ 20.

10. The genetically modified ethanologenic organism according to claim 9, wherein:
said endoglucanase A-like (cenA-like) is selected from the group consisting of: SEQ 1; SEQ 21; SEQ 22; SEQ 23; SEQ 24; SEQ 25; SEQ 26; and SEQ 27;
said endoglucanase B-like (cenB-like) is selected from the group consisting of: SEQ 3; SEQ 46; SEQ 47; SEQ 48; SEQ 49; SEQ 50; and SEQ 51;
said endoglucanase C-like (cenC-like) is selected from the group consisting of: SEQ 5; SEQ 76; and SEQ 77; and
said β-glucosidase 1 (bgl1) polynucleotide sequence is selected from the group consisting of: SEQ 9; SEQ 20; SEQ 94; SEQ 95; SEQ 96; SEQ 97; SEQ 98; SEQ 99; and SEQ 100.

11. The genetically modified ethanologenic organism according to claim 9, which, upon transcription and translation under the control of a native or synthetic promoter and Ribosomal binding site (RBS), comprises:
at least one endoglucanase (CEN-like) polypeptide sequence selected from the group consisting of:
i. an endoglucanase A-like (CENA-like) polypeptide sequence which has at least 70% sequence coverage to SEQ 2; and at least 30% sequence identity to SEQ 2;
ii. an endoglucanase B-like (CENB-like) polypeptide sequence which has at least 70% sequence coverage to SEQ 4; and at least 25% sequence identity to SEQ 4; and
iii. an endoglucanase C-like (CENC-like) polypeptide sequence which has at least 70% sequence coverage to SEQ 6; and at least 25% sequence identity to SEQ 6; and
a β-glucosidase 1 (BGL1) polypeptide sequence which has at least 70% sequence coverage to SEQ 10, and at least 35% sequence identity to SEQ 10.

12. The genetically modified ethanologenic organism according to claim 11, wherein:
said endoglucanase A-like (CENA-like) polypeptide sequence is selected from the group consisting of: SEQ 2; SEQ 28; SEQ 29; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; SEQ 44; and SEQ 45;
said endoglucanase B-like (CENB-like) polypeptide sequence is selected from the group consisting of: SEQ 4; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; SEQ 67; SEQ 68; SEQ 69; SEQ 70; SEQ 71; SEQ 72; SEQ 73; SEQ 74; and SEQ 75;
said endoglucanase C-like (CENC-like) polypeptide sequence is selected from the group consisting of: SEQ 6; SEQ 78; SEQ 79; SEQ 80; SEQ 81; SEQ 82; SEQ 83; SEQ 84; SEQ 85; SEQ 86; SEQ 87; SEQ 88; SEQ 89; SEQ 90 SEQ 91; SEQ 92; and SEQ 93; and
said β-glucosidase 1 (BGL1) polypeptide sequence selected from the group consisting of: SEQ 10; SEQ 101; SEQ 102; SEQ 103; SEQ 104; SEQ 105; SEQ 106; SEQ 107; SEQ 108; SEQ 109; SEQ 110; SEQ 111; SEQ 112; SEQ 113; SEQ 114; SEQ 115 SEQ 116; SEQ 117; SEQ 118; and SEQ 119.

13. A genetically modified ethanologenic organism which comprises:
at least one endoglucanase (cen-like) polynucleotide sequence selected from the group consisting of:
an endoglucanase A-like (cenA-like) polynucleotide sequence which has at least 70% sequence coverage to SEQ 1; and at least 70% sequence identity to SEQ 1;
an endoglucanase B-like (cenB-like) polynucleotide sequence which has least 70% sequence coverage to SEQ 3; and at least 70% sequence identity to SEQ 3; and
an endoglucanase C-like (cenC-like) polynucleotide sequence which has at least 70% sequence coverage to SEQ 5; and at least 70% sequence identity to SEQ 5;
a β-glucosidase 1 (bgl1) polynucleotide sequence which has at least 70% sequence coverage to SEQ 9 or SEQ 20; and at least 70% sequence identity to SEQ 9 or SEQ 20; and
an exoglucanase (cex-like) polynucleotide sequence which has least 70% sequence coverage to SEQ 7 or SEQ 147; and at least 70% sequence identity to SEQ 7 or SEQ 147.

14. The genetically modified ethanologenic organism according to claim 13, wherein:
said endoglucanase A-like (cenA-like) polynucleotide sequence is selected from the group consisting of: SEQ 1; SEQ 21; SEQ 22; SEQ 23; SEQ 24; SEQ 25; SEQ 26; and SEQ 27;
said endoglucanase B-like (cenB-like) is selected from the group consisting of: SEQ 3; SEQ 46; SEQ 47; SEQ 48; SEQ 49; SEQ 50; and SEQ 51;
said endoglucanase C-like (cenC-like) polynucleotide sequence is selected from the group consisting of: SEQ 5; SEQ 76; and SEQ 77;
said β-glucosidase 1 (bgl1) polynucleotide sequence selected from the group consisting of: SEQ 9; SEQ 20; SEQ 94; SEQ 95; SEQ 96; SEQ 97; SEQ 98; SEQ 99; and SEQ 100; and
said exoglucanase (cex-like) polynucleotide sequence selected from the group consisting of: SEQ 7; SEQ 120; SEQ 121; SEQ 122; SEQ 123; SEQ 124; SEQ 125; SEQ 126; SEQ 127; and SEQ 147.

15. The genetically modified ethanologenic organism according to claim 13, which upon transcription and translation under the control of a native or synthetic promoter and Ribosomal binding site (RBS), comprises:
at least one endoglucanase (CEN-like) polypeptide sequence selected from the group consisting of:
i. an endoglucanase A-like (CENA-like) polypeptide sequence which has at least 70% sequence coverage to SEQ 2; and at least 30% sequence identity to SEQ 2;
ii. an endoglucanase B-like (CENB-like) polypeptide sequence which has at least 70% sequence coverage to SEQ 4; and at least 25% sequence identity to SEQ 4; and
iii. an endoglucanase C-like (CENC-like) polypeptide sequence which has at least 70% sequence coverage to SEQ 6; and at least 25% sequence identity to SEQ 6;
a β-glucosidase 1 (BGL1) polypeptide sequence which has at least 70% sequence coverage to SEQ 10; and at least 35% sequence identity to SEQ 10; and
an exoglucanase (CEX-like) polypeptide sequence which has at least 70% sequence coverage to SEQ 8; and at least 35% sequence identity to SEQ 8.

16. The genetically modified ethanologenic organism according to claim 15, wherein:
said endoglucanase A-like (CENA-like) polypeptide sequence is selected from the group consisting of: SEQ 2; SEQ 28; SEQ 29; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; SEQ 44; and SEQ 45;
said endoglucanase B-like (CENB-like) polypeptide sequence is selected from the group consisting of: SEQ 4; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; SEQ 67; SEQ 68; SEQ 69; SEQ 70; SEQ 71; SEQ 72; SEQ 73; SEQ 74; and SEQ 75;
said endoglucanase C-like (CENC-like) polypeptide sequence is selected from the group consisting of: SEQ 6; SEQ 78; SEQ 79; SEQ 80; SEQ 81; SEQ 82; SEQ 83; SEQ 84; SEQ 85; SEQ 86; SEQ 87; SEQ 88; SEQ 89; SEQ 90 SEQ 91; SEQ 92; and SEQ 93;
said β-glucosidase 1 (BGL1) polypeptide sequence is selected from the group consisting of: SEQ 10; SEQ 101; SEQ 102; SEQ 103; SEQ 104; SEQ 105; SEQ 106; SEQ 107; SEQ 108; SEQ 109; SEQ 110; SEQ 111; SEQ 112; SEQ 113; SEQ 114; SEQ 115 SEQ 116; SEQ 117; SEQ 118; and SEQ 119; and
said exoglucanase (CEX-like) polypeptide sequence is selected from the group consisting of: SEQ 8; SEQ 128; SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; and SEQ 146.

17. The ethanologenic organism according to claim 13 further comprising a promoter and RBS sequence to drive polynucleotide expression; wherein the genetic material for the promoter/RBS sequences is selected from the group consisting of: SEQ 11; SEQ 12; SEQ 13; SEQ 14; SEQ 15; SEQ 16; SEQ 17; SEQ 18; and SEQ 19.

18. The ethanologenic organism according to claim 13 where the organism is a prokaryotic organism.

19. The ethanologenic organism according to claim 13; where the organism is a bacterium.

20. The ethanologenic organism according to claim 13 where the organism is selected from the group consisting of the genera Aspergillus; Mucor; Zymomonas; Escherichia; Clostridia; Bacillus; and Pseudomonas.

21. The ethanologenic organism according to claim 13 where the organism belongs to the bacterial genus Zymomonas.
